(19) United States
(12) Patent Application Publication
(10) Pub. No.: US 2012/0052130 A1
(43) Pub. Date: Mar. 1, 2012

(54) GPR 119 MODULATORS

(75) Inventors: Etzer Darout, New Haven, CT (US); Michael Paul DeNinno, San Diego, CA (US); Kentaro Futatsugi, Niantic, CT (US); Cristiano Ruch Werneck Guimaraes, Clinton, CT (US); Bruce Allen Lefker, Gales Ferry, CT (US); Vincent Mascitti, Groton, CT (US); Kim Francis McClure, Mystic, CT (US); Michael John Munchhof, Salem, CT (US); Ralph Pelton Robinson, JR., Gales Ferry, CT (US)

(73) Assignee: Pfizer Inc.

(21) Appl. No.: 13/318,152

(22) PCT Filed: Apr. 12, 2010
(86) PCT No.: PCT/IB10/51567

§ 371 Date: Oct. 31, 2011

Related U.S. Application Data
(60) Provisional application No. 61/176,528, filed on May 8, 2009.
(60) Provisional application No. 61/320,462, filed on Apr. 2, 2010.

(51) Int. Cl. A61K 35/56; A61K 31/506; A61K 31/497; A61K 31/55; A61K 31/536; A61K 31/566; A61K 38/22; A61K 31/4985; A61K 31/4965; A61K 38/16; A61K 31/715; A61K 38/26; A61K 31/513; A61K 31/706; A61K 31/517; A61P 3/10; A61P 3/04; A61P 3/08; A61P 3/06; A61P 9/10; A61P 9/00; A61P 19/02; A61P 19/10; A61P 13/12; A61P 7/02; A61P 25/28; A61P 25/18; A61P 25/00; A61P 1/00; C07D 239/47

(52) U.S. Cl. 424/520; 544/319; 514/269; 514/254.07; 514/217.01; 514/230.5; 514/171; 514/5.3; 514/250; 514/252.1; 514/217.11; 514/6.9; 514/54; 514/7.2; 514/249; 514/274; 514/23; 514/266.2

(57) ABSTRACT

Compounds of Formula (I) that modulate the activity of the G-protein-coupled receptor GPFM 19 and their uses in the treatment of diseases linked to the modulation of the G-protein-coupled receptor GPR119 in animals are described herein.

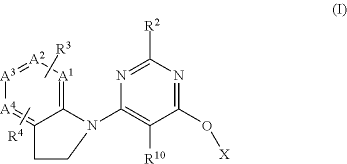

### FIELD OF THE INVENTION

The present invention relates to a new class of fused pyrrolidines, pharmaceutical compositions containing these compounds, and their use to modulate the activity of the G-protein-coupled receptor, GPR119.

### BACKGROUND

Diabetes mellitus are disorders in which high levels of blood glucose occur as a consequence of abnormal glucose homeostasis. The most common forms of diabetes mellitus are Type I (also referred to as insulin-dependent diabetes mellitus) and Type II diabetes (also referred to as non-insulin-dependent diabetes mellitus). Type II diabetes, accounting for roughly 90% of all diabetic cases, is a serious progressive disease that results in microvascular complications (including retinopathy, neuropathy and nephropathy) as well as macrovascular complications (including accelerated atherosclerosis, coronary heart disease and stroke).

Currently, there is no cure for diabetes. Standard treatments for the disease are limited, and focus on controlling blood glucose levels to minimize or delay complications. Current treatments target either insulin resistance (metformin, thiazolidinediones, or insulin release from beta cells (sulphonylureas, exanatide). Sulphonylureas and other compounds that act via depolarization of the beta cell promote hypoglycemia as they stimulate insulin secretion independent of circulating glucose concentrations. One approved drug, exanatide, stimulates insulin secretion only in the presence of high glucose, but must be injected due to a lack of oral bioavailablity. Sitagliptin, a dipeptidyl peptidase IV inhibitor, is a new drug that increases blood levels of incretin hormones, which can increase insulin secretion, reduce glucagon secretion and have other less well characterized effects. However, sitagliptin and other dipeptidyl peptidases IV inhibitors may also influence the tissue levels of other hormones and peptides, and the long-term consequences of this broader effect have not been fully investigated.

In Type II diabetes, muscle, fat and liver cells fail to respond normally to insulin. This condition (insulin resistance) may be due to reduced numbers of cellular insulin receptors, disruption of cellular signaling pathways, or both. At first, the beta cells compensate for insulin resistance by increasing insulin output. Eventually, however, the beta cells become unable to produce sufficient insulin to maintain normal glucose levels (euglycemia), indicating progression to Type II diabetes.

In Type II diabetes, fasting hyperglycemia occurs due to insulin resistance combined with beta cell dysfunction. There are two aspects of beta cell defect dysfunction: 1) increased basal insulin release (occurring at low, non-stimulatory glucose concentrations). This is observed in obese, insulin-resistant pre-diabetic stages as well as in Type II diabetes, and 2) in response to a hyperglycemic challenge, a failure to increase insulin release above the already elevated basal level. This does not occur in pre-diabetic stages and may signal the transition from normo-glycemic insulin-resistant states to frank Type II diabetes. Current therapies to treat the latter aspect include inhibitors of the beta-cell ATP-sensitive potassium channel to trigger the release of endogenous insulin stores, and administration of exogenous insulin. Neither achieves accurate normalization of blood glucose levels and both carry the risk of eliciting hypoglycemia.

Thus, there has been great interest in the discovery of agents that function in a glucose-dependent manner. Physiological signaling pathways which function in this way are well known, including gut peptides GLP-1 and GIP. These hormones signal via cognate G-protein coupled receptors to stimulate production of cAMP in pancreatic beta-cells. Increased cAMP apparently does not result in stimulation of insulin release during the fasting or pre-prandial state. However, a number of biochemical targets of cAMP, including the ATP-sensitive potassium channel, voltage-sensitive potassium channels and the exocytotic machinery, are modulated such that insulin secretion due to postprandial glucose stimulation is significantly enhanced. Therefore, agonist modulators of novel, similarly functioning, beta-cell GPCRs, including GPR119, would also stimulate the release of endogenous insulin and promote normalization of glucose levels in Type II diabetes patients. It has also been shown that increased cAMP, for example as a result of GLP-1 stimulation, promotes beta-cell proliferation, inhibits beta-cell death and thus improves islet mass. This positive effect on beta-cell mass should be beneficial in Type II diabetes where insufficient insulin is produced.

It is well known that metabolic diseases have negative effects on other physiological systems and there is often co-occurrence of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which occur secondary to diabetes such as kidney disease, and peripheral neuropathy. Thus, treatment of the diabetic condition should be of benefit to such interconnected disease states.

### SUMMARY OF THE INVENTION

In accordance with the present invention, a new class of GPR 119 modulators has been discovered. These compounds may be represented by Formula (I), as shown below:

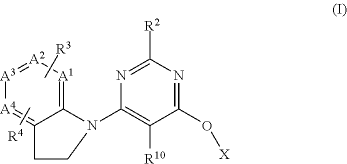

in which

X is

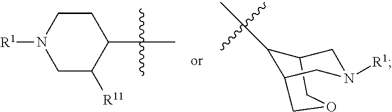

R¹ is —C(O)—O—R⁵ or

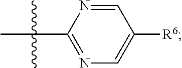

R² is hydrogen, cyano, or methyl;
R³ is hydrogen, OH, halogen, cyano, CF₃, OCF₃, C₁-C₅ alkoxy, or C₁-C₅ alkyl;
R⁴ is SO₂—R⁷ or —NH—(CH₂)₂—OH;
R⁵ is C₁-C₅ alkyl, C₃-C₆ cycloalkyl, or C₃-C₆ cycloalkyl in which one carbon atom of said cycloalkyl moiety is optionally substituted with methyl or ethyl;
R⁶ is CF₃, C₁-C₅ alkyl, halogen, cyano, or C₃-C₆ cycloalkyl;
R⁷ is C₃-C₆ cycloalkyl, C₁-C₅ alkyl, NH₂, or —(CH₂)₂—OH;
R⁸ is hydrogen or C₁-C₅ alkyl,
R⁹ is hydrogen, C₁-C₅ alkyl, C₃-C₆ cycloalkyl, —CH₂—CH₂—OH, —CH₂—CH₂—O—CH₃, —CH₂—CH₂—CH₂—O—CH₃, —CH₂—CH₂—CH₂—OH, 3-oxetanyl, or 3-hydroxycyclobutyl,
R¹⁰ is hydrogen, cyano, nitro, CF₃, OCF₃, C₃-C₆ cycloalkyl, C₁-C₅ alkoxy, or C₁-C₅ alkyl;
R¹¹ is hydrogen, C₁-C₅ alkyl, or halogen; and
A¹, A², A³, and A⁴, are each independently CH, N-oxide, or N;
with the proviso that:
a) no more than 2 of A¹, A², A³, and A⁴ are N;
b) no more than 1 of A¹, A², A³, and A⁴ are N-oxide; and
c) when A¹-A⁴ forms a phenyl ring, X is

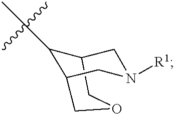

or a pharmaceutically acceptable salt thereof.

The compounds of Formula I modulate the activity of the G-protein-coupled receptor. More specifically the compounds modulate GPR119. As such, said compounds are useful for the treatment of diseases, such as diabetes, in which the activity of GPR119 contributes to the pathology or symptoms of the disease. Examples of such conditions include hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance. The compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

A further embodiment of the invention is directed to pharmaceutical compositions containing a compound of Formula I. Such formulations will typically contain a compound of Formula I in admixture with at least one pharmaceutically acceptable excipient. Such formulations may also contain at least one additional pharmaceutical agent (described herein). Examples of such agents include anti-obesity agents and/or anti-diabetic agents (described herein below). Additional aspects of the invention relate to the use of the compounds of Formula I in the preparation of medicaments for the treatment of diabetes and related conditions as described herein.

### DETAILED DESCRIPTION OF THE INVENTION

The invention may be understood even more readily by reference to the following detailed description of exemplary embodiments of the invention and the examples included therein.

It is to be understood that this invention is not limited to specific synthetic methods of making that may of course vary. It is also to be understood that the terminology used herein is for the purpose of describing particular embodiments only and is not intended to be limiting. The plural and singular should be treated as interchangeable, other than the indication of number:

The headings within this document are only being utilized to expedite its review by the reader. They should not be construed as limiting the invention or claims in any manner.

### DEFINITIONS AND EXEMPLIFICATION

a. “halogen” refers to a chlorine, fluorine, iodine, or bromine atom.
b. “C₁-C₅ alkyl” refers to a branched or straight chained alkyl group containing from 1 to 5 carbon atoms, such as methyl, ethyl, n-propyl, isopropyl, n-butyl, isobutyl, pentyl, etc.
c. “C₁-C₅ alkoxy” refers to a straight or branched chain alkoxy group containing from 1 to 5 carbon atoms, such as methoxy, ethoxy, n-propoxy, isopropoxy, n-butoxy, isobutoxy, pentoxy, etc.
d. “C₃-C₆ cycloalkyl” refers to a nonaromatic ring that is fully hydrogenated and exists as a single ring. Examples of such carbocyclic rings include cyclopropyl, cyclobutyl, cyclopentyl, and cyclohexyl,
e. “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.
f. “patient” refers to warm blooded animals such as, for example, guinea pigs, mice, rats, gerbils, cats, rabbits, dogs, monkeys, chimpanzees, and humans.
g. “treat” refers to the ability of the compounds to either relieve, alleviate, or slow the progression of the patient's disease (or condition) or any tissue damage associated with the disease.
h. “the terms “modulated”, “modulating”, or “modulate(s)”, as used herein, unless otherwise indicated, refers to the activation of the G-protein-coupled receptor GPR119 with compounds of the present invention.
i. “pharmaceutically acceptable” indicates that the substance or composition must be compatible chemically and/or toxicologically, with the other ingredients comprising a formulation, and/or the mammal being treated therewith.
j. “salts” is intended to refer to pharmaceutically acceptable salts and to salts suitable for use in industrial processes, such as the preparation of the compound.
k. “pharmaceutically acceptable salts” is intended to refer to either pharmaceutically acceptable acid addition salts” or “pharmaceutically acceptable basic addition salts” depending upon actual structure of the compound.
l. “pharmaceutically acceptable acid addition salts” is intended to apply to any non-toxic organic or inorganic acid addition salt of the base compounds represented by Formula I or any of its intermediates. Illustrative inorganic acids which form suitable salts include hydrochloric, hydrobromic, sulphuric, and phosphoric acid and acid metal salts such as sodium monohydrogen orthophosphate, and potassium hydrogen sulfate. Illustrative organic acids, which form suitable salts include the mono-, di-, and tricarboxylic acids. Illustrative of such acids are for example, acetic, glycolic, lactic, pyruvic, malonic, succinic, glutaric, fumaric, malic, tartaric, citric, ascorbic, maleic, hydroxymaleic, benzoic, hydroxy-benzoic, phenylacetic, cinnamic, salicylic, 2-phenoxybenzoic, p-toluenesulfonic acid, and sulfonic acids such as methane sulfonic acid and 2-hydroxyethane sulfonic acid. Such salts can exist in either a hydrated or substantially anhydrous form. In general, the acid addition salts of these compounds are soluble in water and various hydrophilic organic solvents.
m. “pharmaceutically acceptable basic addition salts” is intended to apply to any non-toxic organic or inorganic basic addition salts of the compounds represented by Formula I, or any of its intermediates. Illustrative bases which form suitable salts include alkali metal or alkaline-earth metal hydroxides such as sodium, potassium, calcium, magnesium, or barium hydroxides; ammonia, and aliphatic, alicyclic, or aromatic organic amines such as methylamine, dimethylamine, trimethylamine, and picoline.
n. “compound of Formula I”, “compounds of the invention”, and “compounds” are used interchangeably throughout the application and should be treated as synonyms.
“isomer” means “stereoisomer” and “geometric isomer” as defined below.
o. “stereoisomer” refers to compounds that possess one or more chiral centers and each center may exist in the R or S configuration. Stereoisomers includes all diastereomeric, enantiomeric and epimeric forms as well as racemates and mixtures thereof.
p. “geometric isomer” refers to compounds that may exist in cis, trans, anti, syn, entgegen (E), and zusammen (Z) forms as well as mixtures thereof.

Certain of the compounds of the formula (I) may exist as geometric isomers. The compounds of the formula (I) may possess one or more asymmetric centers, thus existing as two, or more, stereoisomeric forms. The present invention includes all the individual stereoisomers and geometric isomers of the compounds of formula (I) and mixtures thereof. Individual enantiomers can be obtained by chiral separation or using the relevant enantiomer in the synthesis.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention. The compounds may also exist in one or more crystalline states, i.e. polymorphs, or they may exist as amorphous solids. All such forms are encompassed by the claims.

Many of the compounds of Formula I contain an 3-oxa-7-azabicyclo[3.3.1]nonane ring bonded to a pyrimidine ring via an ether linkage as depicted below. This azabicyclo-nonane will exist as a geometric isomer and may be present as either the syn or anti isomer as depicted below.

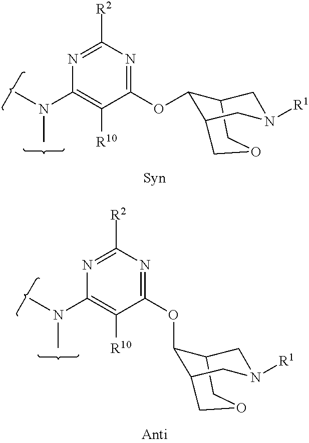

All of the compounds of Formula I contain a phenyl ring or a nitrogen containing aromatic fused to a pyrrolidine moiety as depicted below:

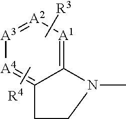

A¹-A⁴ may represent up to two nitrogen atoms and the remainder will be CH. Thus, the aromatic portion of this fused ring may represent, for example, phenyl, pyridyl, pyrimidinyl, pyridazinyl, or pyrazinyl. R³ may be hydrogen, or one of the substituents specified above. When R³ is not hydrogen, it may represent up to two substituents that may be bonded to any carbon atom of the fused ring (with the exception of the two carbons at the ring fusion (i.e. forming the fused pyrrolidinyl moiety). R⁴ may be present, or absent, and if present may be bonded to any carbon atom on the ring (with the exception of the two carbons forming the fused pyrrolidinyl moiety).

Additionally one of A¹-A⁴ may represent an N-oxide moiety. In any situation in which the aryl moiety represented by A¹-A⁴ is substituted, then the relevant carbon atom will represent CR³ or CR⁴, not CH; as is readily apparent to one skilled in the art.

Examples of such fused nitrogen containing rings include:

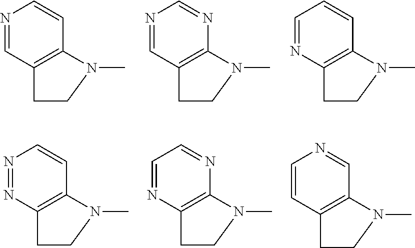

In a more specific embodiment of the invention, X is represented by a 3-oxa-7-azabicyclo[3.3.1]nonane as depicted below and the remaining variables are as defined above:

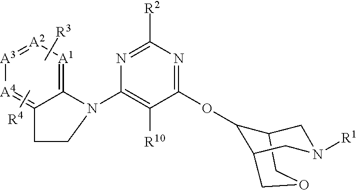

In another embodiment, X is a piperidine as represented by:

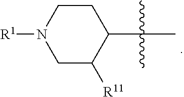

In more specific embodiments:
a) R¹ is —C(O)—O—R⁵, X is a piperidine or 3-oxa-7-azabicyclo[3.3.1]nonane, R² is hydrogen or cyano, R¹⁰ is hydrogen, A¹-A⁴ forms a phenyl ring, R³ is hydrogen and R⁴ is —SO₂—R⁷;
b) R¹ is —C(O)—O—R⁵, X is a piperidine or 3-oxa-7-azabicyclo[3.3.1]nonane, R² is hydrogen or cyano, R¹⁰ is hydrogen, A¹-A⁴ forms a phenyl ring, R³ is fluoro, R⁴ is —SO₂—R⁷; and
c) R¹ is —C(O)—O—R⁵, X is a piperidine or 3-oxa-7-azabicyclo[3.3.1]nonane, R² is hydrogen or cyano, R¹⁰ is hydrogen, A¹-A⁴ forms a phenyl ring, R³ is hydrogen, R⁴ is NH—(CH₂)₂—OH.

In another embodiment, A¹-A⁴ forms a phenyl ring.

In a further embodiment, A¹-A⁴ forms a ring in which one or two of A¹, A², A³, and A⁴ are N.

In yet another embodiment, A¹-A⁴ forms a pyridyl ring.

In another embodiment, R⁴ is absent or —CO—NR⁸R⁹.

In another embodiment, R¹ is —C(O)—O—R⁵.

In another embodiment, R³ is fluoro or hydrogen.

In another embodiment, R² is hydrogen or cyano.

### Synthesis

Compounds of the invention may be synthesized by synthetic routes that include processes analogous to those well-known in the chemical arts, particularly in light of the description contained herein. The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der omanischen Chemie, 4, Aufl. ed. Springer-Verlag, Berlin, including supplements (also available via the Beilstein online database).

For illustrative purposes, the reaction schemes depicted below provide potential routes for synthesizing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

The compounds of Formula I can be prepared using methods analogously known in the art for the production of ethers. The reader's attention is directed to texts such as: 1) Hughes, D. L.; Organic Reactions 1992, 42 Hoboken, N.J., United States; 2) Tikad, A.; Routier, S.; Akssira, M.; Leger, J.-M. I; Jarry, C.; Guillaumet, G. Synlett 2006, 12, 1938-42; and 3) Loksha, Y. M.; Globisch, D.; Pedersen, E. B.; La Colla, P.; Collu, G.; Loddo, R. J. Het. Chem. 2008, 45, 1161-6 which describe such reactions in greater detail.

Reaction Scheme I, immediately below, illustrates alternative methodologies for assembling the compounds of Formula I. The central portion of the molecule is an optionally substituted pyrimidine ring. The compounds of Formula I are produced by forming both an ether linkage and an amino linkage with the pyrimidine as depicted below. It is not critical in what order this reaction sequence is carried out.

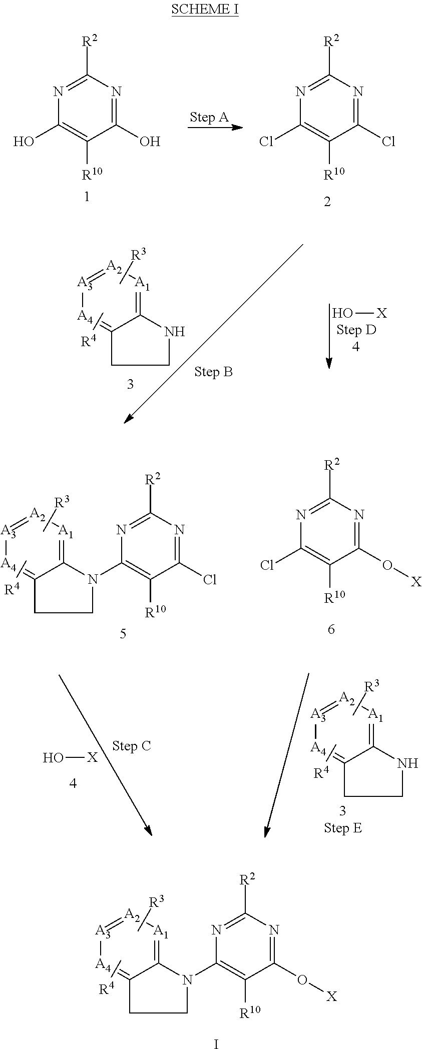

The starting material in Reaction Scheme I, is the dihydroxy-pyrimidine of structure 1 in which R² and R¹⁰ are typically represented by the same substituent as is desired in the final product. Methods for producing such pyrimidines are known in the art.

The chlorination reaction of step A is carried out as is known in the art. A compound of structure 1 is allowed to react with a chlorinating reagent such POCl₃ (phosphorous oxychloride) (Matulenko, M. A. et al., Bioorg. Med. Chem. 2007, 15, 1586-1605) to produce a dichloropyrimidine of structure 2. The chlorinating agent is used in excess or in solvents such as a toluene, benzene or xylene with or without additives such as triethylamine, N,N-dimethylaniline, or diisopropylethylamine. This reaction may be run at temperatures ranging from room temperature to 140° C., depending on the choice of conditions. Alternative chlorinating reagents include PCl₃, (phosphorous trichloride), POCl₃/PCl₅ (phosphorous pentachloride), thionyl chloride, oxalyl chloride or phosgene. In some cases the dichloropyrimidine of structure 2 may be obtained from commercial sources. Optionally, the dichloropyrimidine of structure 2 may be isolated and recovered from the reaction and further purified as is known in the art. Alternatively the crude may be used in Step B described below.

In Step B, an amino linkage is formed between the fused pyrrolidine of structure 3 and the dichloropyrimidine of structure 2. In the fused pyrrolidine of structure 3; A¹, A², A³, A⁴, R³, and R⁴ will typically be represented by the same substituent as is desired in the final product. Such pyrrolidine derivatives are known in the art and are described in: (a) Zhao, H.; Thurkauf, A.; He, X.; Hodgetts, K.; Zhang, Xi.; Rachwal, S.; Kover, R. X.; Hutchison, A.; Peterson, J.; Kieltyka, A.; Brodbeck, R.; Primus, R.; Wasley, J. W. F. Bioorg. Med. Chem. Lett. 2002, 12, 3105. (b) Nomura, S.; Yamamota, Y. WO2006080577. (c) Gribble, G.; Hoffman, J. H. Synthesis 1983, 13, 489. (d) Sassatelli, M.; Bouchikhi, F.; Messaoudi, S.; Anizon, F.; Debiton, E.; Barthomeuf, C.; Prudhomme,.; Moreau, P. Eur. J. Med. Chem. 2006, 41, 88. The examples also provide additional teachings and references to such preparations.

The amino linkage is formed by contacting equivalent amounts of the compounds of structure 2 and 3 in a polar protic solvent such as ethanol, propanol, isopropanol or butanol at temperatures ranging from 0° C. to 120° C., depending on which solvent is used, for 0.5 to 24 hours. Typical conditions utilized for this reaction are the use of isopropanol as the solvent heated at 108° C. for one hour. Alternatively, an amine base such as triethylamine or diethylisopropylamine or inorganic bases such as sodium bicarbonate, potassium carbonate or sodium carbonate may be added to this reaction. In the case of the use of one of the above amine or inorganic bases, the solvent may be changed to a polar aprotic solvent such as acetonitrile, N,N-dimethyl formamide (“DMF”), tetrahydrofuran (“THF”) or 1,4-dioxane at 0° C.-100° C. for 0.5 to 24 hours. Typical conditions utilized for this reaction include the use of diethylisopropylamine in acetonitrile at room temperature for three hours. Also, the use of hydrochloric acid in polar protic solvents such as water, methanol, ethanol or propanol alone or in combination may be used for this transformation at temperatures of 0° C. to 110° C. Typical conditions are the use of water in ethanol at 78° C. The intermediate of structure 5 may be isolated and recovered from the reaction and further purified as is known in the art. Alternatively the crude may be used in Step B described below.

In Step C, an ether linkage is formed between the intermediate of structure 5 and the alcohol of structure 4 to form the compound of Formula I. The alcohol of structure 4 will either be a 3-oxa-7-azabicyclo[3.3.1]nonanol or a hydroxy substituted piperidine, depending upon the desired final product. In these heterocyclic rings, R¹ and R¹¹, will typically be represented by the same substituent as is desired in the final product. Reaction Scheme II, hereinafter, teaches a method for the production of the 3-oxa-7-azabicyclo[3.3.1]nonanols. The hydroxyl substituted piperidines are well known in the art and are described in publications such as: (a) Gharbaoui, T.; Sengupta, D.; Lally, E. A.; Kato, N. S.; Carlos, M.; Rodriguez, N. US2006154940. (b) Wessig, P.; Moellnitz, K.; Eiserbeck, C. Chem. Eur. J. 2007, 13, 4859. (c) Kreidler, B.; Baro, A.; Christoffers, J. Eur. J. Org. Chem. 2005, 24, 5339. (d) Jingyuan, M. A.; Rabbat, C. J.; Song, J.; Chen, X.; Nashashibi, I.; Zhao, Z.; Novack, A.; Shi, D. F.; Cheng, P.; Zhu, Y.; Murphy, A.; WO2009014910. (e) Schlienger, N.; Thygesen, M. B.; Pawlas, J.; Badalassi, F.; Lewinsky, R.; Lund, B. W.; Olsson, R. WO2006076317.

In Step C, equivalent amounts of the reactants are contacted in the presence of a base such as sodium hydride; sodium and potassium tert-butoxide; sodium, potassium, and lithium bis(trimethylsilyl)amide and sodium, potassium and lithium tert-amyloxide in solvents such as DMF, THF, 1,2-dimethoxyethane, 1,4-dioxane, N,N-dimethylacetamide, or dimethylsulfoxide (“DMSO”). Typical conditions for this transformation include the use of sodium bis(trimethylsilyl)amide in dioxane at 105° C. for one hour.

After the reaction is completed the desired compound of Formula I may be recovered and isolated as known in the art. It may be recovered by evaporation, extraction, etc. as is known in the art. It may optionally be purified by chromatography, recrystallization, distillation, or other techniques known in the art prior.

As is also readily apparent to one skilled in the art, many of the substituents represented by R¹ and R⁴ may be manipulated after the core of Formula I is produced. For example, a sulfonyl moiety may be generated by oxidizing a thioether. Such variations are well known to those skilled in the art and should be considered part of the invention.

In the alternative synthesis depicted above in Reaction Scheme I, the dichloro-pyrimidine of structure 2 is initially contacted with the alcohol of structure 4 to form the intermediate depicted by structure 6. As with Step C, the alcohol of structure 4 will either be a 3-oxa-7-azabicyclo[3.3.1]nonanol or a hydroxyl-substituted piperidine, depending upon the desired final product. In these heterocyclic rings, R¹ will typically be represented by the same substituent as is desired in the final product.

Equivalent amounts of the compounds of structure 2 and structure 4 are allowed to react in the presences of a polar aprotic solvent and a base to form intermediates of structure 6 as depicted in step D. Suitable systems include bases such as sodium hydride; sodium and potassium tert-butoxide; sodium, potassium, and lithium bis(trimethylsilyl)amide and sodium, potassium and lithium tert-amyloxide in solvents such as DMF, THF, 1,2-dimethoxyethane, 1,4-dioxane, N,N-dimethylacetamide, or DMSO at temperatures of 0° C. to 140° C. Typical conditions for this transformation include the use of potassium tert-butoxide in THF at 0° C. to room temperature for 14 hours. The intermediate of structure 6 may be isolated and recovered from the reaction and further purified as is known in the art. Alternatively the crude may be used in Step E, described below.

The compounds of Formula I may then be formed by contacting the intermediate of structure 6 with the fused pyrrolidine of structure 3, previously described above. Typically, equivalent amounts of the fused pyrrolidine of structure 3 are allowed to react with the chloro intermediate of formula 6 in the presence of a base. Suitable bases can be sodium hydride; sodium or potassium tert-butoxide; sodium or potassium or lithium bis(trimethylsilyl)amide and sodium or potassium or lithium tert-amyloxide in solvents such as DMF, THF, 1,2-dimethoxyethane, 1,4-dioxane, N,N-dimethylacetamide, or DMSO or mixtures thereof. These reactions may be carried out in temperature ranges of −10° C.-150° C. depending on the solvent of use. Typically, the reaction will be allowed to proceed for a period of time ranging from 15 minutes to 24 hours under an inert atmosphere. Suitable conditions include sodium bis(dimethylsilyl)amide in dioxane at 105° C. for one hour.

Alternatively, this reaction may be carried out by heating the intermediate of structure 6 and fused pyrrolidine of structure 3 in a polar aprotic solvent such as methanol, ethanol, propanol, isopropanol or butanol for 0.5-24 hours. Typical conditions for this transformation are heating in isopropanol at 108° C. for two hours.

This reaction may also by carried out using transition metal catalysts to form the key substituted amine linkage found in the compounds of formula I. Transition metal catalysts may consist of but are not limited to Pd(PPh₃)₄, PdCl₂, Pd(OAc)₂, Pd₂(dba)₃, Cul, Cu(OAc)₂ and Cu(OTf)₂. A base is typically utilized in these reactions. A suitable base for use with palladium catalysts may be sodium tert-butoxide, potassium tert-butoxide, potassium tert-amyloxide or K₃PO₄ in an appropriate solvents such as dioxane, THF, 1,2-dimethoxyethane or toluene. For the use of copper catalysts, a suitable base may consist of alkali bases such as sodium carbonate, potassium carbonate, cesium carbonate in an appropriate solvents such as DMF, DMSO or dimethylacetamide.

Typically ligands can be added to facilitate the amine formation reaction. Ligands for palladium catalyzed reactions may include but are not limited to 9,9-Dimethyl-4,5-bis(diphenylphosphino)xanthene (Xantphos), 2,2′-bis(diphenylphosphino)-1,1′-binaphthyl (BINAP), 1,1′-Bis(diphenylphosphino)ferrocene (DPPF), 2,8,9-Triisobutyl-2,5,8,9-tetraaza-1-phosphabicyclo[3.3.3]undecane (P[N(i-Bu)CH₂CH₃]₃N), Tri-tert-butylphosphine (tBu₃P), (Biphenyl-2-yl)bis(tert-butyl)phosphine (JohnPhos), Pd-PEPPSI™-SIPr: (1,3-bis(2,6-diisopropylphenyl)-4,5-dihydroimidazol-2-ylidene) (3-chloropyridyl)palladium(II) dichloride. Suitable ligands for copper catalyzed reactions may include but are not limited to L-proline, N-methylglycine, diethylsalicylamide. Suitable conditions for formation of compounds of formula I are the use of Pd₂(dba)₃ with sodium tert-butoxide in toluene at 120° C. for 12 hours.

After the reaction is completed the desired compound of Formula I may be recovered and isolated as known in the art. It may be recovered by evaporation, extraction, etc. as is known in the art. It may optionally be purified by chromatography, recrystallization, distillation, or other techniques known in the art prior.

As is also readily apparent to one skilled in the art, many of the substituents represented by R¹ and R⁴ may be manipulated after the core of Formula I is produced. Such variations are well known to those skilled in the art and should be considered part of the invention. In many cases, compounds of formula I are substituted with R³ or R⁴ being equal to a thioalkyl (S-alkyl) moiety. This group may be oxidized to R³ or R⁴ being equal to an alkylsulfone (SO₂-alkyl) group. Utilizing an 2 to 4 equivalents of an oxidant such as meta-chloroperbenzoic acid (mCPBA) in a chlorinated solvent such as dichloromethane, chloroform or 1,2-dichloroethane is typical for this oxidation. Suitable conditions include the use of 2.7 equivalents of mCPBA in dichloromethane at room temperature for one hour.

Reaction Scheme II, immediately below, teaches a method for the production of the 3-oxa-7-azabicyclo[3.3.1]nonanols described by structure 4 above. The compound of structure 7, depicted below, is known in the art. Its synthesis is taught in Arjunan, P.; Berlin, K. D.; Barnes C. L.; Van der Helm, D. J. Org. Chem., 1981, 46, 3196-3204.

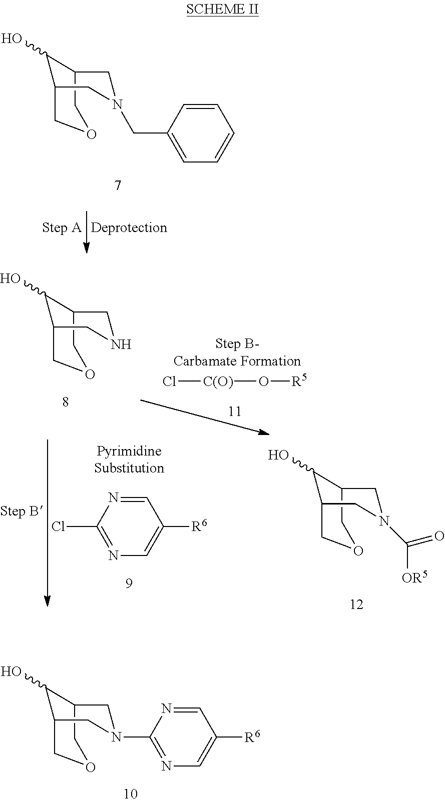

As shown above, the initial step in the reaction is to remove the benzyl protecting group from structure 7. This can be accomplished via hydrogenolysis to give compound 8. Typical conditions for this reaction include utilizing hydrogen and a palladium catalyst including 5-20% palladium on carbon or 10-20% palladium hydroxide. A typical solvent for this reaction is ethanol, methanol, tetrahydrofuran or ethyl acetate.

If a pyrimidine substituent is desired in the final product, then structure 10 may be formed via the addition of compound 8 to an appropriately substituted 2-chloropyrimidine as depicted by structure 9 in the presence of a base such as cesium carbonate or diisopropylethylamine in a protic solvent such as ethanol or methanol, or a polar aprotic solvent such as 1,4-dioxane, tetrahydrofuran, dimethylformamide or dimethylsulfoxide. These reactions can be conducted at temperatures ranging from room temperature to 110° C. Alternatively, compounds of structure 8 and structure 9 can be heated together in the presence of base such as diisopropylethylamine without solvent, or where compound 8 is used in excess without base or solvent.

If a carbamate substituent is desired in the final product then equivalent amounts the alkyl haloformate formate of structure 11 is contacted with the compound of structure 8 in the presence of a base such as diisopropylethylamine, triethylamine or pyridine in dichloromethane or chloroform. Alternatively, compounds of structure 12 can formed from compounds of structure 8 via the use of dialkyldicarbonates such as di-tert-butyl dicarbonate (BOC anhydride) or di-isopropyl dicarbonate in the presence of amine bases such as diisopropylethylamine, pyridine, 2,6-lutidine or triethylamine in solvents such as dichloromethane, chloroform or tetrahydrofuran.

Final structure 10 or 12 (i.e. structure #1 from Reaction Scheme 1) may be isolated and purified as is known in the art. If desired, it may be subjected to a separation step to yield the desired syn or anti isomer prior to its utilization in Reaction Scheme I.

As is readily apparent to one skilled in the art, protection of remote functionality (e.g., primary or secondary amine) of intermediates may be necessary. The need for such protection will vary depending on the nature of the remote functionality and the conditions of the preparation methods. Suitable amino-protecting groups (NH-Pg) include acetyl, trifluoroacetyl, t-butoxycarbonyl (BOC), benzyloxycarbonyl (CBZ) and 9-fluorenylmethyleneoxycarbonyl (Fmoc). Similarly, a “hydroxy-protecting group” refers to a substituent of a hydroxy group that blocks or protects the hydroxy functionality. Suitable hydroxyl-protecting groups (O-Pg) include for example, allyl, acetyl, silyl, benzyl, para-methoxybenzyl, trityl, and the like. The need for such protection is readily determined by one skilled in the art. For a general description of protecting groups and their use, see T. W. Greene, Protective Groups in Organic Synthesis, John Wiley & Sons, New York, 1991.

As noted above, some of the compounds of this invention are acidic and they form salts with pharmaceutically acceptable cations. Some of the compounds of this invention are basic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures

As noted above, some of the compounds exist as isomers. These isomeric mixtures can be separated into their individual isomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column. Alternatively, the specific stereoisomers may be synthesized by using an optically active starting material, by asymmetric synthesis using optically active reagents, substrates, catalysts or solvents, or by converting one stereoisomer into the other by asymmetric transformation.

The present invention also embraces isotopically-labeled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively.

Certain isotopically-labeled compounds of the present invention (e.g., those labeled with ³H and ¹⁴C) are useful in compound and/or substrate tissue distribution assays. Certain isotopically labeled ligands including tritium, ¹⁴C, ³⁵S and ¹²⁵I could be useful in radioligand binding assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C, and ¹⁸F are useful for positron emission tomography (PET) studies to examine receptor occupancy. Isotopically labeled compounds of the present invention can generally be prepared by following procedures analogous to those disclosed in the Schemes and/or in the Examples herein below, by substituting an isotopically labeled reagent for a non-isotopically labeled reagent.

Certain compounds of the present invention may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

### Medical Uses

Compounds of the present invention modulate the activity of G-protein-coupled receptor GPR119. As such, said compounds are useful for the prophylaxis and treatment of diseases, such as diabetes, in which the activity of GPR119 contributes to the pathology or symptoms of the disease. Consequently, another aspect of the present invention includes a method for the treatment of a metabolic disease and/or a metabolic-related disorder in an individual which comprises administering to the individual in need of such treatment a therapeutically effective amount of a compound of the invention, a salt of said compound or a pharmaceutical composition containing such compound. The metabolic diseases and metabolism-related disorders are selected from, but not limited to, hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations, endothelial dysfunction, hyper apo B lipoproteinemia and impaired vascular compliance. Additionally, the compounds may be used to treat neurological disorders such as Alzheimer's, schizophrenia, and impaired cognition. The compounds will also be beneficial in gastrointestinal illnesses such as inflammatory bowel disease, ulcerative colitis, Crohn's disease, irritable bowel syndrome, etc. As noted above the compounds may also be used to stimulate weight loss in obese patients, especially those afflicted with diabetes.

In accordance with the foregoing, the present invention further provides a method for preventing or ameliorating the symptoms of any of the diseases or disorders described above in a subject in need thereof, which method comprises administering to a subject a therapeutically effective amount of a compound of the present invention. Further aspects of the invention include the preparation of medicaments for the treating diabetes and its related co-morbidities.

In order to exhibit the therapeutic properties described above, the compounds need to be administered in a quantity sufficient to modulate activation of the G-protein-coupled receptor GPR119. This amount can vary depending upon the particular disease/condition being treated, the severity of the patient's disease/condition, the patient, the particular compound being administered, the route of administration, and the presence of other underlying disease states within the patient, etc. When administered systemically, the compounds typically exhibit their effect at a dosage range of from about 0.1 mg/kg/day to about 100 mg/kg/day for any of the diseases or conditions listed above. Repetitive daily administration may be desirable and will vary according to the conditions outlined above.

The compounds of the present invention may be administered by a variety of routes. They may be administered orally. The compounds may also be administered parenterally (i.e., subcutaneously, intravenously, intramuscularly, intraperitoneally, or intrathecally), rectally, or topically.

### Co-Administration

The compounds of this invention may also be used in conjunction with other pharmaceutical agents for the treatment of the diseases, conditions and/or disorders described herein. Therefore, methods of treatment that include administering compounds of the present invention in combination with other pharmaceutical agents are also provided. Suitable pharmaceutical agents that may be used in combination with the compounds of the present invention include anti-obesity agents (including appetite suppressants), anti-diabetic agents, anti-hyperglycemic agents, lipid lowering agents, and anti-hypertensive agents.

Suitable anti-diabetic agents include an acetyl-CoA carboxylase-2 (ACC-2) inhibitor, a diacylglycerol O-acyltransferase 1 (DGAT-1) inhibitor, a phosphodiesterase (PDE)-10 inhibitor, a sulfonylurea (e.g., acetohexamide, chlorpropamide, diabinese, glibenclamide, glipizide, glyburide, glimepiride, gliclazide, glipentide, gliquidone, glisolamide, tolazamide, and tolbutamide), a meglitinide, an α-amylase inhibitor (e.g., tendamistat, trestatin and AL-3688), an α-glucoside hydrolase inhibitor (e.g., acarbose), an α-glucosidase inhibitor (e.g., adiposine, camiglibose, emiglitate, miglitol, voglibose, pradimicin-Q, and salbostatin), a PPARγ agonist (e.g., balaglitazone, ciglitazone, darglitazone, englitazone, isaglitazone, pioglitazone, rosiglitazone and troglitazone), a PPAR α/γ agonist (e.g., CLX-0940, GW-1536, GW-1929, GW-2433, KRP-297, L-796449, LR-90, MK-0767 and SB-219994), a biguanide (e.g., metformin), a glucagon-like peptide 1 (GLP-1) agonist (e.g., exendin-3 and exendin-4), a protein tyrosine phosphatase-1B (PTP-1B) inhibitor (e.g., trodusquemine, hyrtiosal extract, and compounds disclosed by Zhang, S., et al., Drug Discovery Today, 12(9/10), 373-381 (2007)), SIRT-1 inhibitor (e.g., reservatrol), a dipeptidyl peptidease IV (DPP-IV) inhibitor (e.g., sitagliptin, vildagliptin, alogliptin and saxagliptin), an insulin secreatagogue, a fatty acid oxidation inhibitor, an A2 antagonist, a c-jun amino-terminal kinase (JNK) inhibitor, insulin, an insulin mimetic, a glycogen phosphorylase inhibitor, a VPAC2 receptor agonist, and a SGLT2 inhibitor (sodium dependent glucose transporter inhibitors such as dapagliflozin, etc). Preferred anti-diabetic agents are metformin and DPP-IV inhibitors (e.g., sitagliptin, vildagliptin, alogliptin and saxagliptin).

Suitable anti-obesity agents include 11β-hydroxy steroid dehydrogenase-1 (11β-HSD type 1) inhibitors, stearoyl-CoA desaturase-1 (SCD-1) inhibitor, MCR-4 agonists, cholecystokinin-A (CCK-A) agonists, monoamine reuptake inhibitors (such as sibutramine), sympathomimetic agents, β₃ adrenergic agonists, dopamine agonists (such as bromocriptine), melanocyte-stimulating hormone analogs, 5HT2c agonists, melanin concentrating hormone antagonists, leptin (the OB protein), leptin analogs, leptin agonists, galanin antagonists, lipase inhibitors (such as tetrahydrolipstatin, i.e. orlistat), anorectic agents (such as a bombesin agonist), neuropeptide-Y antagonists (e.g., NPY Y5 antagonists), PYY₃₋₃₆ (including analogs thereof), thyromimetic agents, dehydroepiandrosterone or an analog thereof, glucocorticoid agonists or antagonists, orexin antagonists, glucagon-like peptide-1 agonists, ciliary neurotrophic factors (such as Axokine™ available from Regeneron Pharmaceuticals, Inc., Tarrytown, N.Y. and Procter & Gamble Company, Cincinnati, Ohio), human agouti-related protein (AGRP) inhibitors, ghrelin antagonists, histamine 3 antagonists or inverse agonists, neuromedin U agonists, MTP/ApoB inhibitors (e.g., gut-selective MTP inhibitors, such as dirlotapide), opioid antagonist, orexin antagonist, and the like.

Preferred anti-obesity agents for use in the combination aspects of the present invention include gut-selective MTP inhibitors (e.g., dirlotapide, mitratapide and implitapide, R56918 (CAS No. 403987) and CAS No. 913541-47-6), CCKa agonists (e.g., N-benzyl-2-[4-(1H-indol-3-ylmethyl)-5-oxo-1-phenyl-4,5-dihydro-2,3,6,10b-tetraaza-benzo[e]azulen-6-yl]-N-isopropyl-acetamide described in PCT Publication No. WO 2005/116034 or US Publication No. 2005-0267100 A1), 5HT2c agonists (e.g., lorcaserin), MCR4 agonist (e.g., compounds described in U.S. Pat. No. 6,818,658), lipase inhibitor (e.g., Cetilistat), PYY₃₋₃₆ (as used herein “PYY₃₋₃₆” includes analogs, such as peglated PYY₃₋₃₆ e.g., those described in US Publication 2006/0178501), opioid antagonists (e.g., naltrexone), oleoyl-estrone (CAS No. 180003-17-2), obinepitide (TM30338), pramlintide (Symlin®), tesofensine (NS2330), leptin, liraglutide, bromocriptine, orlistat, exenatide (Byetta®), AOD-9604 (CAS No. 221231-10-3) and sibutramine. Preferably, compounds of the present invention and combination therapies are administered in conjunction with exercise and a sensible diet.

All of the above recited U.S. patents and publications are incorporated herein by reference.

### Pharmaceutical Formulations

The present invention also provides pharmaceutical compositions which comprise a therapeutically effective amount of a compound, or a pharmaceutically acceptable salt thereof, in admixture with at least one pharmaceutically acceptable excipient. The compositions include those in a form adapted for oral, topical or parenteral use and can be used for the treatment of diabetes and related conditions as described above.

The composition can be formulated for administration by any route known in the art, such as subdermal, inhalation, oral, topical, parenteral, etc. The compositions may be in any form known in the art, including but not limited to tablets, capsules, powders, granules, lozenges, or liquid preparations, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerin, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as local anesthetics, preservatives and buffering agents etc. can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% to about 99 by weight, of the active material, depending on the method of administration. Where the compositions comprise dosage units, each unit will contain, for example, from about 0.1 to 900 mg of the active ingredient, more typically from 1 mg to 250 mg.

Compounds of the invention can be formulated for administration in any convenient way for use in human or veterinary medicine, by analogy with other anti-diabetic agents. Such methods are known in the art and have been summarized above. For a more detailed discussion regarding the preparation of such formulations; the reader's attention is directed to Remington“s Pharmaceutical Sciences, 21^{st} Edition, by University of the Sciences in Philadelphia.

Embodiments of the present invention are illustrated by the following Examples. It is to be understood, however, that the embodiments of the invention are not limited to the specific details of these Examples, as other variations thereof will be known, or apparent in light of the instant disclosure, to one of ordinary skill in the art.

### EXAMPLES

Unless specified otherwise, starting materials are generally available from commercial sources such as Aldrich Chemicals Co. (Milwaukee, Wis.), Lancaster Synthesis, Inc. (Windham, N.H.), Acros Organics (Fairlawn, N.J.), Maybridge Chemical Company, Ltd. (Cornwall, England), Tyger Scientific (Princeton, N.J.), and AstraZeneca Pharmaceuticals (London, England), Mallinckrodt Baker (Phillipsburg N.J.); EMD (Gibbstown, N.J.).

### General Experimental Procedures

NMR spectra were recorded on a Varian Unity™ 400 (DG400-5 probe) or 500 (DG500-5 probe—both available from Varian Inc., Palo Alto, Calif.) at room temperature at 400 MHz or 500 MHz respectively for proton analysis. Chemical shifts are expressed in parts per million (delta) relative to residual solvent as an internal reference. The peak shapes are denoted as follows: s, singlet; d, doublet; dd, doublet of doublet; t, triplet; q, quartet; m, multiplet; bs, broad singlet; 2s, two singlets.

Atmospheric pressure chemical ionization mass spectra (APCI) were obtained on a Waters™ Spectrometer (Micromass ZMD, carrier gas: nitrogen) (available from Waters Corp., Milford, Mass., USA) with a flow rate of 0.3 mL/minute and utilizing a 50:50 water/acetonitrile eluent system. Electrospray ionization mass spectra (ES) were obtained on a liquid chromatography mass spectrometer from Waters™ (Micromass ZQ or ZMD instrument (carrier gas: nitrogen) (Waters Corp., Milford, Mass., USA) utilizing a gradient of 95:5-0:100 water in acetonitrile with 0.01% formic acid added to each solvent. These instruments utilized a Varian Polaris 5 C18-A20×2.0 mm column (Varian Inc., Palo Alto, Calif.) at flow rates of 1 mL/minute for 3.75 minutes or 2 mL/minute for 1.95 minutes.

Column chromatography was performed using silica gel with either Flash 40 Biotage™ columns (ISC, Inc., Shelton, Conn.) or Biotage™ SNAP cartridge KPsil or Redisep Rf silica (from Teledyne Isco Inc) under nitrogen pressure. Preparative HPLC was performed using a Waters Fraction Lynx system with photodiode array (Waters 2996) and mass spectrometer (Waters/Micromass ZQ) detection schemes. Analytical HPLC work was conducted with a Waters 2795 Alliance HPLC or a Waters ACQUITY HPLC with photodiode array, single quadrupole mass and evaporative light scattering detection schemes.

Concentration in vacuo refers to evaporation of solvent under reduced pressure using a rotary evaporator.

Unless otherwise noted, chemical reactions were performed at room temperature (about 23 degrees Celsius). Also, unless otherwise noted chemical reactions were run under an atmosphere of nitrogen.

### Pharmacological Data

The practice of the invention for the treatment of diseases modulated by the agonist activation of the G-protein-coupled receptor GPR119 with compounds of the invention can be evidenced by activity in one or more of the functional assays described herein below. The source of supply is provided in parenthesis.

### In-Vitro Functional Assays

β-lactamase:

The assay for GPR119 agonists utilizes a cell-based (hGPR119HEK293-CRE beta-lactamase) reporter construct where agonist activation of human GPR119 is coupled to beta-lactamase production via a cyclic AMP response element (CRE). GPR119 activity is then measured utilizing a FRET-enabled beta-lactamase substrate, CCF4-AM (Live Blazer FRET-B/G Loading kit, Invitrogen cat #K1027). Specifically, hGPR119-HEK-CRE-beta-lactamase cells (Invitrogen 2.5×10⁷/mL) were removed from liquid nitrogen storage, and diluted in plating medium (Dulbecco's modified Eagle medium high glucose (DMEM; Gibco Cat #11995-065), 10% heat inactivated fetal bovine serum (HIFBS; Sigma Cat # F4135), 1×MEM Nonessential amino acids (Gibco Cat #15630-080), 25 mM HEPES pH 7.0 (Gibco Cat #15630-080), 200 nM potassium clavulanate (Sigma Cat # P3494). The cell concentration was adjusted using cell plating medium and 50 microL of this cell suspension (12.5×10⁴ viable cells) was added into each well of a black, clear bottom, poly-d-lysine coated 384-well plate (Greiner Bio-One cat #781946) and incubated at 37 degrees Celsius in a humidified environment containing 5% carbon dioxide. After 4 hours the plating medium was removed and replaced with 40 microL of assay medium (Assay medium is plating medium without potassium clavulanate and HIFBS). Varying concentrations of each compound to be tested was then added in a volume of 10 microL (final DMSO 0.5%) and the cells were incubated for 16 hours at 37 degrees Celsius in a humidified environment containing 5% carbon dioxide. Plates were removed from the incubator and allowed to equilibrate to room temperature for approximately 15 minutes. 10 microL of 6×CCF4/AM working dye solution (prepared according to instructions in the Live Blazer FRET-B/G Loading kit, Invitrogen cat # K1027) was added per well and incubated at room temperature for 2 hours in the dark. Fluorescence was measured on an EnVision fluorimetric plate reader, excitation 405 nm, emission 460 nm/535 nm. EC₅₀ determinations were made from agonist-response curves analyzed with a curve fitting program using a 4-parameter logistic dose-response equation.

cAMP:

GPR119 agonist activity was also determined with a cell-based assay utilizing an HTRF (Homogeneous Time-Resolved Fluorescence) cAMP detection kit (cAMP dynamic 2 Assay Kit; Cis Bio cat #62AM4PEC) that measures cAMP levels in the cell. The method is a competitive immunoassay between native cAMP produced by the cells and the cAMP labeled with the dye d2. The tracer binding is visualized by a Mab anti-cAMP labeled with Cryptate. The specific signal (i.e. energy transfer) is inversely proportional to the concentration of cAMP in either standard or sample.

Specifically, hGPR119HEK-CRE beta-lactamase cells (Invitrogen 2.5×10⁷/mL; the same cell line used in the beta-lactamase assay described above) are removed from cryopreservation and diluted in growth medium (Dulbecco's modified Eagle medium high glucose (DMEM; Gibco Cat #11995-065), 1% charcoal dextran treated fetal bovine serum (CD serum; HyClone Cat # SH30068.03), 1×MEM Nonessential amino acids (Gibco Cat #15630-080) and 25 mM HEPES pH 7.0 (Gibco Cat #15630-080)). The cell concentration was adjusted to 1.5×10⁵ cells/mL and 30 mLs of this suspension was added to a T-175 flask and incubated at 37 degrees Celsius in a humidified environment in 5% carbon dioxide. After 16 hours (overnight), the cells were removed from the T-175 flask (by rapping the side of the flask), centrifuged at 800×g and then re-suspended in assay medium (1×HBSS+CaCl₂+MgCl₂ (Gibco Cat #14025-092) and 25 mM HEPES pH 7.0 (Gibco Cat #15630-080)). The cell concentration was adjusted to 6.25×10⁵ cells/mL with assay medium and 8 μl of this cell suspension (5000 cells) was added to each well of a white Greiner 384-well, low-volume assay plate (VWR cat #82051-458).

Varying concentrations of each compound to be tested were diluted in assay buffer containing 3-isobutyl-1-methylxanthin (IBMX; Sigma cat #15879) and added to the assay plate wells in a volume of 2 microL (final IBMX concentration was 400 microM and final DMSO concentration was 0.58%). Following 30 minutes incubation at room temperature, 5 microL of labeled d2 cAMP and 5 microL of anti-cAMP antibody (both diluted 1:20 in cell lysis buffer; as described in the manufacturers assay protocol) were added to each well of the assay plate. The plates were then incubated at room temperature and after 60 minutes, changes in the HTRF signal were read with an Envision 2104 multilabel plate reader using excitation of 330 nm and emissions of 615 and 665 nm. Raw data were converted to nM cAMP by interpolation from a cAMP standard curve (as described in the manufacturer's assay protocol) and EC50 determinations were made from an agonist-response curves analyzed with a curve fitting program using a 4-paramter logistic dose response equation.

It is recognized that cAMP responses due to activation of GPR119 could be generated in cells other than the specific cell line used herein.

### β-Arrestin:

GPR119 agonist activity was also determined with a cell-based assay utilizing DiscoverX PathHunter β-arrestin cell assay technology and their U2OS hGPR119 β-arrestin cell line (DiscoverX Cat #93-0356 C3). In this assay, agonist activation is determined by measuring agonist-induced interaction of β-arrestin with activated GPR119. A small, 42 amino acid enzyme fragment, called ProLink was appended to the C-terminus of GPR119. Arrestin was fused to the larger enzyme fragment, termed EA (Enzyme Acceptor). Activation of GPR119 stimulates binding of arrestin and forces the complementation of the two enzyme fragments, resulting in formation of a functional β-galactosidase enzyme capable of hydrolyzing substrate and generating a chemiluminescent signal.

Specifically, U2OS hGPR119β-arrestin cells (DiscoverX 1×10⁷/mL) are removed from cryopreservation and diluted in growth medium (Minimum essential medium (MEM; Gibco Cat #11095-080), 10% heat inactivated fetal bovine serum (HIFBS; Sigma Cat # F4135-100), 100 mM sodium pyruvate (Sigma Cat # S8636), 500 microg/mL G418 (Sigma Cat # G8168) and 250 microg/mL Hygromycin B (Invitrogen Cat #10687-010). The cell concentration was adjusted to 1.66×10⁵ cells/mL and 30 mLs of this suspension was added to a T-175 flask and incubated at 37 degrees Celsius in a humidified environment in 5% carbon dioxide. After 24 hours, the cells were removed from the T-175 flask with enzyme-free cell dissociation buffer (Gibco cat #13151-014), centrifuged at 800×g and then re-suspended in plating medium (Opti-MEM I (Invitrogen/BRL Cat #31985-070) and 2% charcoal dextran treated fetal bovine serum (CD serum; HyClone Cat # SH30068.03). The cell concentration was adjusted to 2.5×10⁵ cells/mL with plating medium and 20 microliters of this cell suspension (5000 cells) was added to each well of a white Greiner 384-well low volume assay plate (VWR cat #82051-458) and the plates were incubated at 37 degrees Celsius in a humidified environment in 5% carbon dioxide.

After 16 hours (overnight) the assay plates were removed from the incubator and varying concentrations of each compound to be tested (diluted in assay buffer (1×HBSS+CaCl₂+MgCl₂ (Gibco Cat #14025-092), 20 mM HEPES pH 7.0 (Gibco Cat #15630-080) and 0.1% BSA (Sigma Cat # A9576)) were added to the assay plate wells in a volume of 5 microliters (final DMSO concentration was 0.5%). After a 90 minute incubation at 37 degrees Celsius in a humidified environment in 5% carbon dioxide, 12 microliters of Galacton Star β-galactosidase substrate (PathHunter Detection Kit (DiscoveRx Cat #93-0001); prepared as described in the manufacturers assay protocol) was added to each well of the assay plate. The plates were incubated at room temperature and after 60 minutes, changes in the luminescence were read with an Envision 2104 multilabel plate reader at 0.1 seconds per well. EC50 determinations were made from an agonist-response curves analyzed with a curve fitting program using a 4-parameter logistic dose response equation.

### Expression of GPR119 Using BacMam and GPR119 Binding Assay

Wild-type human GPR119 (FIG. 1) was amplified via polymerase chain reaction (PCR) (Pfu Turbo Mater Mix, Stratagene, La Jolla, Calif.) using pIRES-puro-hGPR119 as a template and the following primers:

hGPR119 BamH1, Upper
5′-TAAATTGGATCCACCATGGAATCATCTTTCTCATTTGGAG-3′
(inserts a BamHI site at the 5′ end)
hGPR119 EcoRI, Lower
5′-TAAATTGAATTCTTATCAGCCATCAAACTCTGAGC-3′
(inserts a EcoRI site at the 3′ end)

The amplified product was purified (Qiaquick Kit, Qiagen, Valencia, Calif.) and digested with BamH1 and EcoRI (New England BioLabs, Ipswich, Mass.) according to the manufacturer's protocols. The vector pFB-VSVG-CMV-poly (FIG. 2) was digested with BamH1 and EcoRI (New England BioLabs, Ipswich, Mass.). The digested DNA was separated by electrophoresis on a 1% agarose gel; the fragments were excised from the gel and purified (Qiaquick Kit, Qiagen, Valencia, Calif.). The vector and gene fragments were ligated (Rapid Ligase Kit, Roche, Pleasanton, Calif.) and transformed into OneShot DH5alpha T1 R cells (Invitrogen, Carlsbad, Calif.). Eight ampicillin-resistant colonies (“clones 1-8”) were grown for miniprep (Qiagen Miniprep Kit, Qiagen, Valencia, Calif.) and sequenced to confirm identity and correct insert orientation.

The pFB-VSVG-CMV-poly-hGPR119 construct (clone #1) was transformed into OneShot DH10Bac cells (Invitrogen, Carlsbad, Calif.) according to manufacturers' protocols. Eight positive (i.e. white) colonies were re-streaked to confirm as “positives” and subsequently grown for bacmid isolation. The recombinant hGPR119 bacmid was isolated via a modified Alkaline Lysis procedure using the buffers from a Qiagen Miniprep Kit (Qiagen, Valencia, Calif.). Briefly, pelleted cells were lysed in buffer P1, neutralized in buffer P2, and precipitated with buffer N3. Precipitate was pelleted via centrifugation (17,900×g for 10 minutes) and the supernatant was combined with isopropanol to precipitate the DNA. The DNA was pelleted via centrifugation (17,900×g for 30 minutes), washed once with 70% ethanol, and resuspended in 50 microliters buffer EB (Tris-HCL, pH 8.5). Polymerase chain reaction (PCR) with commercially available primers (M13 F, M13R, Invitrogen, Carlsbad, Calif.) was used to confirm the presence of the hGPR119 insert in the Bacmid.

Generation of hGPR119Recombinant Baculovirus

### Creation of P0 Virus Stock

Suspension adapted Sf9 cells grown in Sf900II medium (Invitrogen, Carlsbad, Calif.) were transfected with 10 microL hGPR119 bacmid DNA according to the manufacturer's protocol (Cellfectin, Invitrogen, Carlsbad, Calif.). After five days of incubation, the conditioned medium (i.e. “P0” virus stock) was centrifuged and filtered through a 0.22 μm filter (Steriflip, Millipore, Billerica, Mass.).

### Creation of Frozen Virus (BIIC) Stocks

For long term virus storage and generation of working (i.e. “P1”) viral stocks, frozen BIIC (Baculovirus Infected Insect Cells) stocks were created as follows: suspension adapted Sf9 cells were grown in Sf900II medium (Invitrogen, Carlsbad, Calif.) and infected with hGPR119 P0 virus stock. After 24 hours of growth, the infected cells were gently centrifuged (approximately 100×g), resuspended in Freezing Medium (10% DMSO, 1% Albumin in Sf900II medium) to a final density of 1×10⁷ cells/mL and frozen according to standard freezing protocols in 1 mL aliquots.

### Creation of Working (“P1”) Virus Stock

Suspension adapted Sf9 cells grown in Sf900II medium (Invitrogen, Carlsbad, Calif.) were infected with a 1:100 dilution of a thawed hGPR119BIIC stock and incubated for several days (27 degrees Celsius with shaking). When the viability of the cells reached 70%, the conditioned medium was harvested by centrifugation and the virus titer determined by ELISA (BaculoElisa Kit, Clontech, Mountain View, Calif.)

Over-Expression of hGPR119 in Suspension-Adapted HEK 293Ft Cells

HEK 293FT cells (Invitrogen, Carlsbad, Calif.) were grown in a shake flask in 293Freestyle medium (Invitrogen) supplemented with 50 microg/mL neomycin and 10 mM HEPES (37 C, 8% carbon dioxide, shaking). The cells were centrifuged gently (approximately 500×g, 10 minutes) and the pellet resuspended in a mixture of Dulbecco's PBS(minus Mg++/−Ca++) supplemented with 18% fetal bovine serum (Sigma Aldrich) and P1 virus such that the multiplicity of infection (MOI) was 10 and the final cell density was 1.3×10⁶/mL (total volume 2.5 liters). The cells were transferred to a 5 liter Wave Bioreactor Wavebag (Wave Technologies, MA) and incubated for 4 hours at 27 degrees Celsius (17 rocks/min, 7 degrees platform angle); at the end of the incubation period, an equal volume(2.5 liters) of 293Freestyle medium supplemented with 30 mM sodium butyrate (Sigma Aldrich) was added (final concentration=15 mM), and the cells were grown for 20 hours (37 degrees Celsius, 8% CO2 [0.2 liters/min}, 25 rocks/minute, 7 degrees platform angle). Cells were harvested via centrifugation (3,000×g, 10 minutes), washed once on DPBS (minus Ca++/Mg++), resuspended in 0.25M sucrose, 25 mM HEPES, 0.5 mM EDTA, pH 7.4 and frozen at −80 degrees Celsius.

### Membrane Preparation for Radioligand Binding Assays

The frozen cells were thawed on ice and centrifuged at 700×g (1400 rpm) for 10 minutes at 4 degrees Celsius. The cell pellet was resuspended in 20 mL phosphate-buffered saline, and centrifuged at 1400 rpm for 10 minutes. The cell pellet was then resuspended in homogenization buffer (10 mM HEPES (Gibco #15630), pH 7.5, 1 mM EDTA (BioSolutions, #BIO260-15), 1 mM EGTA (Sigma, #E-4378), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) and incubated on ice for 10 minutes. Cells were then lysed with 15 gentle strokes of a tight-fitting glass Dounce homogenizer. The homogenate was centrifuged at 1000×g (2200 rpm) for 10 minutes at 4 degrees Celsius. The supernatant was transferred into fresh centrifuge tubes on ice. The cell pellet was resuspended in homogenization buffer, and centrifuged again at 1000×g (2200 rpm) for 10 minutes at 4 degrees Celsius after which the supernatant was removed and the pellet resuspended in homogenization buffer. This process was repeated a third time, after which the supernatants were combined, Benzonase (Novagen #71206) and MgCl₂ (Fluka #63020) were added to final concentrations of 1 U/mL and 6 mM, respectively, and incubated on ice for one hour. The solution was then centrifuged at 25,000×g (15000 rpm) for 20 minutes at 4 degrees Celsius, the supernatant was discarded, and the pellet was resuspended in fresh homogenization buffer (minus Benzonase and MgCl₂). After repeating the 25,000×g centrifugation step, the final membrane pellet was resuspended in homogenization buffer and frozen at −80 degrees Celsius. The protein concentration was determined using the Pierce BCA protein assay kit (Pierce reagents A #23223 and B #23224).

### Synthesis and Purification of [³H]-Compound A

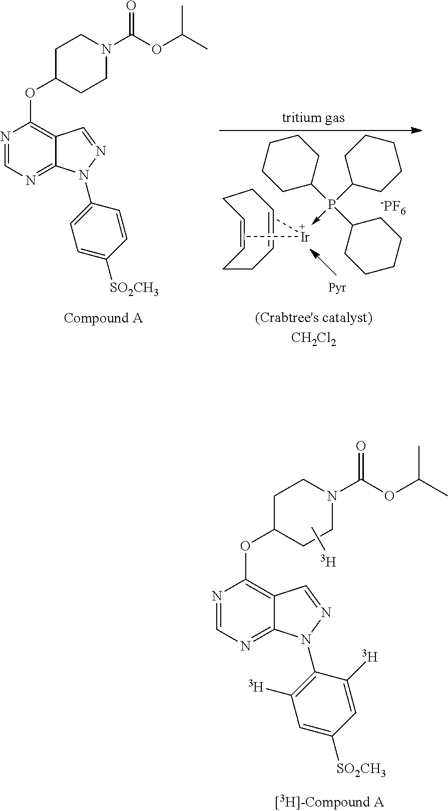

Compound A (isopropyl 4-(1-(4-(methylsulfonyl)phenyl)-3a,7a-dihydro-1H-pyrazolo[3,4-d]pyrimidin-4-yloxy)piperidine-1-carboxylate, as shown above) (4 mg, 0.009 mmol) was dissolved in 0.5 mL of dichloromethane, and the resulting solution was treated with (1,5-cyclooctadiene)(pyridine)(tricyclohexylphosphine)-iridium(I) hexafluorophosphate (J. Organometal. Chem. 1979, 168, 183) (5 mg, 0.006 mmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound A was performed by preparative HPLC using the following conditions.

### Column: Atlantis, 4.6×150 mm, 5 μm

Mobil Phase A: water/acetonitrile/formic acid (98/2/0.1)
Mobil Phase B: acetonitrile

### Gradient:

| Time | % B |
|---|---|
| 0.00 | 30.0 |
| 1.00 | 30.0 |
| 13.00 | 80.0 |

Run time: 16 min
Post time: 5 min
Flow Rate: 1.5 mL/minute
Inj. Volume: 20˜50 μL
Inj. Solvent: DMSO

### Detection: UV at 210 nm and 245 nm

The specific activity of purified [³H]-Compound A was determined by mass spectroscopy to be 70 Ci/mmol.
Alternatively the binding assay can be performed with [³H]-Compound B.

### Synthesis and Purification of [³H]-Compound B

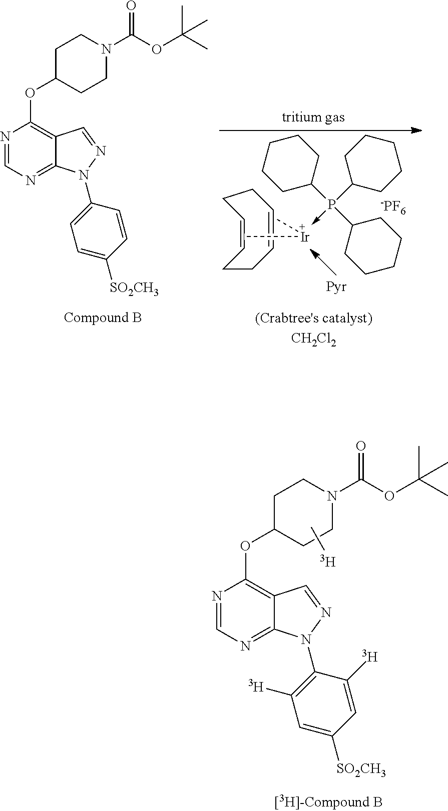

Compound B (tert-butyl 4-(1-(4-(methylsulfonyl)phenyl)-1H-pyrazolo[3,4-d]pyrimidin-4-yloxy)piperidine-1-carboxylate, as shown above) (5 mg, 10.6 μmol) was dissolved in 1.0 mL of dichloromethane and the resulting solution was treated with Crabtree's catalyst (5 mg, 6.2 mmol). The reaction vessel was sealed and the solution was stirred under an atmosphere of tritium gas for 17 hours. The reaction solvent was removed under reduced pressure and the resulting residue was dissolved in ethanol. Purification of crude [³H]-Compound B was performed by silica gel flash column chromatography eluting with 70% hexanes/30% ethyl acetate, followed by silica gel flash column chromatography eluting with 60% petroleum ether/40% ethyl acetate.

The specific activity of purified [³H]-Compound B was determined by mass spectroscopy to be 57.8 Ci/mmol.

### GPR 119 Radioligand Binding Assay

Test compounds were serially diluted in 100% DMSO (J. T. Baker #922401). 2 microL of each dilution was added to appropriate wells of a 96-well plate (each concentration in triplicate). Unlabeled Compound A (or Compound B), at a final concentration of 10 microM, was used to determine non-specific binding. [³H]-Compound A (or [³H]-Compound B) was diluted in binding buffer (50 mM Tris-HCl, pH 7.5, (Sigma #T7443), 10 mM MgCl₂ (Fluka 63020), 1 mM EDTA (BioSolutions #BIO260-15), 0.15% bovine serum albumin (Sigma #A7511), 0.01 mg/mL benzamidine (Sigma #B 6506), 0.01 mg/mL bacitracin (Sigma #B 0125), 0.005 mg/mL leupeptin (Sigma #L 8511), 0.005 mg/mL aprotinin (Sigma #A 1153)) to a concentration of 60 nM, and 100 microL added to all wells of 96-well plate (Nalge Nunc #267245).

Membranes expressing GPR119 were thawed and diluted to a final concentration of 20 μg/100 microL per well in Binding Buffer, and 100 microL of diluted membranes were added to each well of 96-well plate.

The plate was incubated for 60 minutes w/shaking at room temperature (approximately 25 degrees Celsius). The assay was terminated by vacuum filtration onto GF/C filter plates (Packard #6005174) presoaked in 0.3% polyethylenamine, using a Packard harvester. Filters were then washed six times using washing buffer (50 mM Tris-HCl, pH 7.5 kept at 4 degrees Celsius). The filter plates were then air-dyed at room temperature overnight. 30μιχρογιτερ of scintillation fluid (Ready Safe, Beckman Coulter #141349) was added to each well, plates were sealed, and radioactivity associated with each filter was measured using a Wallac Trilux MicroBeta, plate-based scintillation counter.

The Kd for [³H]-Compound A (or [³H]-Compound B) was determined by carrying out saturation binding, with data analysis by non-linear regression, fit to a one-site hyperbola (Graph Pad Prism). IC₅₀ determinations were made from competition curves, analyzed with a proprietary curve fitting program (SIGHTS) and a 4-parameter logistic dose response equation. Ki values were calculated from IC₅₀ values, using the Cheng-Prusoff equation.

The following results were obtained for the assays described above:

| | Human B- | | Human | | Human B- | | |
|---|---|---|---|---|---|---|---|
| | lactamase | | cAMP | | arrestin | | |
| | Functional | Intrinsic | Functional | Intrinsic | Functional | Intrinsic | Human |
| Example | EC50 | Activity* | EC50 | Activity* | EC50 | Activity* | Binding |
| number | (nM) | (%) | (nM) | (%) | (nM) | (%) | Ki (nM) |
| 1 | 75.9 | 107 | 339 | 96.5 | | | 219 |
| | 72.3 | 109 | 567 | 94 | | | 35.5 |
| | | | 1280 | 87.9 | | | |
| 2 | 51.3 | 106 | | | | | |
| | 38.2 | 109 | | | | | |
| | 343 | 98 | | | | | |
| 3 | 12.6 | 102 | 28.8 | 107 | 22.1 | 75.8 | 5.8 |
| | 29.7 | 81.8 | 126 | 101 | 14.9 | 62.2 | 18.6 |
| | 13.8 | 93.4 | 225 | 105 | 72.2 | 87.9 | 12.3 |
| | 12.3 | 107 | 199 | 107 | | | 8.78 |
| | | | 49.4 | 106 | | | 18.1 |
| | | | 152 | 84 | | | 29.1 |
| | | | 63.7 | 62.5 | | | 146 |
| | | | 202 | 104 | | | |
| | | | 201 | 91 | | | |
| | | | 104 | 89.4 | | | |
| 4 | 9.16 | 87.9 | 128 | 36.2 | | | 5.23 |
| | | | 624 | 36 | | | 53.7 |
| | | | 560 | 35.3 | | | 24.1 |
| | | | >10000 | | | | |
| | | | >10000 | | | | |
| | | | >10000 | | | | |
| 5 | 11.5 | 98.1 | 357 | 103 | | | 44.8 |
| | 12.5 | 100 | 602 | 98.6 | | | 13.1 |
| | | | 1750 | 100 | | | 5.3 |
| | | | 1330 | 82 | | | 40.9 |
| | | | 1800 | 98.1 | | | 63.4 |
| | | | 3140 | 100 | | | |
| 6 | 7.8 | 99.9 | 84.5 | 95.3 | 4.76 | 70 | 1.87 |
| | 6.45 | 103 | 116 | 94.2 | | | 5.02 |
| 7 | 96.6 | 101 | | | | | 285 |
| 8 | 43.5 | 100 | 43.9 | 53 | | | 273 |
| | 57.4 | 99.2 | | | | | 338 |
| 9 | 37.3 | 100 | 70.9 | 97.5 | | | 58.8 |
| | 32.8 | 101 | | | | | 45.2 |
| 10 | 46.7 | 90 | 13.8 | 104 | | | 232 |
| | 37.2 | 85.8 | 32.9 | 105 | | | |
| 11 | 40.7 | 103 | 99.4 | 90.3 | | | 72.8 |
| 12 | 2100 | 110 | 8150 | 100** | 2570 | 100** | 6100 |
| | 2160 | 121 | 9620 | 100** | | | 1350 |
| | | | 8350 | 100** | | | |
| | | | 784 | 56 | | | 2910 |
| | | | 1140 | 56.5 | | | |
| | | | 1180 | 53.6 | | | |
| | | | 9480 | 100** | | | |
| | | | 331 | 44 | | | |
| | | | 792 | 56.5 | | | |
| 13 | 236 | 92.6 | 24.1 | 20.3 | 89.4 | 29.1 | 55.3 |
| | 203 | 97.8 | 39.7 | 19.5 | | | 16 |
| | | | 76.3 | 29.2 | | | 91.1 |
| | | | 30.2 | 24.4 | | | 23.8 |
| 14 | 7270 | 98.1 | >10000 | | | | |
| 15 | 7.2 | 107 | 10.3 | 92.5 | 0.999 | 77.1 | 1.72 |
| | 4.09 | 113 | 12.9 | 102 | | | 6.77 |
| | 2.82 | 112 | 49.5 | 93.5 | | | 1.6 |
| 16 | | | 6.37 | 24.8 | 2.78 | 48.6 | 1.48 |
| | | | 15 | 21.8 | | | 2.12 |
| | | | 9.29 | 27.7 | | | |
| 17 | | | 19 | 69.6 | 2.13 | 68.9 | 2.0 |
| | | | 28.5 | 48.7 | 5.86 | 54.3 | 5.04 |
| | | | 13.7 | 53.8 | 6.62 | 59.2 | 2.43 |
| | | | 14.8 | 63.9 | 5.05 | 52.8 | 4.23 |
| | | | 16.9 | 53.1 | 4.7 | 76.9 | 1.7 |
| | | | 19.4 | 48.5 | | | 5.67 |
| | | | | | | | 13.8 |
| | | | | | | | 11.9 |
| | | | | | | | 2.45 |
| | | | | | | | 6.9 |
| 18 | | | 18.8 | 71.2 | 2.57 | 75.8 | 1.27 |
| | | | 18.4 | 49.2 | 19 | 73.7 | 6.15 |
| | | | 15.9 | 54.5 | 4.94 | 81 | 4.27 |
| | | | 12.7 | 61.7 | 20.6 | 78.9 | 8.34 |
| | | | 14.6 | 57.7 | | | 5.01 |
| | | | | | | | 9.55 |
| | | | | | | | 12.3 |
| | | | | | | | 9.56 |
| 19 | | | 19.4 | 57.9 | 6.31 | 74 | |
| 20 | | | 493 | 81.5 | | | 1710 |
| | | | 765 | 91.6 | | | |
| | | | 1050 | 98 | | | |
| 21 | | | 107 | 49.8 | | | 26.0 |
| | | | 87.8 | 45.2 | | | 25.6 |
| 22 | | | 116 | 80.4 | 40.2 | 68.8 | 66.6 |
| | | | 169 | 66.6 | 33.1 | 70.5 | 79.3 |
| | | | 137 | 74.1 | 74.6 | 71.2 | 53 |
| | | | 269 | 55.6 | 41.8 | 60.3 | 96.4 |
| 23 | 64.7 | 97.9 | 311 | 26.3 | 37 | 51.6 | 130 |
| | | | 982 | 27.9 | | | |
| | | | 1040 | 29.5 | | | |
| | | | 1330 | 16.7 | | | |
| 24 | 111 | 100 | | | | | |
| 25 | | | 1400 | 68.3 | 12.4 | 81 | 39 |
| | | | 131 | 67.7 | 19.3 | 78.6 | 34.7 |
| | | | 152 | 74.3 | 40.7 | 68.6 | |
| | | | 151 | 62.9 | 18.2 | 74.5 | |
| | | | 172 | 52.9 | 14.7 | 58.7 | |
| 26 | | | 96.9 | 65.1 | 23.8 | 68.6 | 40.5 |
| | | | 112 | 67.6 | 18.2 | 82.5 | 22.2 |
| | | | 91.1 | 43.6 | 21.3 | 68.9 | |
| | | | 163 | 50 | 26.3 | 73.4 | |
| 27 | | | 74.5 | 39.1 | 4.02 | 63.5 | 20.8 |
| | | | 68.9 | 43.5 | 22.9 | 64.2 | 13.8 |
| | | | 42.7 | 30.6 | 5.81 | 58.1 | |
| | | | 69.8 | 28.4 | 11 | 51.4 | |
| 28 | | | 370 | 41.9 | 3.79 | 32.1 | 45.8 |
| | | | 194 | 37 | | | |
| | | | 88 | 36 | | | |
| 29 | | | >10000 | | >10000 | | 73.8 |
| | | | >10000 | | | | |
| 30 | | | 549 | 28 | | | >8150 |
| | | | 520 | 28 | | | |
| 31 | | | 4820 | 100** | | | 618 |
| | | | 5890 | 100** | | | |
| | | | 6100 | 100** | | | |
| 32 | | | 273 | 147 | | | |
| 33 | | | 1550 | 154 | | | |

*The intrinsic activity is the percent of maximal activity of the test compound, relative to the activity of a standard GPR119 agonist, 4-[[6-[(2-fluoro-4 methylsulfonylphenyl) amino]pyrimidin-4-yl]oxy]piperidine-1-carboxylic acid isopropyl ester (WO2005121121), at a final concentration of 10 micromolar.
**the curve was extrapolated to 100% to calculate an EC50.

### In Vivo Pharmacology

All in vivo protocols were approved by the Pfizer's Animal Welfare Committee. Naive male Wistar rats (225-250 g body weight on receipt) were obtained from Harlan Laboratories (Indianapolis, Ind.), were pair housed in hanging plastic caging on Sani-chips sawdust bedding, and fed ad libitum on Purina 5001 chow. The rats were housed under a controlled light cycle (light from 6 am to 6 pm) at controlled temperature and humidity conditions. Rats were acclimated to the facility for at least 1 week prior to study.

### Compound Preparation

Example 17 was formulated in 0.5% methylcellulose. The highest dose (30 mg/kg) was formulated at 6 mg/mL for administration at 5 mL/kg, the required bulk was added to a mortar and ground with a small amount of 0.5% methylcellulose to a smooth paste with a pestle, additional 0.5% methylcellulose was added until the mixture flowed, when it was transferred to a stirred container, the mortar was rinsed several times with remaining quantity of 0.5% methylcellulose and capped to prevent evaporation. The suspension was stirred continuously overnight with a magnetic stir bar prior to study, and the lower doses were diluted from the 6 mg/mL suspension using the appropriate volume of 0.5% methylcellulose. All dosing suspensions were stirred throughout the dosing procedure.

### Oral Glucose Tolerance Test (OGTT) Protocol

Rats were stratified (n=8/group) to vehicle (0.5% methylcellulose) or one of three dose groups (1, 5, or 30 mg/kg) according to body weight on day-1 to ensure that each group had equal group mean body weight values. The rats were fasted overnight in clean cages overnight (˜15 hours) prior to the oral glucose tolerance test. Body weights were recorded on the morning of the study (post fasting) for dose volume calculation. Blood samples were collected from all rats prior to dosing with vehicle or test compound via oral gavage (5 mL/kg). Thirty minutes later rats were bled and immediately dose with an oral dose of glucose (2 g/kg). The rats were re-bled at 15, 30, 60 and 120 minutes post-glucose load. Blood samples (˜250 μL/time point) were collected into EDTA tubes with aprotinin/DPPIVi (0.6 TIU/20 μL per mL whole blood). Blood tubes were inverted several times immediately following collection and placed on ice, then spun at 14,000 rpm in a refrigerated centrifuge for 5 minutes. Plasma samples were analyzed for glucose levels using a Roche 912 clinical chemistry analyzer, plasma insulin concentrations were determined using the Alpco Ultra-Sensitive Insulin Rat ELISA, and total amide GLP-1 concentrations were determined using MSD ELISA kit.

The results are presented as mean +/−SEM (standard error of the mean) unless otherwise stated. Statistical evaluation of the data is carried out using one-way analysis of variance (ANOVA) with appropriate post-hoc analysis between vehicle and treatment group. Differences compared to vehicle with a p>0.05 were considered statistically significant using Dunnett's T-test.

TABLE 1
Effect of Example 17 during OGTT

| | | | Total |
|---|---|---|---|
| | Glucose | | Amide GLP-1 |
| | 0-120 min | Insulin 0-60 min | 0-120 min AUC |
| Dose | AUC (percent | AUC (percent | (percent vehicle |
| (Example 17) | vehicle response) | vehicle response) | response) |
| 1 mg/kg | 102 | 90 | 147 |
| 5 mg/kg | 95 | 142 | + |
| | | | 184 |
| 30 mg/kg | ++ | 112 | 150 |
| | 90 | | |

+ p > 0.05 compared to vehicle
++ p > 0.01 compared to vehicle

### Intraperitoneal Glucose Tolerance Test (IPGTT) Protocol

Rats were assigned (n=8/group) to vehicle or one of three dose groups (1 or 10 mg/kg) according to body weight on day-1 to ensure that each group had equal group mean body weight values. The rats were fasted overnight in clean cages overnight (˜15 hours) prior to the intra-peritoneal glucose tolerance test. Body weights were recorded on the morning of the study (post fasting) for dose volume calculation. Blood samples were collected from all rats prior to dosing with vehicle (0.5% methylcellulose) or test compound via oral gavage (5 mL/kg). Sixty minutes later rats were bled and immediately dose with an IP dose of glucose (2 g/kg). The rats were re-bled at 15, 30, 60 and 120 minutes post-glucose load. Blood samples (˜250 μL/time point) were collected into EDTA tubes with aprotinin/DPPIVi (0.6 TIU/20 μL per mL whole blood), for the determination of plasma glucose, insulin, and total amide GLP-1 concentrations. Blood tubes were inverted several times immediately following collection and placed on ice, then spun at 14,000 rpm in a refrigerated centrifuge for 5 minutes. Plasma samples were analyzed for glucose levels using a Roche 912 clinical chemistry analyzer and plasma insulin concentrations were determined using the Alpco Ultra-Sensitive Insulin Rat Elisa.

The results are presented as mean +/−SEM (standard error of the mean) unless otherwise stated. Statistical evaluation of the data is carried out using one-way analysis of variance (ANOVA) with appropriate post-hoc analysis between vehicle and treatment group. Differences compared to vehicle with a P<0.05 were considered statistically significant using Dunnett's T-test.

TABLE 2
Effect of Example 17 during IPGTT

| | | | Total |
|---|---|---|---|
| | Glucose | | Amide GLP-1 |
| | 0-120 min | Insulin 0-60 min | 0-120 min AUC |
| Dose | AUC (percent | AUC (percent | (percent vehicle |
| (Example 17) | vehicle response) | vehicle response) | response) |
| 1 mg/kg | 88 | 126 | 235 |
| 10 mg/kg | ++ | ++ | 237 |
| | 69 | 162 | |

++ p > 0.01 compared to vehicle

IPGTT studies were performed with Example 3, either prepared as described above (dosed as a suspension in 0.5% methylcellulose) or prepared as an amorphous dispersion (25% active) with hydroxyproylmethylcellulose-acetate succinate (dosed as a suspension in 0.5% methylcellulose/0.1%/0 polysorbate 80). Table 2 shows group mean values, with results expressed as percent of the vehicle response. Statistical significance is based on a comparison to the vehicle group.

TABLE 3
Effect of Example 3 during IPGTT experiment

| | Glucose 0-120 min | Insulin AUC |
|---|---|---|
| Example 3 Dose | AUC (percent | (0-60 min) (percent |
| and Formulation | vehicle response) | vehicle response) |
| 20 mg/kg in 0.5% | ++ | 104 |
| methylcellulose* | 85 | |
| 20 mg/kg SDD | ++ | 118 |
| | 89 | |

++ p > 0.01 compared to vehicle
*IP glucose administered 30 minutes post dose

### Preparation of Starting Materials

Preparation 1: Scheme A illustrates the preparation of syn and anti Isopropyl-9-hydroxy-3-oxa-7-azabicyclo[3.3.1]nonane-7-carboxylate. The experimental details are described in detail below.

### Preparation 1: Isopropyl-9-hydroxy-3-oxa-7-azabicyclo[3.3.1]nonane-7-carboxylate (mixture of syn- and anti-isomers)

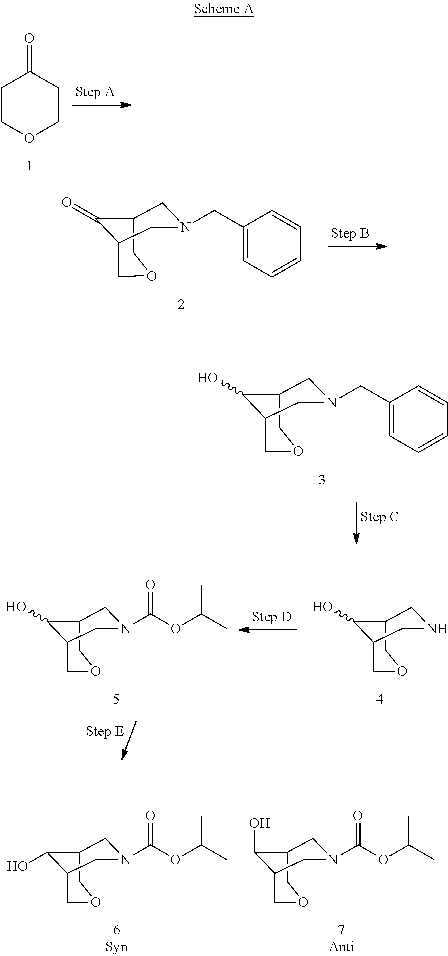

### Step A of Scheme A. Synthesis of 7-benzyl-3-oxa-7-azabicyclo[3.3.1]nonan-9-one-hydrochloride salt (2)

A solution of tetrahydro-4H-pyran-4-one 1 (60.0 g, 0.60 mol), benzylamine (63.4 g, 0.60 mol) and glacial acetic acid (35.9 g, 0.60 mol) in dry methanol (1.2 L) was added to a stirred suspension of paraformaldehyde (39.6 g, 1.3 mol) in dry methanol (1.2 L) over a period of 75 minutes at 65 degrees Celsius. A second portion of paraformaldehyde (39.6 g, 1.3 mol) was added, and the mixture was stirred for 1 hour at 65 degrees Celsius. The reaction was quenched with water (1.2 L) and 1 M aqueous potassium hydroxide solution (600 mL). The mixture was extracted with ethyl acetate (3 L×3). The combined organic layers were dried over sodium sulfate, filtered, and the filtrate was concentrated to dryness in vacuo. The residue was purified by column chromatography (petroleum ether/ethyl acetate=20:1˜2:1) to afford a brown oil. The residue was diluted with 6 M anhydrous hydrochloric acid in 1,4-dioxane (500 mL), and the mixture was stirred for 30 minutes. The solvent was removed in vacuo, and acetone (500 mL) was added. The resulting mixture was sonicated for 30 minutes causing a white precipitate to form. The mixture was filtered, and the solid was washed with acetone and then dried under vacuum to afford the desired product as a white solid (21 g, 13%): ¹H NMR (400 MHz, deuterium oxide) delta 7.43-7.42 (m, 5H), 4.66 (s, 2H), 3.95-3.90 (m, 4H), 3.54-3.47 (m, 4H); 1.96 (bs, 2H); LCMS (ES+): 232.0 (M+1).

### Step B of Scheme A. Synthesis of 7-benzyl-3-oxa-7-azabicyclo[3.3.1]nonan-9-ol (mixture of syn and anti-isomers) (3)

7-benzyl-3-oxa-7-azabicyclo[3.3.1]nonan-9-one hydrochloride salt (4.40 g, 16.9 mmol) was suspended in ethanol (40 mL) and anhydrous tetrahydrofuran (40 mL). The mixture was cooled with an ice bath, and sodium borohydride (1.5 g, 37.3 mmol) was added in one portion. The mixture was allowed to warm slowly over 4 hours to room temperature. The reaction was then concentrated in vacuo to remove most of the ethanol and tetrahydrofuran. The mixture was partitioned between methyl tert-butyl ether and aqueous 1.0 M sodium hydroxide solution. The solution was stirred for 30 minutes followed by separation of the two layers. The aqueous layer was extracted with methyl tert-butyl ether. The organic extracts were combined, washed with brine, and dried over sodium sulfate. The mixture was filtered and the filtrate was concentrated in vacuo to give a clear oil, which partially solidified on standing to an oily white solid (3.71 g, 94%). This mixture of syn and anti-7-benzyl-3-oxa-7-azabicyclo[3.3.1]nonan-9-ol isomers was used in the next step without further purification. LCMS (ES+): 234.1 (M+1).

An alternative procedure was performed as follows:

To a 20 L reactor equipped with a reflux condenser was added methanol (8.00 L; 6.33 kg), benzylamine (4.00 moles; 428.12 g), tetrahydro-4H-pyran-4-one (400 g, 4.00 moles), and acetic acid (4.00 moles; 239.93 g). The jacket temperature was maintained at 15-25 degrees Celsius during the addition. The reaction mixture was heated to reflux (66 degrees Celsius).

Aqueous formaldehyde (7.99 moles; 600.42 mL; 648.46 g) was combined with methanol (2 L). The resulting solution was added over 1 hour to the reaction while keeping the reaction at reflux. The reaction was heated for 10 minutes at reflux after the completion of formaldehyde addition, and cooled to 10-20 degrees Celsius. Sodium bicarbonate (4.00 moles; 335.63 g) was added. The reaction was cooled to 10 degrees Celsius, and sodium borohydride (4.20 moles; 158.71 g) was added portion-wise (sodium borohydride tablets were used, ˜1 g each tablet). After the sodium borohydride addition was complete, the reaction was stirred at 15-25 degrees Celsius for 40 min. Diatomaceous earth (400 g) was added to the reaction mixture, followed by water (2 L) and 1 N sodium hydroxide solution (4.00 L). The reaction mixture was stirred at 15-25 degrees Celsius for 1 hour, and filtered. The filter cake was rinsed with methanol/water (1:1 mixture, 800 mL). The filtrate was concentrated at 40-45 degrees Celsius under vacuum to remove most of the methanol. The resulting aqueous mixture was extracted with 2-methyl tetrahydrofuran (1×6.00 L). The 2-methyl tetrahydrofuran layer was washed with brine (2.00 L; 2.38 kg), concentrated under partial vacuum with a pot temperature of 40-45 degrees Celsius to give an oil, which was collected in a 5 L container (Naljug). The reactor was rinsed with 1 L of acetonitrile, and the rinse was combined with the crude oil product. After 12 hours standing at 10-15 degrees Celsius, crystallization occurred in the Naljug. Filtration of the mixture gave the syn-diastereomer (193 g, 98% de). The filtrate was purified by silica gel chromatography (mobil phase: toluene/heptane/diethylamine 70/30/5, isocratic), followed by another chromatography using ChiralPak AD (mobile phase: isopropanol/heptane/diethylamine 5/95/0.2) to give additional crop of syn-diastereomer (86.3 g) and anti-diastereomer (145 g).

Alternative enzymatic reduction procedures were also performed as follows:

### Enzymatic Procedure A

A reaction vial was charged with 75 microliters of a solution of niciotinamide adenine dinucleotide phosphate (NADH) (53 mg/mL, in 0.1 M potassium phosphate buffer, pH 7), 20 microliters of a solution of Codexis KRED-NADH 101 (Codexis, Inc., 200 Penobscot Drive, Redwood City, Calif. 94063) (50 mg/mL, in 0.1 M potassium phosphate buffer, pH 7) and 5 microliters of a solution of 7-benzyl-3-oxa-7-azabicyclo[3.3.1]nonan-9-one (200 mg/mL, in DMSO). The resulting mixture was stirred at 30 degrees Celsius for 20 hours. The reaction was diluted with ethyl acetate (900 microliters), mixed and centrifuged. The organic layer (600 microliters) was collected, evaporated to dryness and re-suspended in methanol (600 microliters) for analysis by super critical fluid chromatography (SFC). SFC analysis showed only formation of anti-7-benzyl-3-oxa-7-azabicyclo[3.3.1]nonan-9-ol isomer in 97% yield conversion. No evidence of the syn isomer was found.

### Enzymatic Procedure B

A reaction vial was charged with 75 microliters of a solution of NADH (53 mg/mL, in 0.1 M potassium phosphate buffer, pH 7), 20 microliters of a solution of DAICEL-E002 (Daicel Chemical Industries, Ltd., CPI Company, JR Shinagawa East Bldg. 2-18-1,Konan, Minato-ku Tokyo 108-8230, Japan) (50 mg/mL, in 0.1 M potassium phosphate buffer, pH 7) and 5 microliters of a solution of 7-benzyl-3-oxa-7-azabicyclo[3.3.1]nonan-9-one (200 mg/mL, in DMSO). The resulting mixture was stirred at 30 degrees Celsius for 20 hours. The reaction was diluted with ethyl acetate (900 microliters), mixed and centrifuged. The organic layer (600 microliters) was collected, evaporated to dryness and re-suspended in methanol (600 microliters) for analysis for SFC. SFC analysis showed only formation of syn-7-benzyl-3-oxa-7-azabicyclo[3.3.1]nonan-9-ol isomer in 99% yield conversion. No evidence of the anti isomer was found.

### Step C of Scheme A. Synthesis of 3-oxa-7-azabicyclo[3.3.1]nonan-9-ol (mixture of syn and anti-isomers) (4)

The starting mixture of syn and anti-7-benzyl-3-oxa-7-azabicyclo[3.3.1]nonan-9-ol isomers (3.71 g, 15.9 mmol) was dissolved in ethanol (120 mL), and Pd(OH)₂ (450 mg) was added. The mixture was shaken for 2.5 hours under 50 psi of hydrogen in a Parr shaker. The mixture was filtered through diatomaceous earth, and the collected solid was washed three times with methanol. The filtrate was concentrated in vacuo to give an oily solid. This oily solid was dissolved in ethyl acetate and heptane was added. The solution was concentrated in vacuo to give a mixture of syn and anti-isomers of 3-oxa-7-azabicyclo[3.3.1]nonan-9-ol as a white solid (2.08 g, 91%). This material was used in the next step without further purification. LCMS (ES+): 144.1 (M+1).

### Step D of Scheme A. Synthesis of isopropyl 9-hydroxy-3-oxa-7-azabicyclo[3.3.1]nonane-7-carboxylate (mixture of syn and anti-isomers) (5):

To a dichloromethane (15 mL) solution of the mixture of syn and anti-isomers of 3-oxa-7-azabicyclo[3.3.1]nonan-9-ol (2.08 g, 14.5 mmol) and N,N-diisopropylethylamine (2.80 mL, 16.0 mmol) at 0 degrees Celsius was added isopropyl chloroformate (14.2 mL, 14.2 mmol, 1.0 M in toluene) drop-wise. The reaction mixture was allowed to warm to room temperature over 14 hours. The reaction was then diluted with aqueous 1 M hydrochloric acid (50 mL), and the aqueous layer separated. The organic layer was washed sequentially with water (50 mL) and brine (50 mL) and then dried over sodium sulfate. The mixture was filtered, and the filtrate was concentrated in vacuo to give a colorless oil. This oil was dissolved in ethyl acetate; heptane was added and the mixture was concentrated. The resulting oil was dried under vacuum to give the mixture of syn and anti-isomers of isopropyl 9-hydroxy-3-oxa-7-azabicyclo[3.3.1]nonane-7-carboxylate as a clear oil (2.74 g, 82%). LCMS (ES+): 230.1 (M+1).

### Step E. Separation of the syn and anti-isomers of isopropyl-9-hydroxy-3-oxa-7-azabicyclo[3.3.1]nonane-7-carboxylate:

A mixture of syn and anti isomers of isopropyl 9-hydroxy-3-oxa-7-azabicyclo[3.3.1]nonane-7-carboxylate (5.04 g, 35.1 mmol) was separated via preparatory high pressure liquid chromatography utilizing a Chiralpak AD-H column (21×250 mm) with mobile phase of 85:15 carbon dioxide and methanol respectively at a flow rate of 65 mL/minute. The wavelength for monitoring the separation was 210 nm. The analytical purity of each isomer was determined using analytical high pressure chromatography using a Chiralpak AD-H (4.6 mm×25 cm) column with a mobile phase of 85:15 carbon dioxide and methanol respectively at a flow rate of 2.5 mL/minute. The wavelength for monitoring the peaks was 210 nm. The following two isomers were obtained:

Isopropyl-9-syn-hydroxy-3-oxa-7-azabicyclo[3.3.1]nonane-7-carboxylate (6) (1.34 g): clear oil which solidified on standing, Retention time (Rₜ)=2.3 minutes, ¹H NMR (400 MHz, deutero-DMSO): delta 5.12 (d, 1H, J=2.8 Hz), 4.76-4.71 (m, 1H), 4.20 (d, 1H, J=13 Hz), 4.16 (d, 1H, J=13 Hz), 3.96-3.92 (m, 2H), 3.79 (d, 1H, J=3 Hz), 3.55 (s, 1H), 3.52 (s, 1H), 3.08 (d, 1H, J=13 Hz), 2.98 (d, 1H, J=13 Hz), 1.47 (m, 2H) 1.16 (d, 3H, J=3 Hz), 1.15 (d, 3H, J=3 Hz); LCMS (ES+): 230.2 (M+1).

Isopropyl-9-anti-hydroxy-3-oxa-7-azabicyclo[3.3.1]nonane-7-carboxylate (7) (1.70 g): amber oil, Rₜ=3.08 minutes, ¹H NMR (400 MHz, deutero-DMSO): delta 5.11 (d, 1H, J=2.8 Hz), 4.74-4.67 (m, 1H), 3.89 (d, 1H, J=13 Hz), 3.84-3.78 (m, 2H, J=11 Hz), 3.80 (d, 1H, J=6 Hz), 3.78 (d, 1H, J=3 Hz), 3.52-3.47 (m, 2H), 3.35-3.30 (m, 1H), 3.24-3.20 (m, 1H), 1.53 (s, 1H), 1.51 (s, 1H), 1.13 (d, 3H, J=1 Hz), 1.16 (d, 3H, J=1 Hz); LCMS (ES+): 230.2 (M+1)

Alternatively, steps A and B from reaction Scheme A, above, can be combined as described below for the synthesis of 7-benzyl-3-oxa-7-azabicyclo[3.3.1]nonan-9-ol (mixture of syn and anti-isomers):

Benzylamine (21.35 g, 199.27 mmol), tetrahydro-4H-pyran-4-one (1) (19.95 g, 199.27 mmol) and acetic acid (11.97 g, 199.27 mmol) were dissolved in methanol (400 mL). The mixture was heated at reflux. A solution of aqueous formaldehyde (37%, 32.34 g, 398.53 mmol) and methanol (100 mL) was added to the reaction mixture over a period of 60 minutes, keeping the reaction at reflux. The reaction was cooled to room temperature. Sodium bicarbonate (16.74 g, 199.27 mmol) was then added portion-wise. Subsequently, sodium borohydride (7.92 g 209.23 mmol) was added portion-wise, maintaining the reaction temperature at 25 degrees Celsius or lower. The mixture was stirred at ambient temperature for 30 minutes. Diatomaceous earth (20 g) was added, followed by water (100 mL) and aqueous 1N sodium hydroxide solution (100 mL). After it was stirred for 1 hour, the mixture was filtered and the filter cake was rinsed sequentially with methanol and water (20 mL each). The filtrate was concentrated in vacuo to remove most of the methanol. The resulting aqueous mixture was extracted with 2-methyltetrahydrofuran (300 mL). The organic phase was washed with brine solution (100 mL), dried over anhydrous magnesium sulfate, and concentrated in vacuo to provide a mixture of syn and anti-7-benzyl-3-oxa-7-azabicyclo[3.3.1]nonan-9-ol isomers as an oil that solidified upon standing at room temperature (22.0 g, 47.3%).

### Preparation 2: tert-Butyl 9-hydroxy-3-oxa-7-azabicyclo[3.3.1]nonane-7-carboxylate (mixture of syn- and anti-isomers)

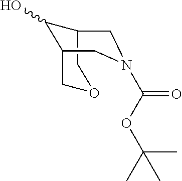

To a 0 degrees Celsius solution of 3-oxa-7-azabicyclo[3.3.1]nonan-9-ol (mixture of syn- and anti-isomers) (3.78 g, 26.4 mmol) in water (30 mL) and tetrahydrofuran (30 mL) was added drop-wise a solution of di-tert-butyl dicarbonate (5.76 g, 26.4 mmol) in tetrahydrofuran (20 mL). The solution was allowed to stir for approximately 15 hours while warming gradually to room temperature. The reaction was diluted with dichloromethane and water. The layers were separated, and the aqueous layer was extracted with dichloromethane. The organic layers were combined and dried over sodium sulphate. The mixture was filtered, and the filtrate concentrated under reduced pressure to reveal the title compound as a clear oil (6.55 g) which was used without further purification.

### Preparation 3: Separation of the syn and anti-isomers of tert-butyl 9-hydroxy-3-oxa-7-azabicyclo[3.3.1]nonane-7-carboxylate

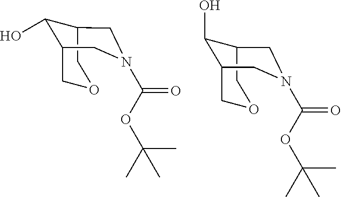

A mixture of syn- and anti-isomers of tert-butyl 9-hydroxy-3-oxa-7-azabicyclo[3.3.1]nonane-7-carboxylate (5.04 g, 35.1 mmol) was separated via preparatory high pressure liquid chromatography utilizing a Chiralpak AD-H column (21×250 mm) with mobile phase of 85:15 carbon dioxide and methanol respectively at a flow rate of 65 mL/minute. The wavelength for monitoring the separation was 210 nm.

The analytical purity of each isomer was determined using analytical high pressure chromatography using a Chiralpak AD-H (4.6 mm×25 cm) column with a mobile phase of 85:15 carbon dioxide and methanol respectively at a flow rate of 2.5 mL/minute. The wavelength for monitoring the peaks was 210 nm. The following two isomers were obtained:

tert-Butyl 9-anti-hydroxy-3-oxa-7-azabicyclo[3.3.1]nonane-7-carboxylate: (1.30 g, 100% de); clear oil which solidified to a white solid on standing, Retention time (Rₜ)=3.15 minutes; ¹H NMR (400 MHz, deuterochloroform) delta 1.44 (s, 9H), 1.66 (d, J=16.79 Hz, 2H), 1.84 (d, J=2.93 Hz, 1H), 3.30-3.52 (m, 2H), 3.64 (t, J=11.03 Hz, 2 H), 3.93-4.21 (m, 5H).

tert-Butyl 9-syn-hydroxy-3-oxa-7-azabicyclo[3.3.1]nonane-7-carboxylate: (1.64 g, 89% de); clear oil which solidified to a white solid on standing, Rₜ=3.55 minutes; ¹H NMR (400 MHz, deuterochloroform) delta 1.47 (s, 9H), 1.64 (d, J=13.47 Hz, 2H), 2.12 (d, J=3.32 Hz, 1H), 2.92-3.22 (m, 2H), 3.71-3.83 (m, 2H), 3.99 (d, J=3.32 Hz, 1H), 4.09-4.19 (m, 2H), 4.32 (d, J=13.66 Hz, 1H), 4.48 (d, J=13.66 Hz, 1H).

### Preparation 4: 1-(6-chloropyrimidin-4-yl)-5-(methylthio)indoline

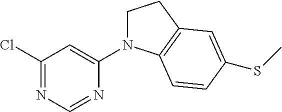

To a stirred solution of 4,6-dichloropyrimidine (8.60 g, 57.7 mmol) in n-propanol (110 mL) at 107 degrees Celsius was added rapidly a solution of 5-(methylthio)indoline (WO199501976) (8.81 g) in n-propanol (60 mL). The reaction mixture was heated at 107 degrees Celsius for 45 minutes. After it was cooled to room temperature, the mixture was diluted with methyl tert-butyl ether (125 mL). The resulting solid was collected by filtration, and the filter cake was washed with methyl tert-butyl ether. The filtrate was discarded, and the filter cake was partitioned between saturated aqueous sodium bicarbonate solution (200 mL), and chloroform (500 mL). The biphasic mixture was heated in a water bath at 40 degrees Celsius to dissolve all remaining solids. The organic solution was then separated and dried over magnesium sulfate. The mixture was filtered, and the filtrate was concentrated under reduced pressure to give 1-(6-chloropyrimidin-4-yl)-5-(methylthio)indoline as a yellow solid (14.8 g, 84%); ¹H NMR (400 MHz, deuterochloroform) delta 2.49 (s, 3H) 3.27 (t, J=8.5 Hz,2H), 4.02 (t, J=8.5 Hz, 2H), 6.59 (s, 1H), 7.17-7.20 (m, 2H), 8.35 (d, J=8.7 Hz, 1H), 8.58 (s, 1H); LCMS (ES+): 278.4 (M+1).

### Preparation 5: 1-(6-chloropyrimidin-4-yl)-5-(methylsulfonyl)indoline

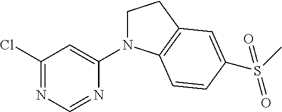

To a stirred solution of 1-(6-chloropyrimidin-4-yl)-5-(methylthio)indoline (12.4 g, 44.5 mmol) in chloroform (300 mL) at 50 degrees Celsius was added a dry solution of meta-chloroperoxybenzoic acid (27.4 g, 111 mmol) in chloroform (150 mL) (prepared by dissolution in warm chloroform and discarding the separated aqueous layer). After 1 hour, the reaction mixture was quenched with dimethylsulfide (1.7 mL), stirred for 10 minutes and then poured into a solution of 10% aqueous sodium carbonate (150 mL). The aqueous layer was separated, and the organic layer was washed with 10% aqueous sodium carbonate (100 mL) and dried over magnesium sulfate. The mixture was filtered, and the filtrate was concentrated under reduced pressure. The resulting residue was dissolved in hot acetonitrile. Upon cooling, 1-(6-chloropyrimidin-4-yl)-5-(methylsulfonyl)indoline precipitated as a white solid (13.8 g, 85%). ¹H NMR (400 MHz, deuterochloroform) delta 3.05 (s, 3H), 3.36 (t, J=8.7 Hz, 2H), 4.12 (t, J=8.7 Hz, 2H), 6.68 (s, 1H), 7.74-7.85 (m, 1H), 7.81 (d, J=8.4 Hz 1H), 8.66 (s, 1H), 8.63 (d, J=8.4 Hz, 1H); LCMS (ES+) 310.4 (M+1).

### Preparation 6: 4-chloro-6-[5-(methylthio)-2,3-dihydro-1H-indol-1-yl]pyrimidine-5-carbonitrile

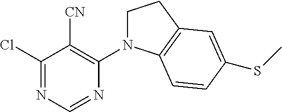

5-(Methylthio)indoline (50 mg, 0.30 mmol) in acetonitrile (1 mL) was heated at 80 degrees Celsius for 2 minutes and then cooled to room temperature. 4,6-Dichloropyrimidine-5-carbonitrile (WO2006118749) (53 mg, 0.30 mmol) and diisopropylethylamine (0.10 mL, 0.45 mmol) were added to the reaction mixture which was then stirred for 3 hours. The reaction mixture was concentrated under reduced pressure, and the residue was triturated with heptane. The mixture was filtered to give 4-chloro-6-[5-(methylthio)-2,3-dihydro-1H-indol-1-yl]pyrimidine-5-carbonitrile as a white solid (40 mg, 43%). ¹H NMR (400 MHz, deutero-DMSO) delta 8.63 (s, 1H), 8.09 (d, 1 H, J=8.8 Hz,), 7.24 (s, 1H), 7.12 (d, 1H, J=8.4 Hz), 4.49 (t, 2H, J=8.2 Hz), 3.21 (t, 2H, J=8.0 Hz), 2.45 (s, 3H).

### Preparation 7: Isopropyl 4-[6-chloro-5-methoxypyrimidin-4-yl)oxy]piperidine-1-carboxylate

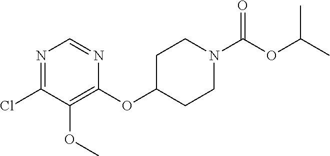

To a solution of 4,6-dichloro-5-methoxypyrimidine (240 mg, 1.34 mmol) and isopropyl 4-hydroxypiperidine-1-carboxylate (326 mg, 1.74 mmol) in anhydrous 1,4-dioxane (3 mL) at 100° C., was added a 1 M solution of sodium bis(trimethylsilyl)amide in tetrahydrofuran (1.34 mL, 1.34 mmol, 1.0 M). The reaction mixture was heated for 10 hours and then allowed to cool to room temperature. The reaction was then quenched with water (3 mL) and diluted with ethyl acetate (20 mL). The solution was then washed with sequentially with saturated aqueous sodium bicarbonate solution (10 mL) and brine (10 mL) followed by drying over sodium sulfate. The mixture was filtered, and the filtrate was concentrated under reduced pressure. The crude residue was purified by column chromatography (0-100% ethyl acetate in heptane) to give isopropyl 4-[(6-chloro-5-methoxypyrimidin-4-yl)oxy]piperidine-1-carboxylate as oil (260 mg, 59%). ¹H NMR (400 MHz, deuterochloroform) delta 8.25 (1H, s) 5.29-5.44 (1H, m) 4.84-5.01 (1H, m) 3.90 (3H, s) 3.72-3.82 (2H, m) 3.31-3.47 (2H, m) 1.93-2.10 (2H, m) 1.72-1.89 (2H, m) 1.25 (6H, d, J=6.24 Hz); LCMS (ES+): 329.0 (M+1).

### Preparation 8: Isopropyl 4-[6-chloro-5-methylpyrimidin-4-yl)oxy]piperidine-1-carboxylate

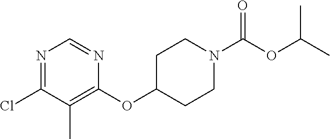

To a solution of 4,6-dichloro-5-methyl-pyrimidine (3.0 g, 18.4 mmol) and isopropyl 4-hydroxypiperidine-1-carboxylate (3.79 g, 20.2 mmol) in anhydrous tetrahydrofuran (100 mL) at 0 degrees Celsius was added a 1 M solution of potassium tert-butoxide in tetrahydrofuran (3.1 g, 27.6 mmol). The reaction was allowed to warm to room temperature while stirring for 18 hours. The reaction was then quenched with water and extracted with ethyl acetate four times. The organic extracts were combined and dried over sodium sulfate. The mixture was filtered, and the filtrate was concentrated under reduced pressure. The resulting material was purified by column chromatography (0-50% ethyl acetate in heptane) to give isopropyl 4-[(6-chloro-5-methylpyrimidin-4-yl)oxy]piperidine-1-carboxylate as a white solid (4.4 g, 76%). ¹H NMR (400 MHz, deuterochloroform) delta 1.23 (d, J=6.4 Hz, 6H) 1.70-1.79 (m, 2H) 1.91-2.01 (m, 2H) 2.20 (s, 3H) 3.34-3.42 (m, 2H) 3.67-3.77 (m, 2H) 4.86-4.95 (m, 1H) 5.29-5.35 (m, 1H) 8.36 (s, 1H); LCMS (ES+): 314.2 (M+1).

### Preparation 9: Isopropyl 4-[6-chloro-pyrimidin-4-yl)oxy]piperidine-1-carboxylate

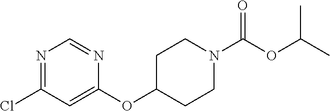

To a solution of isopropyl 4-hydroxypiperidine-1-carboxylate (660 mg, 3.52 mmol) and 4,6-dichloropyrimidine (500 mg, 3.36 mmol) in tetrahydrofuran (15 mL) was added a 1 M solution of potassium tert-butoxide in tetrahydrofuran (5.03 mL, 5.03 mmol) at 0 degrees Celsius. The reaction mixture was allowed to slowly warmed to room temperature overnight. After 18 hours, the reaction mixture was diluted with water and extracted three times with ethyl acetate. The combined organic layers were dried over sodium sulfate and then filtered, and the filtrate concentrated under reduced pressure. The crude residue was purified by column chromatography (0-50% ethyl acetate in heptane) to afford isopropyl 4-[(6-chloro-pyrimidin-4-yl)oxy]piperidine-1-carboxylate (700 mg, 69.6) as a colorless oil. ¹H NMR (400 MHz, deuterochloroform) delta 1.25 (d, J=6.25 Hz, 6H) 1.68-1.79 (m, 2H) 1.94-2.03 (m, 2H) 3.29-3.37 (m, 2 H) 3.75-3.83 (m, 2H) 4.88-4.97 (m, 1H) 5.28-5.36 (m, 1H) 6.75 (d, J=0.78 Hz, 1H) 8.55 (d, J=0.98 Hz, 1H). LCMS (ES+): 300.3 (M+1).

### Preparation 10:1-(6-Chloro-pyrimidin-4-yl)-2,3-dihydro-1H-indole-5-carboxylic acid methyl ester

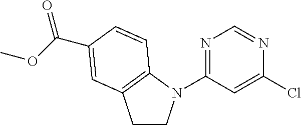

To a solution of 4,6-dichloropyrimidine (815 mg, 4.60 mmol) in n-propanol (12.0 mL) was added 2,3-dihydro-1H-indole-5-carboxylic acid methyl ester (Bioorg. Med. Chem. Lett. 2008, 18, 5684-8) (754 mg, 5.06 mmol). The resulting yellow solution was heated to reflux for 2 hours. After allowing the reaction to cool to room temperature, the reaction mixture was diluted with methyl tert-butyl ether and then filtered. The collected solids were partitioned between chloroform and saturated aqueous sodium bicarbonate solution. The separated organic phase was dried over magnesium sulfate, filtered, and the filtrate concentrated under reduced pressure to afford 1-(6-chloro-pyrimidin-4-yl)-2,3-dihydro-1H-indole-5-carboxylic acid methyl ester (929 mg, 69.7%) as an off-white solid. ¹H NMR (400 MHz, deuterochloroform) delta 3.32 (t, J=8.49 Hz, 2H) 3.91 (s, 3H) 4.08 (t, J=8.69 Hz, 2H) 6.67 (s, 1H) 7.90 (d, J=0.98 Hz, 1H) 7.96 (dd, J=8.49, 1.46 Hz, 1H) 8.46 (d, J=8.59 Hz, 1H) 8.65 (s, 1H).

### Preparation 11: 1-(6-Chloro-pyrimidin-4-yl)-2,3-dihydro-1H-indole-5-carboxylic acid

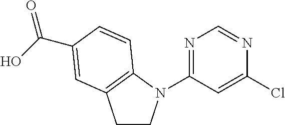

To a solution of 1-(6-chloro-pyrimidin-4-yl)-2,3-dihydro-1H-indole-5-carboxylic acid methyl ester (100 mg, 0.345 mmol) in a solution of tetrahydrofuran (3 mL) and water (1 mL) was added lithium hydroxide monohydrate (16.8 mg, 0.380 mmol). The reaction mixture was heated to 60 degrees Celsius. After 4 hours, the reaction mixture was allowed to cool to room temperature causing a precipitate to form in the solution. The mixture was filtered, and the collected solid dried under reduced pressure to afford 1-(6-chloro-pyrimidin-4-yl)-2,3-dihydro-1H-indole-5-carboxylic acid (67 mg, 70%) as a white solid. ¹H NMR (400 MHz, deutero-DMSO) delta 3.24 (t, J=8.78 Hz, 2H) 4.10 (t, J=8.69 Hz, 2H) 7.02 (s, 1H) 7.77 (d, J=1.17 Hz, 1H) 7.81 (dd, J=8.49, 1.85 Hz, 1H) 8.44 (d, J=8.59 Hz, 1H) 8.62 (s, 1H) 12.60 (broad. s., 1H). LCMS (ES+): 276.5 m/z (M+1).

### Preparation 12: Methyl 1-(6-chloro-5-methylpyrimidin-4-yl)indoline-5-carboxylate

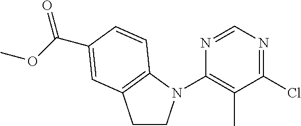

To a stirred solution of 4,6-dichloro-5-methylpyrimidine (101 mg, 0.62 mmol) in n-propanol (2.0 mL) was added 2,3-dihydro-1H-indole-5-carboxylic acid methyl ester (Bioorg. Med. Chem. Lett. 2008, 18, 5684-8) (100 mg, 0.56 mmol). The resulting yellow solution was heated at reflux (100 degrees Celsius). After 4 hours at reflux the reaction mixture was cooled to room temperature and was concentrated in vacuo. The reside was purified by flash chromatography, eluting with a gradient mixture of 10-40% ethyl acetate to heptane to give methyl 1-(6-chloro-5-methylpyrimidin-4-yl)indoline-5-carboxylate as a white solid (60 mg).

### Preparation 13: tert-Butyl 5-[2-hydroxyethyl)thio]indoline-1-carboxylate

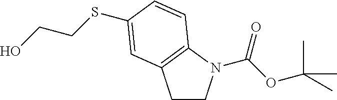

A solution of 5-bromo-2,3-dihydro-indole-1-carboxylic acid tert-butyl ester (933 mg, 3.13 mmol), diisopropylethylamine (1.1 mL, 6.26 mmol) in anhydrous 1,4-dioxane (20 mL) was purged with a stream of nitrogen for 10 minutes. 4,5-Bis (diphenylphosphino)-9,9-dimethylxanthene (201.4 mg, 0.34 mmol), tris(dibenzylideneacetone)dipalladium (148.4 mg, 0.162 mmol) and 2-mercaptoethanol (0.220 mL. 3.13 mmol) were then added sequentially, and the reaction mixture was heated at 110 degrees Celsius for 24 hours. The reaction mixture was cooled to room temperature and filtered through a pad of diatomaceous earth. The filtrate was then washed twice with water (50 mL). The combined organic layers were dried over magnesium sulfate, filtered, and the filtrate was concentrated under reduced pressure to give a crude yellow oil, which was purified by column chromatography to afford tert-butyl 5-[(2-hydroxyethyl)thio]indoline-1-carboxylate (898 mg, 97%) as a thick yellow oil. ¹H NMR (500 MHz, deuterochloroform) delta 1.57 (s, 9H) 3.02 (t, 2H) 3.08 (t, 2H) 3.69 (br. s., 2H) 3.96-4.03 (m, 2H) 7.25 (s, 1H) 7.26 (s, 1H) 7.28 (s, 1H) 7.79 (broad s, 1 H).

### Preparation 14: 2-(2,3-Dihydro-1H-indol-5-ylthio)ethanol

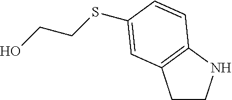

To a solution of tert-butyl 5-[(2-hydroxyethyl)thio]indoline-1-carboxylate (890 mg, 3.01 mmol) in 1,4-dioxane (8.0 mL) was added a 4 M solution of hydrochloric acid in 1,4-dioxane (2.0 mL). The reaction was stirred for 15 minutes followed by sequential heating to 50 degrees Celsius for 20 minutes and then 75 degrees Celsius for 30 minutes. The reaction was allowed to cool to room temperature, and the solid was filtered to afford 2-(2,3-dihydro-1H-indol-5-ylthio)ethanol (376 mg, 64%) as a light brownish, orange solid. ¹H NMR (400 MHz, deuteromethanol) delta 3.11 (t, 2H) 3.30-3.33 (m, 2H) 3.69 (t, 2H) 3.84 (t, 2H) 7.38-7.39 (m, 2H) 7.48-7.50 (m, 1H).

### Preparation 15: 5-[(2-{[tert-Butyl(dimethyl)silyl]oxy}ethyl)thio]indoline

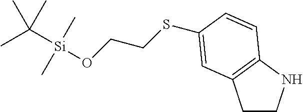

To a suspension of 2-(2,3-dihydro-1H-indol-5-ylthio)ethanol in dichloromethane (8 mL) was sequentially added triethylamine (0.6 mL, 4.0 mmol), 4-dimethylaminopyridine (21.8 mg, 0.18 mmol) and tert-butyldimethylsilyl chloride (244 mg,1.62 mmol. The reaction was stirred for 3 hours, and concentrated under reduced pressure. The residue was diluted with ethyl acetate (20 mL). The mixture was filtered, and filtrate concentrated to give a crude orange oil that was purified by column chromatography to give 5-[(2-{[tert-butyl(dimethyl)silyl]-oxy}ethyl)-thio]indoline (145 mg. 34%) as an oil. ¹H NMR (400 MHz, deuterochloroform) delta 0.01 (s, 6H) 0.85 (s, 9H) 2.83-2.90 (m, 2H) 2.99 (t, J=8.39 Hz, 2H) 3.55 (t, J=8.49 Hz, 2H) 3.67-3.75 (m, 2H) 6.52 (d, J=8.00 Hz, 1H) 7.10 (dd, J=8.00, 1.95 Hz, 1H) 7.19 (d, J=1.37 Hz, 1H).

### Preparation 16: 2,3-dihydro-1H-pyrrolo[3,2-b]pyridine

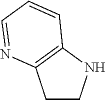

### Step A: tert-Butyl 1H-pyrrolo[3,2-b]pyridine-1-carboxylate

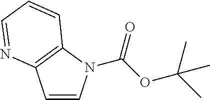

4-azaindole (50.05 g, 426 mmol) as a red solid was dissolved in tetrahydrofuran (380 mL) to give a deep red colored solution. The di-tert butylcarbonate (95.24 g, 430 mmol) was dissolved in tetrahydrofuran (50 mL) and was slowly added drop-wise by addition funnel over the time of 75 minutes to the solution of the azaindole. The flow rate was approximately 2 mL/min. The lengthy addition was used to regulate the carbon dioxide evolution. The addition caused the color of the reaction mixture to turn lighter and more orange in color. The mixture was stirred for 16 hours at room temperature before the reaction was concentrated to dryness under vacuum. The orange residue solidified to give a 93.84 g of a tan colored solid (MS ES+: 163.2 [M-tBu]). This material was used in the subsequent step without further purification.

### Step B: tert-Butyl 2,3-dihydro-1H-pyrrolo[3,2-b]pyridine-1-carboxylate

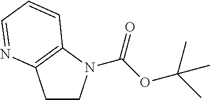

Palladium hydroxide (3.22 g,˜13 mol % palladium, Aldrich, 330094) wetted with minimal ethanol was added to a 500 mL Parr bottle under a nitrogen atmosphere. To this was added the crude tert-butyl 1H-pyrrolo[3,2-b]pyridine-1-carboxylate (10.0 g) as a solid. Ethanol (160 mL) was added and the mixture was shaken under a 20 psi hydrogen atmosphere. The mixture was heated at 60 degrees Celsius and the hydrogen pressure was increased to 50 psi. The hot mixture was shaken under a 50 psi atmosphere of hydrogen for 30 hours before the mixture was cooled to room temperature and filtered through a Pall GHP membrane (0.45 micrometer), rinsing with ethanol. The filtrate was concentrated under vacuum to give 9.95 g of tert-butyl 2,3-dihydro-1H-pyrrolo[3,2-b]pyridine-1-carboxylate as a yellow oil that was used in the subsequent step without purification. Step C: 2,3-Dihydro-1H-pyrrolo[3,2-b]pyridine

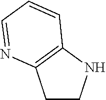

Hydrogen chloride (45.2 mL; 4 N in dioxane) was added to a stirred solution of tert-butyl 2,3-dihydro-1H-pyrrolo[3,2-b]pyridine-1-carboxylate (9.95 g) in 45 mL of methanol at room temperature. The mixture was heated to 60 degrees Celsius for 1 hour. The mixture was cooled to room temperature, diluted with diethyl ether and the solid precipitate was collected by filtration. Drying of the collected solids under vacuum gave the hydrochloride salt as a tan solid. 1.0 g of this salt was dissolved in 10 mL methanol. The mixture was cooled to 0 degrees Celsius and was added aqueous potassium hydroxide (0.97 mL, 11.8 M, 11.45 mmol, 2.2 eq). Once the base was added, the cold bath was removed and the reaction mixture was stirred at room temperature for 5 minutes. The mixture was concentrated under vacuum to near dryness before adding 20 mL dichloromethane. The mixture was dried over sodium sulfate, filtered through a coarse fritted glass funnel, and the solids were rinsed with dichloromethane. The filtrate was concentrated to dryness under vacuum to give 2,3-dihydro-1H-pyrrolo[3,2-b]pyridine as an orange oil that solidified upon standing to give 0.6 g, 96%. ¹H NMR (500 MHz, deuterochloroform) delta 3.16 (t, 2H) 3.65 (td, J=8.66, 1.71 Hz, 2H) 3.78 (br. s., 1H) 6.79-6.84 (m, 1H) 6.89 (dd, J=7.56, 5.37 Hz, 1H) 7.87 (dd, J=5.00, 1.10 Hz, 1H)

### Preparation 17: 1-(6-chloropyrimidin-4-yl)-2,3-dihydro-1H-pyrrolo[3,2-b]pyridine

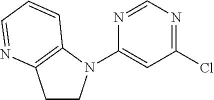

A mixture of 2,3-dihydro-1H-pyrrolo[3,2-b]pyridine (200 mg, 1.66 mmol) (J. Med. Chem., 1998, 41, 1598) and 1-propanol (6 mL) was heated at 110 degrees Celsius to form a solution. The solution was allowed to cool to room temperature, and 4,6-dichloropyrimidine (248 mg, 1.66 mmol) was added, and the reaction heated at 115 degrees Celsius for 3 hours and then allowed to cool to room temperature. The reaction was diluted with ethyl acetate and water, and the aqueous phase was extracted twice with ethyl acetate. The combined extracts were washed with sequentially with water and then brine followed by drying over sodium sulfate. The mixture was filtered, and the filtrate was concentrated under reduced pressure to a solid, which was purified by column chromatography (5% methanol/0.5% triethylamine in dichloromethane) to give 1-(6-chloropyrimidin-4-yl)-2,3-dihydro-1H-pyrrolo[3,2-b]pyridine (100 mg, 26%) as a foam. ¹H NMR (400 MHz, chloroform-d) delta 8.64 ppm (d, J=8.31 Hz, 1H) 8.60 ppm (s, 1H) 8.15 ppm (d, J=4.98 Hz, 1H) 7.15 ppm (d, J=8.31 Hz, 1H) 6.62 ppm (s, 1H) 4.04 ppm (t, 2H) 3.42 ppm (t, 2H). LCMS (ES+): 233 (M+1)

### Preparation 18: 1-(6-chloro-5-methylpyrimidin-4-yl)-2,3-dihydro-1H-pyrrolo[3,2-b]Pyridine

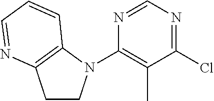

This compound was prepared from 2,3-dihydro-1H-pyrrolo[3,2-b]pyridine and 4,6-dichloro-5-methylpyrimidine using a procedure analogous to that in Preparation 17. 1-(6-chloro-5-methylpyrimidin-4-yl)-2,3-dihydro-1H-pyrrolo[3,2-b]pyridine (100 mg, 26%) was isolated as a tan solid. ¹H NMR (400 MHz, deuterochloroform) delta 8.49 (s, 1H), 8.09 (d, J=4.98 Hz, 1H), 7.21 (d, J=7.89 Hz, 1H), 7.05 (d, J=7.89 Hz, 1H), 4.20 (t, 2 H), 3.31 (t, 2H), 2.29 (s, 3H). LCMS (ES+): 247 (M+1).

An alternative procedure is as follows:

To a solution of 2,3-dihydro-1H-pyrrolo[3,2-b]pyridine (9.05 g, 75.3 mmol) and dichloropyrimidine (12.3 g, 75.3 mmol) in tert-butanol (90 mL) and toluene (90 mL) was added cesium carbonate (37.6 g). The mixture was degassed with a stream of nitrogen gas. Bis(triphenylphosphine)palladium(II) dichloride (1.59 g) was added and the mixture was again degassed with nitrogen for several minutes. The resulting mixture was heated at reflux (115 degrees Celsius) for 18 hours. The mixture was cooled to room temperature, diluted with ethyl acetate and the mixture was filtered through diatomaceous earth. The filtrate was concentrated in vacuo to an oil. This oil was dissolved in 2-methyl tetrahydrofuran (400 mL) and 300 mL of 1 N aqueous hydrochloric acid was added. The aqueous layer was separated and extracted twice with 2-methyl tetrahydrofuran. The combined organic extracts were washed with water. The combined aqueous layers were cooled in an ice bath and triethylamine was added slowly until the pH was adjusted to 8 to 9. A precipitate formed and these solids were collected by filtration. The aqueous filtrate was extracted twice with 2-methyl tetrahydrofuran. These combined organic extracts were washed with brine, dried over sodium sulfate, filtered and the filtrate was concentrated in vacuo. The residue was combined with the previously collected solids, dissolved in 2-methyl tetrahydrofuran and concentrated in vacuo. The reside was purified by flash chromatography using 330 g of silica gel, eluting with a gradient mixture of heptane and ethyl acetate (50 to 100% over 30 min and then 100% ethyl acetate for 30 min) to give 1-(6-chloro-5-methylpyrimidin-4-yl)-2,3-dihydro-1H-pyrrolo[3,2-b]pyridine as an off-white solid (25.5 g). ¹H NMR (400 MHz, deuterochloroform) delta 2.27 (s, 3H) 3.29 (t, J=8.39 Hz, 2H) 4.18 (t, J=8.39 Hz, 2H) 7.03 (dd, J=8.10, 4.98 Hz, 1H) 7.20 (dd, J=8.10, 1.27 Hz, 1H) 8.07 (dd, J=5.08, 1.37 Hz, 1H) 8.47 (s, 1H)

### Preparation 19: 2,3-Dihydro-1H-pyrrolo[2,3-b]pyridine

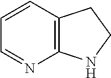

7-Azaindole (3.01 g, 25.5 mmol), p-toluenesulfonic acid monohydrate (4.86 g, 25.5 mmol), and formic acid (14 mL of 95% solution) were dissolved in 30 mL of 1-propanol. The reaction mixture was placed in a preheated 125° C. bath with stirring. Raney nickel (6 mL of 2800 active catalyst suspension, Aldrich) was added, and the mixture continued heating at 125 degrees Celsius for 1 hour. The mixture was then allowed to cool to 25 degrees Celsius and was filtered through diatomaceous earth. The solids were washed with 1-propanol to give a clear, light green filtrate. Disodium ethylenediaminetetraacetic acid (EDTA) dihydrate (2.5 g) was dissolved in the filtrate followed by the addition of aqueous 6 M sodium hydroxide solution (50 mL). The mixture was heated at reflux for 20 minutes, cooled to room temperature, and the 1-propanol phase was separated and concentrated under reduced pressure. The remaining basic, aqueous phase was extracted with 50 mL methyl tert-butyl ether and separated. The concentrated residue from the 1-propanol phase was taken up in 50 mL of methyl tert-butyl ether and 10 mL of water. The layers were separated, and the two methyl tert-butyl ether phases were combined and washed with two 10 mL portions of water, one 10 mL portion of brine, and dried over magnesium sulfate. The mixture was filtered, and the filtrate was concentrated under reduced pressure to give a white solid. This was recrystallized from 10 mL of hexanes to give 2,3-dihydro-1H-pyrrolo[2,3-b]pyridine (1.87 g, 61%) as a pale, tan solid. ¹H NMR (400 MHz, deuterochloroform) delta 3.06 (t, J=8.4 Hz, 2H), 3.61 (t, J=8.3 Hz, 2H), 4.51 (broad s., 1H), 6.50 (dd, J=7.0, 5.3 Hz, 1H), 7.24 (dd, J=7.0, 1.4 Hz, 1H), 7.82 (d, J=5.3 Hz, 1H). LCMS (ES): 121 (M+1).

### Preparation 20: 5-(Methylthio)-2,3-dihydro-1H-pyrrolo[2,3-b]pyridine

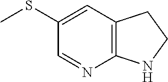

This compound was synthesized in a similar manner to 5-(methylthio)indoline (WO199501976) utilizing 2,3-dihydro-1H-pyrrolo[2,3-b]pyridine as a starting material. ¹H NMR (500 MHz, deuterochloroform) delta 2.37 (s, 3H), 3.06 (t, J=8.4 Hz, 2H), 3.64 (td, J=8.4, 1.3 Hz, 2H), 4.60 (br. s., 1H), 7.33 (s, 1H), 7.90 (s, 1H). LCMS (ES+): 167 (M+1).

### Preparation 21: 1-(6-chloropyrimidin-4-yl)-5-(methylthio)-2,3-dihydro-1H-pyrrolo[2,3-b]pyridine

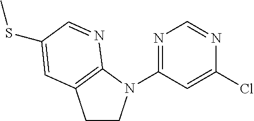

5-(Methylthio)-2,3-dihydro-1H-pyrrolo[2,3-b]pyridine (356 mg, 2.1 mmol) and 4,6-dichloropyrimidine (354 mg, 2.4 mmol) were dissolved in 4 mL of anhydrous 1,4-dioxane, and the mixture was placed in a preheated 100° C. oil bath. A 1 M solution of sodium bis(trimethylsilyl)amide in tetrahydrofuran (2.1 mL) was added in rapidly drop-wise causing a dark mixture to form at once. The mixture was heated for 30 minutes, allowed to cool to room temperature, and concentrated under reduced pressure. The residue was partitioned between ethyl acetate and water. The organic layer was separated, washed sequentially with water twice and then brine followed by drying over magnesium sulfate. The mixture was filtered, and the filtrate concentrated under reduced pressure give a red solid which was purified by column chromatography (heptane-ethyl acetate gradient) to give 1-(6-chloropyrimidin-4-yl)-5-(methylthio)-2,3-dihydro-1H-pyrrolo[2,3-b]pyridine as an off-white solid (363 mg, 61%). ¹H NMR (400 MHz, deuterochlorofom) delta 2.49 (s, 3H), 3.18 (t, J=8.6 Hz, 2H), 4.30-4.36 (m, 2H), 7.49 (d, J=2.1 Hz, 1H), 8.15 (d, J=2.1 Hz, 1H), 8.61 (d, J=0.8 Hz, 1H), 8.80 (d, J=1.0 Hz, 1H). LCMS (ES+): 279 (M+1).

### Preparation 22: 1-(6-chloropyrimidin-4-yl)-5-(methylsulfonyl)-2,3-dihydro-1H-pyrrolo[2,3-b]pyridine

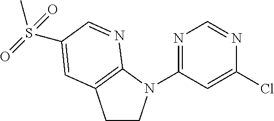

3-Chloroperbenzoic acid (70%, 851 mg, 3.5 mmol) was dissolved in 4 mL of chloroform, and the water that separated was removed. The organic solution was added in one portion to a stirring solution of 1-(6-chloropyrimidin-4-yl)-5-(methylthio)-2,3-dihydro-1H-pyrrolo[2,3-b]pyridine (363 mg, 1.3 mmol) in 8 mL of chloroform. After 1 hour, the excess 3-chloroperbenzoic acid in the reaction was quenched by the addition of dimethyl sulfide. The mixture was stirred for 5 minutes, and then washed with of 0.5 M sodium hydroxide (20 mL). The chloroform layer was separated, washed again with water, and dried over magnesium sulfate. The mixture was filtered, and the filtrate concentrated under reduced pressure to give 1-(6-chloropyrimidin-4-yl)-5-(methylsulfonyl)-2,3-dihydro-1H-pyrrolo[2,3-b]pyridine (366 mg, 90%) as a white powder. ¹H NMR (400 MHz, deuterochloroform): 3.11 (s, 3H), 3.29 (t, J=8.6 Hz, 2H), 4.40-4.49 (m, 2H), 7.92 (d, J=2.1 Hz, 1H), 8.70 (d, J=1.0 Hz, 1H), 8.75 (d, J=2.1 Hz, 1H), 8.86 (d, J=0.8 Hz, 1H). LCMS (ES+): 311 (M+1).

### Preparation 23: Isomers of tert-butyl-3-fluoro-4-hydroxypiperidine-1-carboxylate (4 and 5) The experimental details are described in detail in Scheme B below.

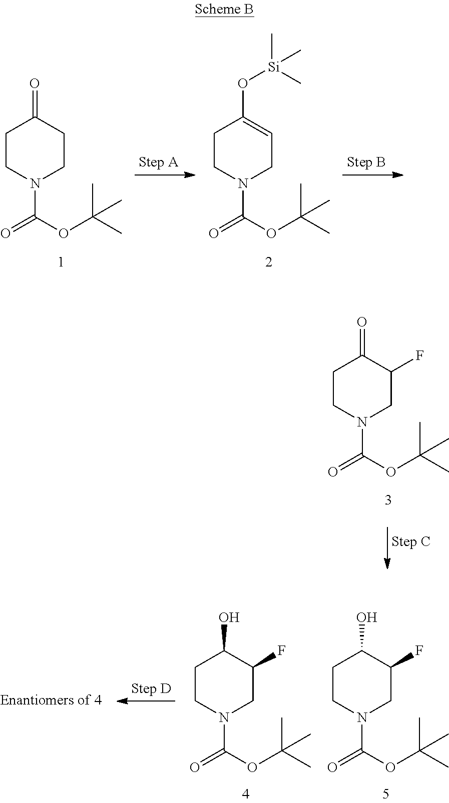

### Step A. tert-Butyl-4-[(trimethylsilyl)oxy]-3,6-dihydropyridine-1(2H)-carboxylate (2)

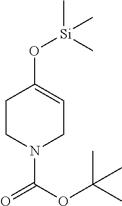

To a solution of N-tert-butoxycarbonyl-4-piperidone (30.0 g, 0.15 mol) in dry N,N-dimethylformamide (300 mL) at room temperature was added trimethylsilyl chloride (22.9 mL, 0.18 mol) and triethylamine (50.4 mL, 0.36 mol) successively via addition funnels. The resulting solution was heated at 80 degrees Celsius overnight and then cooled to room temperature. The reaction mixture was diluted with water and heptane. The layers were separated, and the aqueous layer was extracted with heptane. The combined heptane layers were washed sequentially with water and brine and then dried over magnesium sulfate. The mixture was filtered, and the filtrate concentrated under reduced pressure to give the crude product as a yellow oil. The oil was purified by passing it through a plug of silica gel in 90:10 heptane/ethyl acetate to give the title compound as a colorless oil (33.6 g, 82%). ¹H NMR (400 MHz, deuterochloroform) delta 4.78 (br s, 1H), 3.86 (br s, 2H), 3.51 (t, 2H), 2.09 (br s, 2H), 1.45 (s, 9H), 0.18 (s, 9H).

### Step B. tert-Butyl-3-fluoro-4-oxopiperidine-1-carboxylate (3)

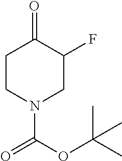

To a stirred solution of tert-butyl-4-[(trimethylsilyl)oxy]-3,6-dihydropyridine-1(2H)-carboxylate (28.8 g, 0.11 mol) in acetonitrile (300 mL) at room temperature was added Selectfluor™ (41.4 g, 0.12 mol). The resulting pale yellow suspension was stirred at room temperature for 1.5 hours. Saturated aqueous sodium bicarbonate (300 mL) and ethyl acetate (300 mL) were added, and the layers were separated. The aqueous layer was extracted twice with ethyl acetate, and all the organic layers were combined and washed sequentially with saturated aqueous sodium bicarbonate and brine and then dried over magnesium sulfate. The mixture was filtered, and the filtrate was concentrated under reduced pressure to give the crude product as a pale yellow oil. Purification of this material by repeated column chromatography on silica gel with heptane/ethyl acetate gradient (2:1-1:1) gave the title compound as a white solid (15.5 g, 67%). ¹H NMR (400 MHz, deuterochloroform): delta 4.88 (dd, 0.5H), 4.77 (dd, 0.5H), 4.47 (br s, 1H), 4.17 (ddd, 1H), 3.25 (br s, 1H), 3.23 (ddd, 1H), 2.58 (m, 1H), 2.51 (m, 1H), 1.49 (s, 9H).

Step B was Also Performed as Follows, Isolating the Hydrate of the Ketone.

To a stirred solution of tert-butyl-4-[(trimethylsilyl)oxy]-3,6-dihydropyridine-1(2H)-carboxylate (41.3 g, 0.15 mol) in acetonitrile (500 mL) at room temperature was added Selectfluor™ (56.9 g, 0.16 mol). The resulting pale yellow suspension was stirred at room temperature for 4 hours 10 minutes. Saturated aqueous sodium bicarbonate and ethyl acetate were added, and the layers were separated. The aqueous layer was extracted twice with ethyl acetate, and all the organic layers were combined and washed sequentially with saturated aqueous sodium bicarbonate and brine and then dried over magnesium sulfate. The mixture was filtered, and the filtrate was concentrated under reduced pressure to give the crude tert-butyl-3-fluoro-4-oxopiperidine-1-carboxylate as white solid. The crude tert-butyl-3-fluoro-4-oxopiperidine-1-carboxylate was suspended in tetrahydrofuran (120 mL) and water (120 mL) was added. The resulting solution was stirred at room temperature for 5.5 hours and then concentrated under reduced pressure. The residue was dried under high vacuum, transferred to an Erlenmeyer flask, and suspended in dichloromethane (250 mL). The resulting suspension was stirred for 5 minutes and the solids collected by filtration using a sintered glass funnel. The resulting filter cake was thoroughly washed with dichloromethane (200 mL), a 1:1 mixture of dichloromethane (200 mL) and heptane (100 mL). The solid was then dried under high vacuum to provide tert-butyl 3-fluoro-4,4-dihydroxypiperidine-1-carboxylate (26.4 g). ¹H NMR (500 MHz, deutero dimethyl sulfoxide) delta 1.38 (s, 9H), 1.49-1.52 (m, 1H), 1.63-1.68 (m, 1H), 2.82-3.20 (m, 2H) 3.75 (br, 1H), 3.97 (br, 1H), 4.12 (d, J=45, 1 H), 5.92 (s, 1H), 5.97 (s, 1H).

### Step C. Isomers of (R*)-tert-butyl-3-(S)-fluoro-4-(R)-hydroxypiperidine-1-carboxylate (4 and 5)(racemic)

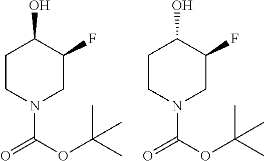

To a solution of tert-butyl-3-fluoro-4-oxopiperidine-1-carboxylate (15.5 g, 71.3 mmol) in methanol (150 mL) at 0 degrees Celsius was added sodium borohydride (3.51 g, 93.7 mmol). The resulting mixture was stirred at 0 degrees Celsius for 2 hours and then allowed to warm to room temperature. Saturated aqueous ammonium chloride (200 mL) was added, and the mixture was extracted three times with ethyl acetate. The combined extracts were washed with brine and dried over magnesium sulfate. The mixture was filtered, and the filtrate was concentrated under reduced pressure to give the crude product mixture which was purified by column chromatography on silica gel eluting with heptane-ethyl acetate (3:2-1:1) to give the first eluting product, tert-butyl-(3,4-trans)-3-fluoro-4-hydroxypiperidine-1-carboxylate (3.81 g, 24%), as a pale yellow oil which solidified on standing to a white solid. ¹H NMR (400 MHz, deuterochloroform) delta 4.35 (ddd, 0.5H), 4.18 (ddd, 0.5H), 4.15 (br s, 1H), 3.89-3.74 (m, 2H), 2.97 (br s, 1H), 2.93 (ddd, 1H), 2.47 (s, 1H), 2.05-1.92 (m, 1H), 1.58-1.46 (m, 1H), 1.44 (s, 9H).

The second eluting compound, tert-butyl-(3,4-cis)-3-fluoro-4-hydroxy-piperidine-1-carboxylate (10.57 g, 68%) was then isolated as a white solid. ¹H NMR (400 MHz, deuterochloroform) delta 4.69-4.65 (m, 0.5H), 4.53-4.49 (m, 0.5H), 3.92-3.86 (m, 2 H), 3.69 (br s, 1H), 3.39 (br s, 1H), 3.16 (br s, 1H), 2.13 (s, 1H), 1.88-1.73 (m, 2H), 1.44 (s, 9H).

Step C was also performed starting with the hydrate tert-butyl 3-fluoro-4,4-dihydroxypiperidine-1-carboxylate as follows.

To a stirred solution of tert-butyl 3-fluoro-4,4-dihydroxypiperidine-1-carboxylate (20.0 g, 85 mmol) in tetrahydrofuran (500 mL) at −35 degrees Celsius was added a solution of L-selectride in tetrahydrofuran (170 mL, 1 M, 170 mmol) drop-wise over 30 minutes. The reaction mixture was warmed to 0 degree Celsius over 1.5 h. The reaction mixture was quenched with saturated aqueous ammonium chloride (150 mL) and vigorously stirred for 15 minutes. To this 0 degree Celsius mixture was added pH 7 phosphate buffer (150 mL), followed by drop-wise addition of a 35% aqueous hydrogen peroxide solution (150 mL). The resulting mixture was stirred for 30 minutes and diluted with ethyl acetate. The organic layer was separated and washed sequentially with water, saturated aqueous sodium thiosulfate and brine. The organic layer was then dried over anhydrous magnesium sulfate, filtered and the filtrate was concentrated under reduced pressure give the crude product mixture which was purified by column chromatography on silica gel [combiflash ISCO 330 g column] eluting with heptane-ethyl acetate (10 to 60% gradient) to give tert-butyl-(3,4-cis)-3-fluoro-4-hydroxypiperidine-1-carboxylate (13.9 g).

### Step D. Enantiomers of tert-butyl-(3,4-cis)-3-fluoro-4-hydroxy-piperidine-1-carboxylate

A 1 gram sample of racemic tert-butyl-(3,4-cis)-3-fluoro-4-hydroxy-piperidine-1-carboxylate was purified into its enantiomers via preparatory high pressure liquid chromatography utilizing a Chiralpak AD-H column (10×250 mm) with a mobile phase of 90:10 carbon dioxide and ethanol respectively at a flow rate of 10 mL/minute. The wavelength for monitoring the separation was 210 nM. The analytical purity of each enantiomer was determined using analytical high pressure chromatography using a Chiralpak AD-H (4.6 mm×25 cm) column with an isocratic mobile phase of 90:10 carbon dioxide and ethanol respectively at a flow rate of 2.5 mL/minute. The wavelength for monitoring the peaks was 210 nm. The following two isomers were obtained:

### (3S,4R)-tert-Butyl 3-fluoro-4-hydroxypiperidine-1-carboxylate, enantiomer 1 (363 mg)

Rₜ=2.67 min (100% ee) (optical rotation in dichloromethane=+21.2 degrees)

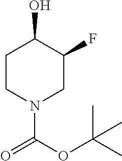

and

### (3R,4S)-tert-Butyl 3-fluoro-4-hydroxypiperidine-1-carboxylate, enantiomer 2 (403 mg)

### Rₜ=2.99 min (88% ee).

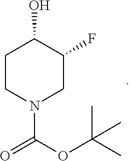

The absolute stereochemistry of the tert-butyl-(3,4-cis)-3-fluoro-4-hydroxy-piperidine-1-carboxylate isomers was determined by making a (1S)-(+)-camphorsulfonic acid salt of 5-(6-((3S,4R)-3-fluoropiperidin-4-yloxy)-5-methylpyrimidin-4-yl)-1-methyl-1,4,5,6-tetrahydropyrrolo[3,4-c]pyrazole (see by analogy the preparation in racemic form below), prepared using enanantiomer 1 above.

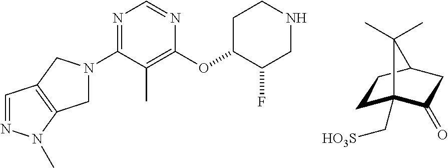

### Preparation of 5-(6-{[3,4-cis)-3-fluoropiperidin-4-yl]oxy}-5-methylpyrimidin-4-yl)-1-methyl-1,4,5,6-tetrahydropyrrolo[3,4-c]pyrazole (racemic)

a. Preparation of 546-Chloro-5-methylpyrimidin-4-yl)-1-methyl-1,4,5,6-tetrahydropyrrolo[3,4-c]pyrazole

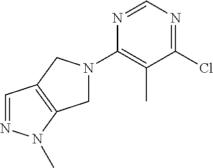

1-Methyl-1,4,5,6-tetrahydropyrrolo[3,4-c]pyrazole bis-hydrochloride salt (2.00 g, 10.2 mmol) and 4,6-dichloro-5-methylpyrimidine (1.66 g, 10.2 mmol) were suspended in tetrahydrofuran (51 mL) at room temperature. To this was added triethylamine (4.41 mL, 31.6 mmol), which caused cloudiness in the mixture and led to a brown solid sticking to the flask walls. This mixture was stirred at room temperature for 4 hours and then heated 50 degrees Celsius for an additional 19 hours. The reaction mixture was cooled to room temperature and diluted with water (100 mL). This mixture was extracted with ethyl acetate (3×100 mL). The organic extracts were pooled, washed with brine, dried over sodium sulfate, and filtered. The filtrate was reduced to dryness under vacuum to yield the title compound as a light brown solid (1.95 g, 78%), which was used in the next step without further purification.

¹H NMR (500 MHz, deuterochloroform) delta 2.54 (s, 3H) 3.88 (s, 3H) 4.90 (app. d, J=3.66 Hz, 4H) 7.28 (s, 1H) 8.29 (s, 1H).

### b. Preparation of tert-Butyl (3,4-cis)-3-fluoro-4-{[5-methyl-6-(1-methyl-4,6-dihydropyrrolo[3,4-c]pyrazol-5(1H)-yl)pyrimidin-4-yl]oxy}piperidine-1-carboxylate (racemic)

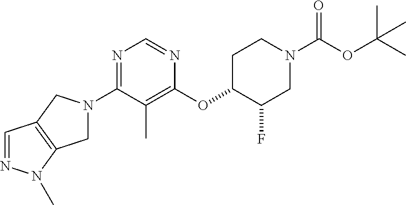

A mixture of tert-butyl (3,4-cis)-3-fluoro-4-hydroxypiperidine-1-carboxylate (1.67 g, 7.62 mmol) and 5-(6-chloro-5-methylpyrimidin-4-yl)-1-methyl-1,4,5,6-tetrahydropyrrolo[3,4-c]pyrazole prepared above (900 mg, 3.60 mmol) was dissolved in 1,4-dioxane (20 mL) and was heated to 105 degrees Celsius. After heating for 10 minutes, all the materials had gone into solution, and sodium bis(trimethylsilyl)amide (4.3 mL, 4.3 mmol, 1M in toluene) was rapidly added to the mixture, resulting in a cloudy yellow mixture that was then stirred for 2 hours at 105 degrees Celsius. The reaction was then cooled to room temperature and quenched by adding an equal volume mixture of water and saturated aqueous sodium bicarbonate solution. The mixture was extracted with ethyl acetate (3×15 mL). The combined organic extracts were washed with brine, dried over sodium sulfate, and filtered. The filtrate was concentrated under vacuum to give a yellow residue that was purified by column chromatography on silica gel eluting with 60 to 100% ethyl acetate in heptane. A mixture of the title compound and the starting 5-(6-chloro-5-methylpyrimidin-4-yl)-1-methyl-1,4,5,6-tetrahydropyrrolo[3,4-c]pyrazole was isolated as a white solid (1.20 g) and was used without further purification in subsequent reactions.

A batch of crude tert-butyl (3,4-cis)-3-fluoro-4-{[5-methyl-6-(1-methyl-4,6-dihydropyrrolo[3,4-c]pyrazol-5(1H)-yl)pyrimidin-4-yl]oxy}piperidine-1-carboxylate from a separate reaction, run under the same conditions, was purified by HPLC. The crude sample (9.5 mg) was dissolved in dimethyl sulfoxide (1 mL) and purified by preparative reverse phase HPLC on a Waters XBridge C₁₈ 19×100 mm, 0.005 mm column, eluting with a linear gradient of 80% water/acetonitrile (0.03% ammonium hydroxide modifier) to 0% water/acetonitrile in 8.5 minutes, followed by a 1.5 minute period at 0% water/acetonitrile; flow rate: 25 mL/minute. The title compound (5 mg) was thus obtained. Analytical LCMS: retention time 2.81 minutes (Waters XBridge C₁₈ 4.6×50 mm, 0.005 mm column; 90% water/acetonitrile linear gradient to 5% water/acetonitrile over 4.0 minutes, followed by a 1 minute period at 5% water/acetonitrile; 0.03% ammonium hydroxide modifier; flow rate: 2.0 mL/minute); LCMS (ES+) 433.2 (M+1).

### c. Preparation of 5-(6-{[(3,4-cis)-3-fluoropiperidin-4-yl]oxy}-5-methylpyrimidin-4-yl)-1-methyl-1,4,5,6-tetrahydropyrrolo[3,4-c]pyrazole (racemic)

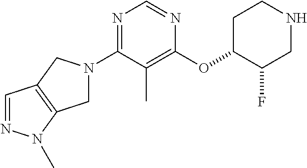

Crude tert-butyl (3,4-cis)-3-fluoro-4-{[5-methyl-6-(1-methyl-4,6-dihydropyrrolo[3,4-c]pyrazol-5(1H)-yl)pyrimidin-4-yl]oxy}piperidine-1-carboxylate (1.20 g) prepared above was dissolved in dichloromethane (12 mL) and to this solution was added trifluoroacetic acid (5 mL). The reaction was stirred at room temperature for 1 hour. The solvent was removed under vacuum, and the residue was dissolved in water (50 mL) and 1N aqueous hydrochloric acid solution (10 mL). The mixture was extracted with dichloromethane (10×30 mL). The aqueous layer was then brought to pH 12 by the addition of 1N aqueous sodium hydroxide solution (20 mL) and was extracted three times with dichloromethane (40 mL). The combined organic extracts were washed with brine, dried over sodium sulfate and filtered. The filtrate was concentrated under reduced pressure to afford 5-(6-{[(3,4-cis)-3-fluoropiperidin-4-yl]oxy}-5-methylpyrimidin-4-yl)-1-methyl-1,4,5,6-tetrahydropyrrolo[3,4-c]pyrazole (0.72 g, 60% over two steps) as a white solid that was used without additional purification.

¹H NMR (500 MHz, deuterochloroform) delta 1.84-2.08 (m, 2H) 2.33 (s, 3H) 2.69-2.84 (m, 1H) 2.83-3.01 (m, 1H) 3.16 (d, J=13.66 Hz, 1H) 3.27-3.44 (m, 1H) 3.86 (s, 3H) 4.78-4.91 (m, 1H) 4.86 (d, J=1.95 Hz, 2H) 4.88 (d, J=1.95 Hz, 2H) 5.21-5.32 (m, 1H) 7.26 (s, 1H) 8.18 (s, 1H); LCMS (ES+) 333.4 (M+1).

tert-Butyl-(3,4-cis)-3-fluoro-4-hydroxy-piperidine-1-carboxylate in racemic form was also prepared as follows:

To a Biotage Atlantis reactor was added 3-fluoropyridin-4-ol (2.0 g, 17.7 mmol), hexamethyldisilazane (3.7 mL, 17.7 mmol) and 20 mL of tetrahydrofuran. The reactor was purged with nitrogen gas (4×), pressurizing to 50 psi, followed by venting. The mixture was stirred at 1000 rpm while heating to 80 degrees Celsius. The mixture was heated at 80 degrees Celsius for 1 hour before cooling the mixture to room temperature. Di-tert-butyldicarbonate (7.7 g, 35.4 mmoles) and ruthenium (400 mg, 5% on carbon, 198 micromoles, JM UK-35) were added. The reactor was purged with nitrogen gas (4×) and then with hydrogen gas (4×). The mixture was heated to 105 degrees Celsius, pressurized to 200 psi with hydrogen gas for 24 hours. The mixture was cooled to 30 degrees Celsius and purged with nitrogen gas (4×). The mixture was filtered and washed with tetrahydrofuran. GCMS analysis of the filtrate showed 89% of tert-butyl 3-fluoro-4-hydroxypiperidine-1-carboxylate.

### Preparation 24: 1-Methylcyclopropyl 4-nitrophenyl carbonate

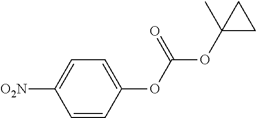

### Step A) 1-Methylcyclopropanol

A 1 L flask was charged with titanium methoxide (100 g), cyclohexanol (232 g), and toluene (461 mL). The flask was equipped with a Dean-Stark trap and condenser. The mixture was heated at 140 degrees Celsius until the methanol was removed. The toluene was removed at 180 degrees Celsius. More toluene was added and this process was repeated twice. After all the toluene was removed the flask was dried under high vacuum. Diethyl ether (580 mL) was added to the flask to prepare a 1 M solution in diethyl ether. A 5 L, 3-neck flask was equipped with an overhead stirrer, inert gas inlet and a pressure-equalizing addition funnel. The flask was flushed with nitrogen gas and charged with methyl acetate (60.1 mL, 756 mmol), titanium cyclohexyloxide (1 M solution in ether 75.6 mL), and diethyl ether (1500 mL). The solution was stirred while keeping the reaction flask in a room temperature water bath. The addition funnel was charged with the 3 M ethylmagnesium bromide solution (554 mL, 1.66 moles). The Grignard reagent was added drop-wise over 3 hours at room temperature. The mixture became a light yellow solution, and then gradually a precipitate formed which eventually turned to a dark green/brown/black colored mixture. After stirring for an additional 15 minutes, following the addition of the Grignard, the mixture was carefully poured into a mixture of 10% concentrated sulfuric acid in 1 L of water. The resulting mixture was stirred until all the solids dissolved. The aqueous layer was separated and extracted with diethyl ether 2×500 mL. The combined organic extracts were washed sequentially with water, brine, dried over potassium carbonate (500 g) for 30 minutes, filtered and the filtrate was concentrated in vacuo to an oil. Sodium bicarbonate (200 mg) was added and the crude material was distilled, collecting fractions boiling around 100 degrees Celsius to give the title compound (23 grams) with methyl ethyl ketone and 2-butanol as minor impurities. ¹H NMR (500 MHz, deuterochloroform) delta 0.45 (app. t, J=6.59 Hz, 2H), 0.77 (app. t, J=5.61 Hz, 2H), 1.46 (s, 3H). The preparation of the title compound is also described in WO09105717.

### Step B) 1-Methylcyclopropyl 4-nitrophenyl carbonate

A solution of 1-methylcyclopropanol (10 g, 137 mmol), 4-nitrophehyl chloroformate (32 g, 152 mmol), and a few crystals of 4-dimethylaminopyridine (150 mg, 1.2 mmol) in dichloromethane (462 mL), was cooled to zero degree Celsius.

Triethylamine (36.5 g, 361 mmol) was added drop-wise. After 10 minutes, the ice bath was removed and the reaction was allowed to stir at room temperature for 14 hours. The reaction mixture was washed twice with saturated aqueous sodium carbonate. The aqueous phase was extracted with dichloromethane. The combined organic extracts were washed with water, dried over magnesium sulfate, filtered and the filtrate concentrated in vacuo. The residue was purified by flash silica gel chromatography, eluting with a gradient mixture of ethyl acetate in heptane (0 to 5% ethyl acetate over the first 10 minutes, then isocratic at 5% ethyl acetate to heptane) to give 20.8 g of the desired carbonate as a clear oil. This oil solidified upon standing.

¹H NMR (500 MHz, deuterochloroform) delta 0.77 (app. t, J=6.59 Hz, 2H), 1.09 (app. t, J=7.07 Hz, 2H), 1.67 (s, 3H), 7.40 (app. dt, J=9.27, 3.17 Hz, 2H), 8.29 (app. dt, J=9.27, 3.17 Hz, 2H).

Alternatively the 1-methylcyclopropanol can be prepared as follows:

### 1-Methylcyclopropanol

A 2000 mL 4-neck flask was equipped with a mechanical stirrer, inert gas inlet, thermometer, and two pressure-equalizing addition funnels. The flask was flushed with nitrogen and charged with 490 mL of diethyl ether followed by 18.2 mL (30 mmol) of titanium tetra(2-ethylhexyloxide). One addition funnel was charged with a solution prepared from 28.6 mL (360 mmol) of methyl acetate diluted to 120 mL with ether. The second addition funnel was charged with 200 mL of 3 M ethylmagnesium bromide in ether solution. The reaction flask was cooled in an ice water bath to keep the internal temperature at 10 degrees Celsius or below. Forty milliliters of the methyl acetate solution was added to the flask. The Grignard reagent was then added drop-wise from the addition funnel at a rate of about 2 drops every second, and no faster than 2 mL per minute. After the first 40 mL of Grignard reagent had been added, another 20 mL portion of methyl acetate in ether solution was added. After the second 40 mL of Grignard reagent had been added, another 20 mL portion of methyl acetate in diethyl ether solution was added. After the third 40 mL of Grignard reagent had been added, another 20 mL portion of methyl acetate in ether solution was added. After the fourth 40 mL of Grignard reagent had been added, the last 20 mL portion of methyl acetate in ether solution was added. The mixture was stirred for an additional 15 minutes following the completion of the addition of Grignard reagent. The mixture was then poured into a mixture of 660 g of ice and 60 mL of concentrated sulfuric acid with rapid stirring to dissolve all solids. The phases were separated and the aqueous phase was extracted again with 50 mL of diethyl ether. The combined ether extracts were washed with 15 mL of 10% aqueous sodium carbonate, 15 mL of brine, and dried over 30 grams magnesium sulfate for 1 hour with stirring. The ether solution was then filtered. Tri-n-butylamine (14.3 mL, 60 mmol) and mesitylene (10 mL) were added. Most of the diethyl ether was removed by distillation at atmospheric pressure using a 2.5 cm×30 cm jacketed Vigreux column. The remaining liquid was transferred to a smaller distillation flask using two 10 mL portions of hexane to facilitate the transfer. Distillation at atmospheric pressure was continued through a 2 cm×20 cm jacketed Vigreux column. The liquid distilling at 98-105° C. was collected to provide 14 g of the title compound as a colorless liquid. ¹H NMR (400 MHz, deuterochloroform) delta 0.42-0.48 (m, 2H), 0.74-0.80 (m, 2H), 1.45 (s, 3H), 1.86 (br. s., 1H).

### Preparation 25: 1-Methylcyclobutyl 4-nitrophenyl carbonate

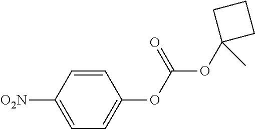

### Step A: 1-Methylcyclobutanol

To a solution of magnesium bromide ethyl etherate complex (4.24 g, 16.4 mmol) in diethylether (71 mL) at −78 degrees Celsius was added methyllithium (9.81 mL, 15.7 mmol, 1.6 M in diethylether). The mixture was stirred for 15 minutes whereupon cyclobutanone (1.1 mL, 14 mmol) was added drop-wise. The mixture was stirred for 2 hours at −78 degrees Celsius before the reaction was quenched with 1.0 M aqueous hydrochloric acid (16 mL). The mixture was warmed to room temperature over 1 hour before the pH was made slightly alkaline with 1.0 M aqueous sodium hydroxide. The aqueous layer was separated and extracted with diethylether (2×40 mL). The combined organic layers were washed with water (50 mL), dried over sodium sulfate, filtered, and the filtrate was concentrated in vacuo to give 1-methylcyclobutanol which was used in the subsequent step without purification.

### Step B: 1-Methylcyclobutyl 4-nitrophenyl carbonate

To a stirred solution of the crude 1-methylcyclobutanol (1.20 g, 13.9 mmol) and pyridine (1.34 mL, 16.7 mmol) in dichloromethane (46 mL) was added the 4-nitrophenyl carbonochloridate (3.37 g, 16.7 mmol) portion-wise over 10 minutes at 0 degrees Celsius. The mixture was warmed to room temperature over 3 hours. The reaction was quenched with water and the aqueous layer was extracted with dichloromethane (3×). The combined organic layers were dried over sodium sulfate, filtered, and the filtrate was concentrated in vacuo. The crude residue was purified by ISCO MPLC (0-20% ethyl acetate in heptane) to afford 1-methylcyclobutyl 4-nitrophenyl carbonate (1.67 g, 48% over 2 steps) as a clear oil. ¹H NMR (500 MHz, deuterochloroform): delta 8.29 (m, 2H), 7.40 (m, 2H), 2.53-2.44 (m, 2H), 2.26-2.19 (m, 2H), 1.96-1.86 (m, 1H), 1.77-1.67 (m, 1H), 1.67 (s, 3H).

### Preparation 26: 1-Ethylcyclopropyl 4-nitrophenyl carbonate

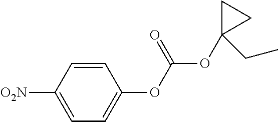

### Step A: 1-ethylcyclopropanol

To a stirred solution of chloroiodomethane (11.6 g, 66 mmol), propionyl chloride (2.78 g, 30 mmol) and lithium bromide (5.79 g, 66 mmol) in tetrahydrofuran (120 mL) was added a solution of methyllithium (1.6 M in diethyl ether, 41.2 mL, 66 mmol) over 20 minutes at −78 degrees Celsius (bath temperature) under a nitrogen atmosphere. The reaction mixture was stirred at −78 degrees Celsius for 3 hours. Lithium powder (5.93 g, 270 mmol) was then added cautiously and the mixture was stirred for 16 hours, allowing the temperature to rise slowly to room temperature. The mixture was then cooled to 0 degrees Celsius and diluted with water (145 mL) and concentrated hydrochloric acid (30 mL). The aqueous mixture was extracted with diethyl ether (3×200 mL). The combined organic extracts were dried over magnesium sulfate, filtered and the filtrate was concentrated to give 1-ethylcyclopropanol as a yellow oil (2.5 g). This material was used in the next step without further purification. ¹H NMR (500 MHz, deuterochloroform) delta 0.37-0.43 (m, 2H) 0.65-0.73 (m, 2H) 1.01 (t, J=7.44 Hz, 3H) 1.56 (q, J=7.48 Hz, 2H).

### Step B: 1-ethylcyclopropyl 4-nitrophenyl carbonate

To the crude 1-ethylcyclopropanol (1.0 g, 11.6 mmol) and pyridine (1.12 mL, 13.9 mmol) in dichloromethane (5 mL) at 0 degrees Celsius was added the 4-nitrophehyl chloroformate (2.81 g, 13.9 mmol) portion-wise over 10 minutes. The ice bath was allowed to warm and the mixture was stirred at room temperature for 18 hours. The reaction was quenched with water and the mixture was extracted with dichloromethane (3×). The combined organic extracts were dried over magnesium sulfate, filtered and the filtrate was concentrated in vacuo. The residue was purified by chromatography with a 40 g silica gel column, eluting with a gradient mixture of ethyl acetate and heptane from 5% to 25% to give a yellow oil (1.0 g). This material was further purified by HPLC (conditions: column, Chiralpak AD-H; solvent, methanol; flow, 10.0 mL/minute; wavelength, 210 nm), to give 450 mg of 1-ethylcyclopropyl 4-nitrophenyl carbonate as a pale yellow oil. ¹H NMR (500 MHz, deuterochloroform) delta 0.75-0.81 (m, 2H) 1.01-1.09 (m, 5H) 1.92 (q, J=7.48 Hz, 2H) 7.36-7.42 (m, 2H) 8.24-8.30 (m, 2H).

### Preparation 27: tert-Butyl 4-[(6-chloro-5-methylpyrimidin-4-yl)oxy]piperidine-1-carboxylate

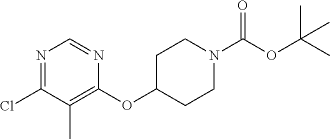

A 20 mL Biotage™ microwave tube was purged with nitrogen and charged with 4,6-dichloro-5-methylpyrimidine (0.600 g, 2.98 mmol) and tert-butyl 4-hydroxypiperidine-1-carboxylate (534 mg, 3.28 mmol). 1,4-Dioxane (14.9 mL) was added, and the mixture was heated to 100 degrees Celsius. To the mixture was added sodium bis(trimethylsilyl)amide (3.58 mL, 3.58 mmol, 1.0 M in tetrahydrofuran) drop-wise over 10 minutes. The mixture was stirred for 60 minutes, and then at room temperature for 12 hours. The reaction was quenched with water, and the aqueous layer was extracted three times with ethyl acetate. The combined organic extracts were dried over sodium sulfate, filtered, and the filtrate was concentrated in vacuo. The crude material was purified via silica gel chromatography (40 g SiO₂ column, 0-50% ethyl acetate in heptane gradient) to afford the title compound (842 mg, 86%).

### Preparation 28: tert-Butyl (3R,4S)-4-[(6-chloro-5-methylpyrimidin-4-yl)oxy]-3-fluoropiperidine-1-carboxylate (racemic)

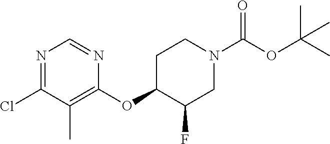

To a solution of tert-butyl-(3,4-cis)-3-fluoro-4-hydroxy-piperidine-1-carboxylate (racemic) (1.0 g, 4.6 mmol) and 4,6-dichloro-5-methylpyrimidine (818 mg, 5.02 mmol) in anhydrous tetrahydrofuran (23 mL) was added sodium hydride (201 mg, 5.02 mmol, 60% dispersion in mineral oil) in two portions at 0 degrees Celsius. After 18 hours, the reaction mixture was quenched with saturated aqueous ammonium chloride and diluted with water. The resulting mixture was extracted three times with ethyl acetate. The combined organic layers were dried over sodium sulfate, filtered, and the filtrate was concentrated under reduced pressure to afford the title compound as a pale yellow oil (1.56 g, 99%). ¹H NMR (400 MHz, deuterochloroform) delta 1.46 (s, 9H), 1.84-1.91 (m, 1H), 2.04-2.17 (m, 1H), 2.24 (s, 3H), 3.09-3.22 (m, 1H), 3.29-3.43 (m, 1H), 3.78-4.01 (m, 1H), 4.09-4.20 (m, 1H), 4.74-4.93 (m, 1H), 5.31-5.43 (m, 1H), 8.36 (s, 1H). LCMS: (ES+): 346.4 (M+1).

### Preparation 29: 4-Chloro-6-{[(3R,4S)-3-fluoropiperidin-4-yl]oxy}-5-methylpyrimidine

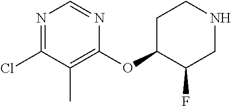

To a solution of tert-butyl (3R,4S)-4-[(6-chloro-5-methylpyrimidin-4-yl)oxy]-3-fluoropiperidine-1-carboxylate (1.4 g, 4.0 mmol) in anhydrous 1,2-dichloroethane (20 mL) was added trifluoroacetic acid (4.0 mL, 52.0 mmol) at room temperature under a positive stream of nitrogen. After 2 hours, the volatiles were removed under reduced pressure and heat to afford a colorless residue. The residue was taken up dichloromethane and basified with saturated aqueous sodium bicarbonate. The mixture was then extracted three times with dichloromethane. The combined organic layers were dried over sodium sulfate, filtered, and concentrated under reduced pressure to afford product as an off-white solid (930 mg, 93%). ¹H NMR (400 MHz, deuterochloroform) delta 1.88-2.07 (m, 2H) 2.25 (s, 3H) 2.73-2.82 (m, 1H) 2.86-2.99 (m, 1H) 3.12-3.20 (m, 1H) 3.31-3.39 (m, 1H) 4.76-4.93 (m, 1H) 5.24-5.37 (m, 1H) 8.36 (s, 1H) LCMS: (ES+): 246.2 (M+1).

### Preparation 30:1-Methylcyclopropyl (3R,4S)-4-[(6-chloro-5-methylpyrimidin-4-yl)oxy]-3-fluoropiperidine-1-carboxylate

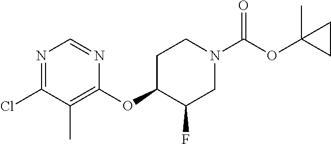

To a solution of 4-chloro-6-{[(3R,4S)-3-fluoropiperidin-4-yl]oxy}-5-methylpyrimidine (925 mg, 3.76 mmol) and triethylamine (1.57 mL, 11.3 mmol) in dichloromethane (20.0 mL) was added 1-methylcyclopropyl 4-nitrophenyl carbonate (1.79 mg, 7.53 mmol) at room temperature. After 72 hours, the reaction was quenched with water and extracted three times with dichloromethane. The combined organic layers were washed continuously with a solution of saturated aqueous sodium bicarbonate until the yellow color was removed. Then the organic layer was dried over sodium sulfate, filtered, and concentrated under reduced pressure. The resulting crude residue was purified by flash chromatography (silica: 10-50% ethyl acetate: heptane) to afford 830 mg (64%) of desired product as a white solid. ¹H NMR (500 MHz, deuterochloroform) delta 0.63-0.68 (m, 2H), 0.87-0.94 (m, 2H), 1.60 (s, 3H), 1.86-1.97 (m, 1H), 2.08-2.19 (m, 1H), 2.27 (s, 3H), 3.11-3.27 (m, 1H) 3.27-3.49 (m, 1 H), 3.78-4.11 (m, 1H), 4.11-4.27 (m, 1H), 4.77-4.96 (m, 1H), 5.33-5.46 (m, 1H), 8.40 (s, 1H) LCMS: (ES+): 344.4 (M+1).

### Preparation 31: 6,7-Dihydro-5H-pyrrolo[3,2-c]pyridazine

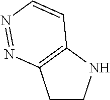

### Step A: Benzyl 3-oxo-4,4-a,6,7-tetrahydro-2H-pyrrolo[3,2-c]pyridazine-5(3H)-carboxylate

A solution of benzyl 2-(2-ethoxy-2-oxoethyl)-3-oxopyrrolidine-1-carboxylate (prepared as described in Synlett 1998, 1378) (6.47 g, 21.2 mmol) in ethanol (66 mL), acetic acid (11 mL) and hydrazine (0.73 mL, 23.3 mmol) was heated at reflux (100 degrees Celsius) for 6 hours. The mixture was cooled to room temperature and concentrated in vacuo to give 8.13 g of a dark brown viscous oil. This crude material was purified by silica gel chromatography, eluting with a gradient mixture of ethyl acetate and heptane 40% to 90% ethyl acetate to give to give benzyl 3-oxo-4,4-a,6,7-tetrahydro-2H-pyrrolo[3,2-c]pyridazine-5(3H)-carboxylate (3.70 g, 64%) as a light tan foam. ¹H NMR (400 MHz, deuterochloroform) delta 2.29 (t, J=15.34 Hz, 1H) 2.65-2.80 (m, 1H) 2.80-2.97 (m, 1H) 3.05-3.52 (m, 1H) 3.65 (br. d, J=6.60 Hz, 1H) 4.04 (br. s., 1H) 4.49 (br. s., 1H) 5.08-5.27 (m, 2H) 7.38 (s, 4H) 8.27 (br. s., 1H).

### Step B: Benzyl 3-oxo-6,7-dihydro-2H-pyrrolo[3,2-d]pyridazine-5(3H)-carboxylate

A mixture of benzyl 3-oxo-4,4-a,6,7-tetrahydro-2H-pyrrolo[3,2-c]pyridazine-5(3H)-carboxylate (3.65 g, 13.3 mmol) and copper (II) chloride (3.59 g, 26.7 mmol) in acetonitrile (53 mL) was heated to 100 degrees Celsius for 1 hour. The mixture was cooled to room temperature then poured into 150 mL water. The aqueous mixture was stirred for 15 minutes, and the solids were collected by filtration through a Pall GHP membrane (0.45 micrometer), and dried under vacuum to give benzyl 3-oxo-6,7-dihydro-2H-pyrrolo[3,2-c]pyridazine-5(3H)-carboxylate (2.06 g, 57%) as an off-white solid. This material was used in the subsequent step without purification. ¹H NMR (400 MHz, deuteromethanol) delta 3.04 (t, J=8.21 Hz, 2H) 4.07 (t, J=8.11 Hz, 2H) 5.29 (s, 2 H) 6.81 (br. s, 1H) 7.19-7.60 (m, 5H).

### Step C: Benzyl 3-chloro-6,7-dihydro-5H-pyrrolo[3,2-d]pyridazine-5-carboxylate

A mixture of benzyl 3-oxo-6,7-dihydro-2H-pyrrolo[3,2-c]pyridazine-5(3H)-carboxylate (2.06 g, 2.47 mmol) and phosphorus oxychloride (22.5 mL) was heated at 110 degrees Celsius for 20 minutes. The excess phosphorus oxychloride was removed in vacuo; the dark blackish-blue residue was diluted with 70 mL water and extracted with dichlormethane (3×25 mL). The combined organic extracts were dried (sodium sulfate), filtered and the filtrate was concentrated in vacuo. The residue (2.22 g of a dark blue solid) was purified by silica gel chromatography, eluting with a gradient mixture of ethyl acetate and heptane 25% to 70% ethyl acetate to give benzyl 3-chloro-6,7-dihydro-5H-pyrrolo[3,2-c]pyridazine-5-carboxylate (1.8385 g, 84%) as a tan solid. MS: ES+: 290.0. ¹H NMR (400 MHz, deuterochlorform) delta 3.43 (t, J=8.79 Hz, 2H) 4.17 (t, J=8.70 Hz, 2H) 5.25-5.40 (m, 2H) 7.35-7.47 (m, 6H).

### Step D: 6,7-dihydro-5H-pyrrolo[3,2-c]pyridazine

A mixture of benzyl 3-chloro-6,7-dihydro-5H-pyrrolo[3,2-c]pyridazine-5-carboxylate (320 mg, 1.1 mmol), Pd/C (10% wt., 59 mg) and ethanol (14 mL) was shaken under a hydrogen atmosphere (50 psi) at room temperature for 16 hours. The Pd/C catalyst was removed by filtration and the filtrate was concentrated in vacuo to give 175 mg of a tan solid. This solid was dissolved in methanol (10 mL) and water (1 mL) and potassium bicarbonate (220 mg, 2.2 mmol) was added. The mixture was stirred for one hour before the solids were removed by filtration and the filtrate was concentrated in vacuo. The residue was dissolved in methanol, stirred with basic alumina (2 g) for 30 minutes, and the solvent was removed in vacuo. This solid was placed atop a column of basic alumina and eluted with a gradient mixture of methanol and dichloromethane 0 to 5% methanol to give 112 mg (84%) of 6,7-dihydro-5H-pyrrolo[3,2-c]pyridazine as a yellow solid. ¹H NMR (400 MHz, deuteromethanol) delta 3.23 (t, J=8.50 Hz, 2H) 3.71 (t, J=8.50 Hz, 2H) 6.40 (d, J=5.86 Hz, 1H) 8.24 (d, J=5.86 Hz, 1H).

### Preparation 32: 5-(6-Chloro-5-methylpyrimidin-4-yl)-6,7-dihydro-5H-pyrrolo[3,2-c]pyridazine

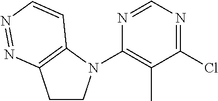

A mixture of 4,6-dichloro-5-methylpyrimidine (19 mg, 0.12 mmol), 6,7-dihydro-5H-pyrrolo[3,2-c]pyridazine (14 mg, 0.12 mmol) and cesium carbonate (38 mg, 0.12 mmol) in N,N-dimethylformamide (0.2 mL) was stirred at room temperature for 40 hours. This reaction mixture was combined with a reaction mixture of an experiment carried out using 4,6-dichloro-5-methylpyrimidine (84 mg, 0.52 mmol), 6,7-dihydro-5H-pyrrolo[3,2-c]pyridazine (74 mg, 0.47 mmol), and cesium carbonate (172 mg, 0.52 mmol) in N,N-dimethylformamide (1 mL) that had been stirred for 15 hours at room temperature. The combined mixture was diluted with water (25 mL) and extracted three times with ethyl acetate. The combined organic extracts were washed with brine, dried (sodium sulfate), filtered and the filtrate was concentrated in vacuo. The residue was purified by silica gel chromatography, eluting with a gradient mixture of methanol and dichloromethane from 0 to 5% methanol, to give 79 mg (54%) of 5-(6-chloro-5-methylpyrimidin-4-yl)-6,7-dihydro-5H-pyrrolo[3,2-c]pyridazine as a tan solid. ¹H NMR (400 MHz, deuterochloroform) delta 2.32 (s, 3H) 3.55 (t, J=8.39 Hz, 2H) 4.29 (t, J=8.39 Hz, 2H) 6.72 (d, J=5.66 Hz, 1H) 8.63 (s, 1H) 8.77 (d, J=5.66 Hz, 1H).

### Preparation 33: 1-Methylcyclopropyl 4-(6-chloropyrimidin-4-yloxy)piperidine-1-carboxylate

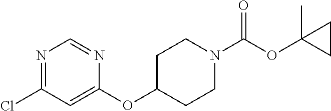

1-Methylcyclopropyl 4-(6-chloropyrimidin-4-yloxy)piperidine-1-carboxylate was prepared in a manner analogous to isopropyl 4-[(6-chloro-pyrimidin-4-yl)oxy]piperidine-1-carboxylate. ¹H NMR (500 MHz, deuterochloroform) delta 0.51-0.74 (m, 2H) 0.77-1.00 (m, 2H) 1.57 (s, 3H) 1.74 (br. s., 2H) 1.98 (br. s., 2H) 3.31 (br. s., 2H) 3.78 (br. s, 2H) 5.28-5.37 (m, 1H) 6.76 (s, 1H) 8.55 (s, 1H).

### Preparation 34: (3R,4S)-1-Methylcyclopropyl 4-(6-chloropyrimidin-4-yloxy)-3-fluoropiperidine-1-carboxylate (racemic)

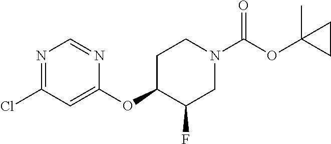

(3R,4S)-1-Methylcyclopropyl 4-(6-chloropyrimidin-4-yloxy)-3-fluoropiperidine-1-carboxylate (racemic) was prepared in a manner analogous to isopropyl 4-[(6-chloro-pyrimidin-4-yl)oxy]piperidine-1-carboxylate. ¹H NMR (deuterochloroform) delta 8.52 (d, J=0.8 Hz, 1H), 6.83 (d, J=0.8 Hz, 1H), 5.20-5.52 (m, 1H), 4.68-5.02 (m, 1H), 3.72-4.31 (m, 2H), 2.93-3.44 (m, 2H), 1.97-2.22 (m, 1H), 1.88 (br. s., 1H), 1.54 (s, 3H), 0.75-0.97 (m, 2H), 0.52-0.70 (m, 2H).

### Preparation 35: Methyl 2,3-dihydro-1H-pyrrolo[3,2-b]pyridine-5-carboxylate and 1-tert-butyl 5-methyl 2,3-dihydro-1H-pyrrolo[3,2-b]pyridine-1,5-dicarboxylate

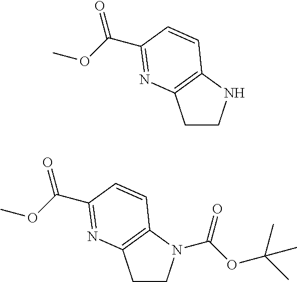

A solution of methyl 1H-pyrrolo[3,2-b]pyridine-5-carboxylate (Adesis Inc., New Castle, Del.) (1.0 g, 5.68 mmol) and di-tert-butyl dicarbonate (1.75 g, 7.95 mmol) in methanol (30 mL) was passed through an H-cube hydrogenation apparatus equipped with a 10%Pd/C cartrige at 80 degrees Celsius and 80 bar 1.0 mL/minute. The effluent was then passed through the H-cube apparatus three additional times. The crude material was concentrated and the residue was purified by silica gel chromatography, eluting with a gradient mixture of 50% to 90% ethyl acetate to heptane to give 230 mg of methyl 2,3-dihydro-1H-pyrrolo[3,2-b]pyridine-5-carboxylate and 300 mg of 1-tert-butyl 5-methyl 2,3-dihydro-1H-pyrrolo[3,2-b]pyridine-1,5-dicarboxylate.

Methyl 2,3-dihydro-1H-pyrrolo[3,2-b]pyridine-5-carboxylate:_{—}¹H NMR (deuterochloroform) delta 7.77 (d, J=8.2 Hz, 1H), 6.67 (d, J=8.2 Hz, 1H), 4.42 (br. s., 1H), 3.88 (s, 3H), 3.69 (td, J=8.6, 1.5 Hz, 2H), 3.17 (t, J=8.7 Hz, 2H).

1-tert-Butyl 5-methyl 2,3-dihydro-1H-pyrrolo[3,2-b]pyridine-1,5-dicarboxylate: ¹H NMR (deuteurochloroform) delta 7.92 (d, J=8.2 Hz, 2H), 4.00 (t, J=8.9 Hz, 2H), 3.91 (s, 3H), 3.25 (t, J=1.0 Hz, 2H), 1.52 (br. s., 9H).

### Preparation 36: N,N-Dimethyl-2,3-dihydro-1H-pyrrolo[3,2-b]pyridine-5-carboxamide

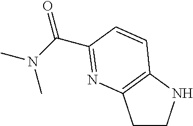

### Step A: 1-(tert-Butoxycarbonyl)-2,3-dihydro-1H-pyrrolo[3,2-b]pyridine-5-carboxylic acid

To a stirred solution of 1-tert-butyl 5-methyl 2,3-dihydropyrrolo[3,2-b]pyridine-1,5-dicarboxylate (250 mg, 0.898 mmol) in a solution of tetrahydrofuran and water (3:1, 4 mL) was added lithium hydroxide monohydrate (59 mg, 1.35 mmol). The reaction mixture was stirred at room temperature for 18 hours before 1 N aqueous hydrochloric acid was added until the solution was approximately pH 2. The mixture was extracted twice with ethyl acetate, dried over magnesium sulfate, filtered and the filtrate was concentrated to give 1-(tert-butoxycarbonyl)-2,3-dihydro-1H-pyrrolo[3,2-b]pyridine-5-carboxylic acid as a pink solid (240 mg). This material was used in the next step without purification. ¹H NMR (deuterochloroform) delta 7.91-8.20 (m, 2H), 4.01-4.10 (m, 2H), 3.25 (t, J=1.0 Hz, 2H), 1.48-1.67 (m, 9H).

### Step B: tert-Butyl 5-(dimethylcarbamoyl)-2,3-dihydro-1H-pyrrolo[3,2-b]pyridine-1-carboxylate

To a stirred solution of 1-(tert-butoxycarbonyl)-2,3-dihydro-1H-pyrrolo[3,2-b]pyridine-5-carboxylic acid (120 mg, 0.45 mmol) in dichloromethane (3 mL) was added N-(3-dimethylaminopropyl)-W-ethylcarbodiimide hydrochloride (131 mg, 0.68 mmol) and 1-hydroxybenzotriazole hydrate (104 mg, 0.68 mmol). The resulting mixture was stirred for 5 minutes, before dimethylamine (2 M in tetrahydrofuran, 0.91 mL, 1.82 mmol) was added. The resulting solution was stirred at room temperature for 18 hours and at 50 degrees Celsius for 6 hours. The mixture was then cooled to room temperature and diluted with dichloromethane. The organic mixture was washed with saturated aqueous sodium bicarbonate and brine. The organic layer was dried over sodium sulfate, filtered and the filtrate was concentrated in vacuo. The residue was purified by silica gel chromatography, eluting with a gradient mixture of dichlormethane and methanol from 0% to 5% methanol to give tert-butyl 5-(dimethylcarbamoyl)-2,3-dihydro-1H-pyrrolo[3,2-b]pyridine-1-carboxylate as a white solid (60 mg). MS (m/z): 292.1 (M+1). ¹H NMR (deuterochloroform) delta 7.49-8.12 (m, 1H), 7.41 (d, J=8.4 Hz, 1H), 4.01 (t, J=8.8 Hz, 2H), 3.21 (t, J=8.9 Hz, 2H), 3.08 (br. s., 6H), 1.54 (br. s., 9H).

### Step C: N,N-Dimethyl-2,3-dihydro-1H-pyrrolo[3,2-b]pyridine-5-carboxamide

To a stirred solution of tert-butyl 5-(dimethylcarbamoyl)-2,3-dihydropyrrolo[3,2-b]pyridine-1-carboxylate (60 mg, 0.26 mmol) in dichloromethane (0.5 mL), was added trifluoroacetic acid (0.5 mL). The resulting solution was stirred at room temperature for 2 hours. The reaction mixture was concentrated in vacuo and the residue was dried under high vacuum to give N,N-dimethyl-2,3-dihydro-1H-pyrrolo[3,2-b]pyridine-5-carboxamide as a pink solid (39 mg). This material was used without purification.

### EXAMPLES

### Example 1

### Isopropyl 9-anti-({6-[5-(methylsulfonyl)-2,3-dihydro-1H-indol-1-yl]pyrimidin-4-yl}oxy)-3-oxa-7-azabicyclo[3.3.1]nonane-7-carboxylate

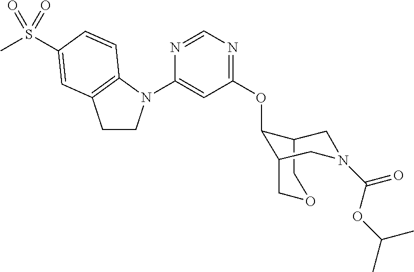

Isopropyl 9-anti-hydroxy-3-oxa-7-azabicyclo[3.3.1]nonane-7-carboxylate (30 mg, 0.13 mmol) and 1-(6-chloropyrimidin-4-yl)-5-(methylsulfonyl)indoline (34 mg, 0.11 mmol) were dissolved in anhydrous 1,4-dioxane (1 mL). The brown mixture was heated at 105 degrees Celsius, and a 1 M solution of sodium bis(trimethylsilyl)amide in tetrahydrofuran (0.13 mL, 0.13 mmol) was added. The mixture was heated at 105 degrees Celsius for 1.5 hours, and then the mixture was allowed to cool to room temperature. The reaction was quenched with 10% aqueous phosphoric acid (0.5 mL). The organic solvents were concentrated under reduced pressure, and the resulting residue was partitioned between chloroform and water. The layers were separated, and the organic layer washed sequentially with water and brine and then dried over magnesium sulfate. The mixture was filtered, and the filtrate concentrated under reduced pressure to give a brown foam. Purification of the crude material by column chromatography (0-10% acetone in dichloromethane) provided isopropyl 9-anti-({6-[5-(methylsulfonyl)-2,3-dihydro-1H-indol-1-yl]pyrimidin-4-yl}oxy)-3-oxa-7-azabicyclo[3.3.1]nonane-7-carboxylate as a white solid (30 mg, 54%). ¹H NMR (400 MHz, deuterochloroform) delta 1.25 (d, J=5.2 Hz, 3H), 1.26 (d, J=5.2 Hz, 3H), 2.01-2.06 (m, 2H), 3.04 (s, 3H), 3.32 (m, 2H), 3.41 (d, J=13.4 Hz, 1H), 3.48 (d, J=13.4 Hz, 1H), 3.85 (m, 2H), 4.06-4.20 (m, 5H), 4.29 (d, J=13.4 Hz, 1H), 4.97 (m, 1H), 5.41 (br. s., 1H), 6.05 (s, 1H), 7.72 (s, 1H), 7.79 (d, J=8.4 Hz 1H), 8.50 (s, 1H), 8.59 (d, J=8.4 Hz, 1H); LCMS (ES+) 503.3 (M+1).

### Example 2

### Isopropyl 9-syn-({6-[5-(methylsulfonyl)-2,3-dihydro-1H-indol-1-yl]pyrimidin-4-yl}oxy)-3-oxa-7-azabicyclo[3.3.1]nonane-7-carboxylate

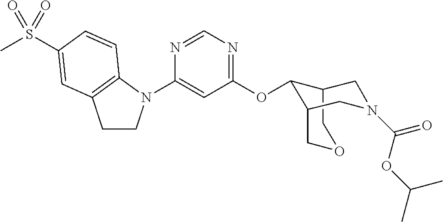

This compound was prepared from isopropyl 9-syn-hydroxy-3-oxa-7-azabicyclo[3.3.1]nonane-7-carboxylate and 1-(6-chloropyrimidin-4-yl)-5-(methylsulfonyl)indoline in a manner similar to that described for Example 1. The crude product was purified via column chromatography (0-10% acetone in dichloromethane) to give isopropyl 9-syn-({6-[5-(methylsulfonyl)-2,3-dihydro-1H-indol-1-yl]pyrimidin-4-yl}oxy)-3-oxa-7-azabicyclo[3.3.1]nonane-7-carboxylate as a white solid (24% yield). ¹H NMR (400 MHz, deuterochloroform) delta 1.27 (d, J=6.1 Hz, 6H), 1.96 (d, J=18.0 Hz, 2 H), 3.05 (s, 3H), 3.24 (d, J=13.7 Hz, 1H), 3.33 (m, 3H), 3.85 (d, J=11.2 Hz, 1H), 3.92 (d, J=11.4 Hz, 1H), 4.08-4.12 (m, 4H), 4.47 (d, J=13.9 Hz, 1H), 4.63 (d, J=13.4 Hz, 1H), 4.98 (m, 1H), 5.36 (br. s., 1H), 6.08 (s, 1H), 7.73 (s, 1H), 7.80 (d, J=8.4 Hz, 1H), 8.51 (s, 1H), 8.59 (d, J=8.4 Hz, 1H); LCMS (ES+) 503.2 (M+1).

### Example 3

### Isopropyl 4-({6-[5-(methylsulfonyl)-2,3-dihydro-1H-indol-1-yl]pyrimidin-4-yl}oxy)piperidine-1-carboxylate

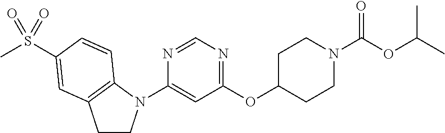

This compound was prepared from isopropyl 4-hydroxypiperidine-1-carboxylate and 1-(6-chloropyrimidin-4-yl)-5-(methylsulfonyl)indoline in a manner similar to that described for Example 1. This compound was purified by column chromatography (1:1 dichloromethane in acetone) to give a dark tan solid which was further purified via heating in methyl ethyl ketone. Upon cooling to room temperature, the mixture was diluted with methyl tert-butyl ether followed by filtration. The collected material was washed with methyl tert-butylether and then dried under vacuum to give isopropyl 4-({6-[5-(methylsulfonyl)-2,3-dihydro-1H-indol-1-yl]pyrimidin-4-yl}oxy)piperidine-1-carboxylate (7.89 g, 60%) as a white solid. ¹H NMR (500 MHz, deuterochloroform) delta 1.27 (d, J=6.1 Hz, 6H), 1.70-1.80 (m, 2H), 1.97-2.07 (m, 2H), 3.05 (s, 3H), 3.28-3.38 (m, 2 H), 3.33 (d, J=8.8 Hz, 2H), 3.78-3.89 (m, 2H), 4.07 (t, J=8.7 Hz, 2H), 4.88-4.98 (m, 1H), 5.34 (dd, J=8.0, 3.9 Hz, 1H), 5.99 (s, 1H), 7.73 (s, 1H), 7.79 (dd, J=8.5, 1.7 Hz, 1 H), 8.52 (s, 1H), 8.58 (d, J=8.5 Hz, 1H); LCMS (ES+): 461 (M+1).

### Example 4

### Isopropyl 9-syn-({5-cyano-6-[5-(methylsulfonyl)-1H-indol-1-yl]pyrimidin-4-yl}oxy)-3-oxa-7-azabicyclo[3.3.1]nonane-7-carboxylate

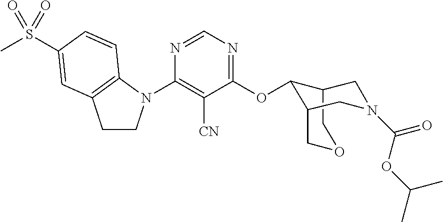

### Step A: Isopropyl 9-syn-({5-cyano-6-[5-(methylthio)-1H-indol-1-yl]pyrimidin-4-yl}oxy)-3-oxa-7-azabicyclo[3.3.1]nonane-7-carboxylate

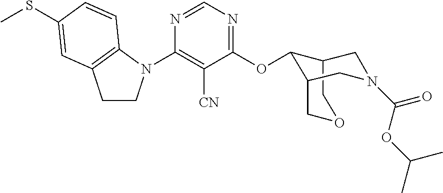

Isopropyl 9-syn-hydroxy-3-oxa-7-azabicyclo[3.3.1]nonane-7-carboxylate (95 mg, 0.41 mmol) in tetrahydrofuran (1 mL) was treated with a 1 M solution sodium bis(trimethylsilyl)amide in tetrahydrofuran (0.69 mL, 0.41 mmol). The reaction was stirred for 30 minutes, and then added drop-wise to a solution of 4-chloro-6-[5-(methylthio)-2,3-dihydro-1H-indol-1-yl]pyrimidine-5-carbonitrile (50 mg, 0.16 mmol) in tetrahydrofuran (1.5 mL). The resulting mixture was stirred at 70 degrees Celsius for 30 minutes. The reaction mixture was allowed to cool to room temperature and then quenched by the addition of saturated aqueous ammonium chloride. The reaction mixture was diluted with dichloromethane and water. The organic layer was separated, washed sequentially with saturated aqueous sodium bicarbonate and brine, and then dried over sodium sulfate. The mixture was filtered, and the filtrate was concentrated under reduced pressure. The crude product was purified by column chromatography (20-100% ethyl acetate in heptane) to give isopropyl 9-syn-({5-cyano-6-[5-(methylthio)-1H-indol-1-yl]pyrimidin-4-yl}oxy)-3-oxa-7-azabicyclo[3.3.1]nonane-7-carboxylate as a white solid (50 mg, 61%). ¹H NMR (400 MHz, deuterochloroform) delta 8.38 (s, 1H,), 8.14 (d, 1H, J=8.6 Hz), 7.17 (d, 1H, J=1.9 Hz), 7.11-7.16 (m, 1H), 5.47 (t, 1H, J=3.7 Hz), 4.91-5.01 (m, 1H,), 4.54 (dd, 2H, J=8.6, 7.9 Hz), 4.35 (d, 1H, J=14 Hz), 4.21 (br. s., 1H), 4.18 (s, 1H), 4.10-4.15 (m, 1H), 3.78-3.87 (m, 2H), 3.53-3.61 (m, 1H), 3.24 (t, 2H, J=8.2 Hz), 2.47 (s, 3H), 2.07 (br. s., 1H), 1.98 (1 br. s., 1H), 1.25 (d, 6H, J=6.8 Hz). LCMS (ES+)=517.8 (M+Na).

### Step B: 9-syn-({5-cyano-6-[5-(methylsulfonyl)-1H-indol-1-yl]pyrimidin-4-yl}oxy)-3-oxa-7-azabicyclo[3.3.1]nonane-7-carboxylate

To a solution isopropyl 9-syn-({5-cyano-6-[5-(methylthio)-1H-indol-1-yl]pyrimidin-4-yl}oxy)-3-oxa-7-azabicyclo[3.3.1]-nonane-7-carboxylate (50 mg, 0.10 mmol) in dichloromethane (10 mL) was added meta-chloroperoxybenzoic acid (67 mg, 0.27 mmol) in one portion. After 1 hour, the reaction was quenched with 3 drops of dimethyl sulfide. The reaction mixture was diluted with dichloromethane and washed with 0.5 M aqueous sodium hydroxide solution. The organic layer was separated and dried over sodium sulfate. The mixture was filtered, and filtrate was concentrated in vacuo to give a white powder which was purified by column chromatography (20-90% ethyl acetate in heptane) to give 9-syn-({5-cyano-6-[5-(methylsulfonyl)-1H-indol-1-yl]pyrimidin-4-yl}oxy)-3-oxa-7-azabicyclo[3.3.1]nonane-7-carboxylate as a yellow solid (35 mg, 66%). A portion of this material was further purified by dissolving a sample of the impure material in dichloromethane, and precipitating the product as a white solid by addition of heptane. ¹H NMR (400 MHz, deuterochloroform) delta 8.47 (s, 1H), 8.32 (d, 1H, J=8.6 Hz), 7.72-7.86 (m, 2H), 5.45 (t, 1H, J=3.5 Hz), 4.87-5.05 (m, 1H), 4.56-4.72 (m, 3H), 4.48 (d, 1H, J=14 Hz), 4.07-4.27 (m, 2H), 3.93 (d, 1H, J=12 Hz), 3.86 (d, 1H, J=12 Hz), 3.15-3.41 (m, 4H), 3.04 (s, 3H), 2.00 (br. s., 1H), 1.94 (br. s., 1H), 1.25 (d, 6H, J=7.0 Hz): LCMS (ES+)=528.0 (M+1).

### Example 5

### Isopropyl 9-anti-({5-cyano-6-[5-(methylsulfonyl)-1H-indol-1-yl]pyrimidin-4-yl}oxy)-3-oxa-7-azabicyclo[3.3.1]nonane-7-carboxylate

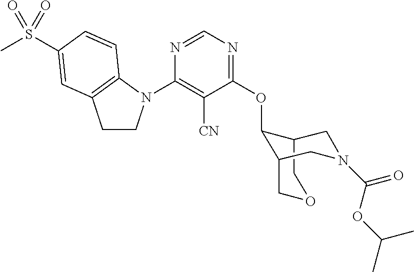

This compound was prepared in a two step procedure similar to that for the preparation of 9-syn-({5-cyano-6-[5-(methylsulfonyl)-1H-indol-1-yl]pyrimidin-4-yl}oxy)-3-oxa-7-azabicyclo[3.3.1]nonane-7-carboxylate. In the first step, 9-anti-hydroxy-3-oxa-7-azabicyclo[3.3.1]nonane-7-carboxylate was combined with 4-chloro-6-[5-(methylthio)-2,3-dihydro-1H-indol-1-yl]pyrimidine-5-carbonitrile to provide isopropyl 9-anti-({5-cyano-6-[5-(methylthio)-1H-indol-1-yl]pyrimidin-4-yl}oxy)-3-oxa-7-azabicyclo[3.3.1]nonane-7-carboxylate. In the second step, this intermediate was oxidized to afford isopropyl 9-anti-({5-cyano-6-[5-(methylsulfonyl)-1H-indol-1-yl]pyrimidin-4-yl}oxy)-3-oxa-7-azabicyclo[3.3.1]nonane-7-carboxylate which was purified by column chromatography (30%-100% ethyl acetate in heptane) to the product as a white solid (45 mg, 84%). ¹H NMR (400 MHz, deuterochloroform) delta 8.47 (s, 1H) 8.27-8.38 (m, 1H) 7.72-7.88 (m, 2H) 5.51 (t, 1H, J=3.6 Hz) 4.89-5.02 (m, 1H) 4.63 (t, 2H, J=8.5 Hz) 4.37 (d, 1H, J=14 Hz) 4.17-4.28 (m, 2H) 4.14 (d, 1H, J=11 Hz) 3.77-3.88 (m, 2H) 3.51-3.63 (m, 1H) 3.39-3.49 (m, 1H) 3.34 (t, 2H, J=8.4 Hz) 3.04 (s, 3H) 2.07 (br. s., 1H) 2.00 (br. s., 1H) 1.25 (d, 6H, J=6.2 Hz); LCMS (ES+)=528.0 (M+1).

### Example 6

### Isopropyl 4-({5-cyano-6-[5-(methylsulfonyl)-2,3-dihydro-1H-indol-1-yl]pyrimidin-4-yl}oxy)piperidine-1-carboxylate

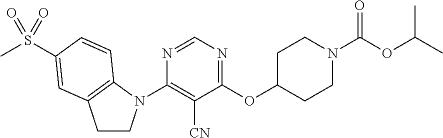

This compound was prepared in two step procedure similar to that used for the preparation of 9-syn-({5-cyano-6-[5-(methylsulfonyl)-1H-indol-1-yl]pyrimidin-4-yl}oxy)-3-oxa-7-azabicyclo[3.3.1]nonane-7-carboxylate. In the first step, isopropyl 4-hydroxypiperidine-1-carboxylate was combined with 4-chloro-6-[5-(methylthio)-2,3-dihydro-1H-indol-1-yl]pyrimidine-5-carbonitrile to yield isopropyl 4-({5-cyano-6-[5-(methylthio)-2,3-dihydro-1H-indol-1-yl]pyrimidin-4-yl}oxy)piperidine-1-carboxylate. In the second step, this intermediate was oxidized to afford 4-({5-cyano-6-[5-(methylsulfonyl)-2,3-dihydro-1H-indol-1-yl]pyrimidin-4-yl}oxy)piperidine-1-carboxylate, which was purified by column chromatography (20-90% ethyl acetate in heptane) to give isopropyl 4-({5-cyano-6-[5-(methylsulfonyl)-2,3-dihydro-1H-indol-1-yl]pyrimidin-4-yl}oxy)piperidine-1-carboxylate_as a solid. This compound was further purified by precipitation from a solution of dichloromethane and heptane to give a yellow solid (30 mg, 82%). ¹H NMR (400 MHz, deuterochloroform) delta 8.46 (s, 1H), 8.29 (d, J=8.60 Hz, 1H), 7.73-7.85 (m, 2H), 5.40-5.54 (m, 1H), 4.86-5.00 (m, 1H), 4.61 (t, J=8.40 Hz, 2H), 3.66-3.82 (m, 2H), 3.40-3.53 (m, 2H), 3.33 (t, J=8.40 Hz, 2H), 3.04 (s, 3H), 1.92-2.09 (m, 2 H), 1.76-1.90 (m, 2H), 1.25 (d, J=6.25 Hz, 6H); LCMS (ES+): 486.3 (M+1).

### Example 7

### Isopropyl 4-({5-methoxy-6-[5-(methylsulfonyl)-2,3-dihydro-1H-indol-1-yl]pyrimidin-4-yl}oxy)piperidine-1-carboxylate

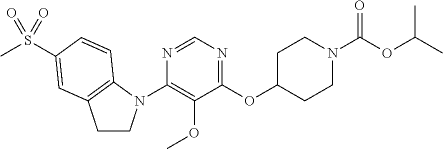

### Step A: Isopropyl 4-({5-methoxy-6-[5-(methylthio)-2,3-dihydro-1H-indol-1-yl]pyrimidin-4-yl}oxy)piperidine-1-carboxylate

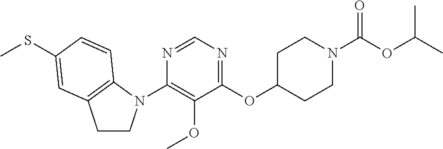

A vial sealed with a cap containing a Teflon septa was charged with Pd₂(dba)₃ (43 mg, 0.046 mmol), 2-dicyclohexylphosphino-2′,4′,6′-triisopropylbiphenyl (33 mg, 0.068 mmol), sodium tert-butoxide (21 mg, 0.213 mmol), isopropyl 4-[(6-chloro-5-methoxypyrimidin-4-yl)oxy]piperidine-1-carboxylate (50 mg, 0.15 mmol) and 5-(methylthio)indoline (30 mg 0.182 mmol). Degassed (purged of oxygen) toluene (2 mL) was added, and the resulting mixture was heated at 120 degrees Celsius for 12 hours. The resulting mixture was diluted with ethyl acetate (30 mL), washed sequentially with water, saturated sodium bicarbonate and brine. The organic solution was then dried over sodium sulfate, filtered, and the filtrate was concentrated under reduced pressure. The crude material was purified by column chromatography (10-90% ethyl acetate in heptane) to give impure isopropyl 4-({5-methoxy-6-[5-(methylthio)-2,3-dihydro-1H-indol-1-yl]pyrimidin-4-yl}oxy)piperidine-1-carboxylate as a solid (55 mg, 75%) which was used without further purification in the next step. LCMS (ES+): 459.0 (M+1).

### Step B: Isopropyl 4-({5-methoxy-6-[5-(methylsulfonyl)-2,3-dihydro-1H-indol-1-yl]pyrimidin-4-yl}oxy)piperidine-1-carboxylate

This compound was prepared in a similar manner to the preparation of 9-syn-({5-cyano-6-[5-(methylsulfonyl)-1H-indol-1-yl]pyrimidin-4-yl}oxy)-3-oxa-7-azabicyclo[3.3.1]nonane-7-carboxylate (Example 4, step B) using isopropyl 4-({5-methoxy-6-[5-(methylthio)-2,3-dihydro-1H-indol-1-yl]pyrimidin-4-yl}oxy)piperidine-1-carboxylate as starting material. The crude material was purified by column chromatography (20-90% ethyl acetate in heptane) and subsequent precipitation from a solution of ethyl acetate and heptane to give isopropyl 4-({5-methoxy-6-[5-(methylsulfonyl)-2,3-dihydro-1H-indol-1-yl]pyrimidin-4-yl}oxy)piperidine-1-carboxylate as a white solid (25 mg, 42%). ¹H NMR (400 MHz, deuterochloroform) delta 8.21 (1H, s), 7.65-7.73 (3H, m), 5.31-5.42 (1H, m), 4.86-4.97 (1H, m), 4.32 (2H, t, J=8.59 Hz), 3.77-3.86 (2H, m), 3.75 (3H, s), 3.33-3.44 (2H, m), 3.22 (2H, t, J=8.59 Hz), 3.01 (3 H, s), 1.97-2.11 (2H, m), 1.73-1.91 (2H, m), 1.25 (6H, d, J=6.25 Hz); LCMS (ES+): 491.2 (M+1).

### Example 8

### Isopropyl 4-({5-methyl-6-[5-(methylsulfonv1)-2,3-dihydro-1H-indol-1-yl]pyrimidin-4-yl}oxy)piperidine-1-carboxylate

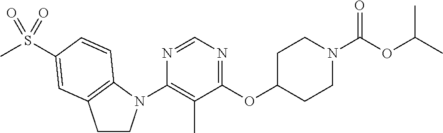

This compound was prepared in two step procedure similar to that used for the preparation of isopropyl 4-({5-methoxy-6-[5-(methylsulfonyl)-2,3-dihydro-1H-indol-1-yl]pyrimidin-4-yl}oxy)piperidine-1-carboxylate (Example 7). In the first step, isopropyl 4-[(6-chloro-5-methylpyrimidin-4-yl)oxy]piperidine-1-carboxylate was combined with 5-(methylthio)indoline to yield isopropyl 4-({5-methyl-6-[5-(methylthio)-2,3-dihydro-1H-indol-1-yl]pyrimidin-4-yl}oxy)piperidine-1-carboxylate. In the second step, this intermediate was oxidized to afford isopropyl 4-({5-methyl-6-[5-(methylsulfonyl)-2,3-dihydro-1H-indol-1-yl]pyrimidin-4-yl}oxy)piperidine-1-carboxylate, which was purified by column chromatography (50-70% ethyl acetate in heptane) to provide an off-white solid (88 mg, 81%). ¹H NMR (400 MHz, deuterochloroform) delta 1.22 (d, J=6.3 Hz, 6 H) 1.71-1.85 (m, 2H) 2.01 (br. s., 5H) 2.98 (s, 3H) 3.17 (t, J=8.3 Hz, 2H) 3.39 (br. s., 2H) 3.73 (br. s., 2H) 4.15 (t, J=8.3 Hz, 2H) 4.84-4.97 (m, 1H) 5.30-5.41 (m, 1H) 6.66 (d, J=8.2 Hz, 1H) 7.63 (d, J=8.4 Hz, 1H) 7.66 (s, 1H) 8.38 (s, 1H). LCMS (ES+): 475.4 (M+1).

### Example 9

### 4-[6-(5-Dimethylcarbamoyl-2,3-dihydroindol-1-yl)-pyrimidin-4-yloxy]-piperidine-1-carboxylic acid isopropyl ester

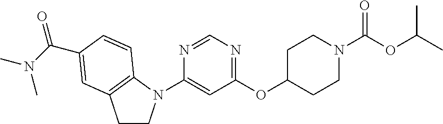

### Step A: 1-[6-(1-Isopropoxycarbonyl-piperidin-4-yloxy)-pyrimidin-4-yl]-2,3-dihydro-1H-indole-5-carboxylic acid

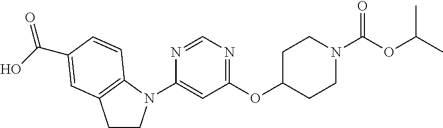

A mixture of 1-(6-chloro-pyrimidin-4-yl)-2,3-dihydro-1H-indole-5-carboxylic acid (62.0 mg, 0.225 mmol) and 4-hydroxypiperidine-1-carboxylic acid isopropyl ester (54.9 mg, 0.293 mmol) in anhydrous 1,4-dioxane (2.0 mL) was heated to 105 degrees Celsius. After stirring for 5 minutes, a 1 M solution of sodium bis(trimethylsilyl)amide in tetrahydrofuran (0.54 mL, 0.54 mmol) was added. After 2 hours, the reaction mixture was diluted with water and concentrated under reduced pressure. The resulting residue was taken up in dichloromethane and washed with saturated aqueous sodium bicarbonate. The aqueous phase was extracted three times with dichloromethane, and the combined organic layers were dried over sodium sulfate and filtered. The filtrate was concentrated under reduced pressure, and the crude residue was purified by column chromatography (20-70% ethyl acetate in heptane) to afford 146-(1-isopropoxycarbonyl-piperidin-4-yloxy)-pyrimidin-4-yl]-2,3-dihydro-1H-indole-5-carboxylic acid (30 mg, 31%) as a white foam. ¹H NMR (400 MHz, deuterochloroform) delta 1.24 (d, J=6.25 Hz, 6H) 1.67-1.79 (m, 2H) 1.93-2.05 (m, 2H) 3.22-3.36 (m, 4H) 3.75-3.87 (m, 2H) 4.03 (t, J=8.69 Hz, 2H) 4.87-4.97 (m, 1H) 5.27-5.35 (m, 1H) 5.97 (s, 1 H) 7.89 (s, 1H) 7.98 (d, J=10.15 Hz, 1H) 8.43 (d, J=8.00 Hz, 1H) 8.49 (s, 1H)

### Step B: 4-[6-(5-Dimethylcarbamoyl-2,3-dihydroindol-1-yl)-pyrimidin-4-yloxy]-piperidine-1-carboxylic acid isopropyl ester

To a mixture of the carboxylic acid (30 mg, 0.07 mmol), diisopropylethylamine (0.024 mL, 0.14 mmol), and O-benzotriazole-N,N,N′, N′-tetramethyl-uronium-hexafluoro-phosphate (35 mg, 0.091 mmol) in N,N-dimethylformamide (1.0 mL) was added a 2 M solution of dimethylamine in tetrahydrofuran (0.052 mL, 0.105 mmol). After 2 hours, the reaction mixture was diluted with water and extracted three times with ethyl acetate. The combined organic layers were dried over sodium sulfate, filtered, and the filtrate concentrated under reduced pressure. The crude residue was purified by column chromatography (20-70% ethyl acetate in heptane) to afford 4-[6-(5-dimethylcarbamoyl-2,3-dihydroindol-1-yl)-pyrimidin-4-yloxy]-piperidine-1-carboxylic acid isopropyl ester (8 mg, 30%) as a white solid. ¹H NMR (400 MHz, deuterochloroform) delta 1.25 (d, J=6.05 Hz, 6H) 1.68-1.78 (m, 2H) 1.95-2.04 (m, 2H) 3.06 (br. s., 6H) 3.24 (t, J=8.69 Hz, 2H) 3.28-3.36 (m, 2H) 3.81 (broad s., 2H) 3.99 (t, J=8.59 Hz, 2H) 4.89-4.96 (m, 1H) 5.27-5.34 (m, 1H) 5.93 (s, 1H) 7.29 (d, 1H) 7.32 (s, 1H) 8.36 (d, J=8.40 Hz, 1H) 8.46 (s, 1H). LCMS (ES+): 454.4 (M+1).

### Example 10

### Isopropyl 4-{[6-(5-cyano-2,3-dihydro-1H-indol-1-yl)pyrimidin-4-yl]oxy}piperidine-1-carboxylate

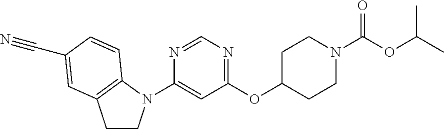

A mixture of 4-(6-chloro-pyrimidin-4-yloxy)-piperidine-1-carboxylic acid isopropyl ester (50.4 mg, 0.168 mmol) and 2,3-dihydro-1H-indole-5-carbonitrile (22.0 mg, 0.15 mmol) in anhydrous 1,4-dioxane (2.0 mL) was heated to 105 degrees Celsius. After stirring for 5 min, a 1 M solution of sodium bis(trimethylsilyl)amide in tetrahydrofuran (0.184 mL, 0.184 mmol) was added. After 30 minutes, the reaction mixture was quenched with saturated aqueous ammonium chloride and concentrated under reduced pressure. The resulting residue was dissolved in dichloromethane and washed with saturated aqueous sodium bicarbonate. The aqueous phase was extracted three times with dichloromethane, and the combined organic layers were dried over sodium sulfate and filtered. The filtrate was concentrated under reduced pressure, and the crude residue was purified by column chromatography (20-60% ethyl acetate in heptane) to afford isopropyl 4-{[6-(5-cyano-2,3-dihydro-1H-indol-1-yl)pyrimidin-4-yl]oxy}piperidine-1-carboxylate (38 mg, 61%) as a white solid. ¹H NMR (400 MHz, deuterochloroform) delta 1.25 (d, J=6.25 Hz, 6H) 1.68-1.78 (m, 2H) 1.96-2.04 (m, 2H) 3.24-3.36 (m, 4 H) 3.78-3.87 (m, 2H) 4.03 (t, J=8.79 Hz, 2H) 4.89-4.97 (m, 1H) 5.29-5.36 (m, 1H) 5.96 (s, 1H) 7.43 (s, 1H) 7.51 (dd, J=8.79, 1.17 Hz, 1H) 8.48-8.53 (m, 2H). LCMS (ES+): 408.4 (M+1).

### Example 11

### Isopropyl 4-[(6-{5-[(2-hydroxyethyl)sulfonyl]-2,3-dihydro-1H-indol-1-yl}pyrimidin-4-yl)oxy]piperidine-1-carboxylate

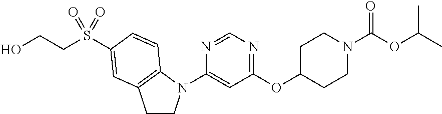

### Step A: Isopropyl 4-[(6-{5-[(2-{]tert-butyl(dimethyl)silyl]oxy}ethyl)thio]-2,3-dihydro-1H-indol-1-yl}pyrimidin-4-yl)oxy]piperidine-1-carboxylate

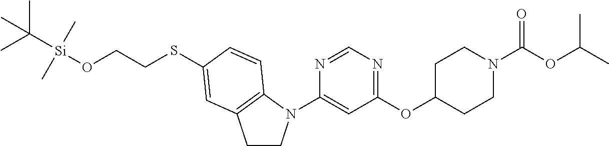

Compounds 5-[(2-{[tert-butyl(dimethyl)silyl]oxy}ethyl)thio]indoline (143 mg, 0.462 mmol) and isopropyl 4-[(6-chloro-pyrimidin-4-yl)oxy]piperidine-1-carboxylate (146.9 mg, 0.49 mmol) were combined in anhydrous 1,4-dioxane (2.0 mL) in a vial with a Teflon septa. The solution was heated to 100 degrees Celsius for 5 minutes, and then a 1 M solution of sodium bis(trimethylsilyl)amide in tetrahydrofuran (0.55 mL, 0.55 mmol) was added. The mixture was stirred for 1 hour at 100 degrees Celsius. The reaction was then allowed to cool to room temperature, concentrated under reduced pressure, and the residue was diluted with ethyl acetate (40 mL). The solution was then washed twice with saturated sodium bicarbonate (25 mL) and dried over magnesium sulfate. The mixture was filtered, and the filtrate was concentrated under reduced pressure to give a brown oil that was purified by column chromatography to afford isopropyl 4-[(6-{5-[(2-{[tert-butyl(dimethyl)silyl]oxy}ethyl)thio]-2,3-dihydro-1H-indol-1-yl}pyrimidin-4-yl)oxy]piperidine-1-carboxylate (164.1 mg, 62%). ¹H NMR (400 MHz, deuterochloroform) delta 0.02 (s, 6H) 0.86 (s, 9H) 1.24 (d, 6H) 1.65-1.77 (m, 2H) 1.92-2.04 (m, 2H) 2.94-2.99 (m, 2H) 3.19 (t, J=8.59 Hz, 2H) 3.26-3.35 (m, 2H) 3.71-3.77 (m, 2H) 3.77-3.88 (m, 2H) 3.95 (t, J=8.59 Hz, 2H) 4.87-4.96 (m, 1H) 5.27-5.29 (m, 1H) 5.89 (d, J=0.98 Hz, 1H) 7.24-7.25 (m, 2H) 8.25 (d, J=8.98 Hz, 1 H) 8.43 (d, J=0.78 Hz, 1H).

### Step B: Isopropyl 4-[(6-{5-[(2-{[tert-butyl(dimethyl)silyl]oxy}ethyl)sulfonyl]-2,3-dihydro-1H-indol-1-yl}pyrimidin-4-yl)oxy]piperidine-1-carboxylate

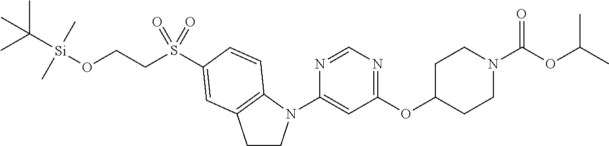

Isopropyl 4-[(6-{5-[(2-{[tert-butyl(dimethyl)silyl]oxy}ethyl)thio]-2,3-dihydro-1H-indol-1-yl}pyrimidin-4-yl)oxy]piperidine-1-carboxylate (80 mg, 0.14 mmol) was dissolved in chloroform (3.0 mL) and cooled to −5 degrees Celsius. meta-chloroperbenzoic acid (81.6 mg, 0.36 mmol) was added in one portion, and the reaction allowed to slowly warm to room temperature over 45 minutes. The reaction mixture was then concentrated under reduced pressure, and the crude residue was purified by column chromatography to afford isopropyl 4-[(6-{5-[(2-{[tert-butyl(dimethyl)silyl]oxy}ethyl)sulfonyl]-2,3-dihydro-1H-indol-1-yl}pyrimidin-4-yl)oxy]piperidine-1-carboxylate (78 mg, 92%) as a clear oil. ¹H NMR (400 MHz, deuterochloroform) delta 0.03 (s, 6H) 0.77 (s, 9H) 1.23 (s, 3H) 1.25 (s, 3H) 1.66-1.79 (m, 2H) 1.97 (br. s., 2H) 3.24-3.30 (m, 2H) 3.29-3.35 (m, 4H) 3.75-3.87 (m, 2 H) 3.96 (t, J=6.54 Hz, 2H) 4.03 (t, J=8.78 Hz, 2H) 4.86-4.97 (m, 1H) 5.27-5.35 (m, 1 H) 5.96 (d, J=0.78 Hz, 1H) 7.66 (d, J=1.56 Hz, 1H) 7.72 (dd, J=8.69, 2.05 Hz, 1H) 8.49 (d, J=0.78 Hz, 1H) 8.53 (d, J=8.78 Hz, 1H).

### Step C: Isopropyl 4-[(6-{5-[(2-hydroxyethyl)sulfonyl)-2,3-dihydro-1H-indol-1-yl}pyrimidin-4-yl)oxy]piperidine-1-carboxylate

Isopropyl 4-[(6-{5-[(2-{[tert-butyl(dimethyl)silyl]oxy}ethyl)sulfonyl]-2,3-dihydro-1H-indol-1-yl}pyrimidin-4-yl)oxy]piperidine-1-carboxylate (75 mg, 0.12 mmol) was dissolved in 1,4-dioxane (3 mL) and a 4 M solution of hydrochloric acid in 1,4-dioxane (0.25 mL) was added. The reaction was stirred for 25 minutes, and then was filtered to obtain isopropyl 4-[(6-{5-[(2-hydroxyethyl)sulfonyl]-2,3-dihydro-1H-indol-1-yl]pyrimidin-4-yl)oxy}piperidine-1-carboxylate (21 mg, 35%) as a white solid. ¹H NMR (400 MHz, deuterochloroform) delta 1.24 (d, J=6.25 Hz, 6H) 1.70-1.82 (m, 2H) 2.05-2.14 (m, 2 H) 3.31-3.43 (m, 6H) 3.84-3.93 (m, 2H) 3.99-4.03 (m, 2H) 4.20-4.27 (m, 2H) 4.91 (q, 1H) 5.52-5.59 (m, 1H) 6.14 (s, 1H) 7.80 (s, 1H) 7.84 (d, J=8.59 Hz, 1H) 8.43 (d, J=8.39 Hz, 1H) 8.69 (s, 1H). LCMS (ES+): 491 (M+1).

### Example 12

### Isopropyl 4-{[6-(2,3-dihydro-1H-pyrrolo[3,2-b]pyridin-1-yl))pyrimidin-4-yl]oxy}piperidine-1-carboxylate

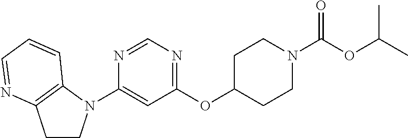

To a solution of isopropyl 4-hydroxypiperidine carboxylate (80.5 mg, 0.43 mmol) and anhydrous N,N-dimethylformamide (5 mL) was added sodium hydride (19 mg, 0.47 mmol), and the mixture was stirred for 20 minutes. 1-(6-Chloropyrimidin-4-yl)-2,3-dihydro-1H-pyrrolo[3,2-b]pyridine (100 mg, 0.43 mmol) was added, and the reaction was heated at 60 degrees Celsius for 14 hours. The reaction was diluted with methyl tert-butyl ether and washed with water. The phases were separated, and the aqueous layer was extracted sequentially with methyl tert-butyl ether and ethyl acetate. The combined organic extracts were washed with water followed by brine and then dried over sodium sulfate. The mixture was filtered, and the filtrate concentrated under reduced pressure. The residue was purified by column chromatography (5% methanol/0.5% triethylamine in ethyl acetate) to give isopropyl 4-{[6-(2,3-dihydro-1H-pyrrolo[3,2-b]pyridin-1-yl)pyrimidin-4-yl]oxy}piperidine-1-carboxylate as a thick oil (90 mg, 55%). ¹H NMR (400 MHz, deuterochloroform): delta 8.63 (d, J=8.3 Hz, 0.5H), 8.57 (d, J=8.3 Hz, 0.5H), 8.60 (s, 0.5H), 8.45 (s, 0.5H), 8.16 (d, J=4.98 Hz, 0.5H), 8.07 (d, J=4.98 Hz, 0.5H), 7.10-7.15 (m, 1H), 6.62 (s, 0.5H), 5.92 (s, 0.5H), 4.87-4.94 (m, 2H), 4.00-4.13 (m, 2H), 3.81-3.98 (m, 2H), 3.30-3.4 (m, 2H), 3.03-3.11 (m, 2H), 1.83-2.03 (m, 2H), 1.41-1.51 (m, 2H), 1.17-1.26 (m, 6H). LCMS (ES+): 384 (M+1).

### Example 13

### Isopropyl 4-{[6-(2,3-dihydro-1H-pyrrolo[3,2-b]pyridin-1-yl)-5-methylpyrimidin-4-yl]oxy}-piperidine-1-carboxylate

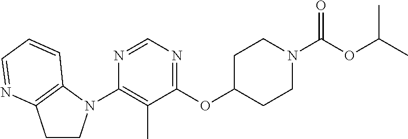

This compound was prepared from 1-(6-chloro-5-methylpyrimidin-4-yl)-2,3-dihydro-1H-pyrrolo[3,2-b]pyridine and isopropyl 4-hydroxypiperidine carboxylate in a manner similar to that described for the preparation of isopropyl 4-{[6-(2,3-dihydro-1H-pyrrolo[3,2-b]pyridin-1-yl)pyrimidin-4-yl]oxy}piperidine-1-carboxylate (Example 12). The crude product was dissolved in diethyl ether. Heptane was slowly added causing the pure isopropyl-4-{[6-(2,3-dihydro-1H-pyrrolo[3,2-b]pyridin-1-yl)-5-methylpyrimidin-4-yl]oxy}-piperidine-1-carboxylate (75 mg, 85%) to come out of solution as an oil. ¹H NMR (400 MHz, deuterochloroform): delta 8.37 (s, 1H), 8.00 (d, J=5.8 Hz, 1H), 6.97-7.04 (m, 2H), 4.87-4.96 (m, 2H), 4.14 (t, 1H), 3.72-3.88 (m, 3H), 3.37-3.42 (m, 1H), 3.28 (t, 1H), 3.03-3.09 (m, 2H), 2.05 (s, 3H), 1.74-1.87 (m, 2H), 1.42-1.50 (m, 2 H),1.02-1.26 (m. 6H). LCMS (ES+): 398 (M+1).

### Example 14

### Isopropyl 4-({6-[5-(methylsulfonyl)-2,3-dihydro-1H-pyrrolo[2,3-b]pyridin-1-yl]pyrimidin-4-yl}oxy)piperidine-1-carboxylate

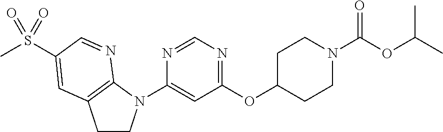

This compound was prepared from isopropyl 4-hydroxypiperidine-1-carboxylate and 1-(6-chloropyrimidin-4-yl)-5-(methylsulfonyl)-2,3-dihydro-1H-pyrrolo[2,3-b]pyridine in a manner similar to that described for Example 1. The crude product was purified via column chromatography (ethyl acetate in heptane gradient) to give a white solid which was heated in acetonitrile and allowed to cool. The mixture was filtered, and the isolated white solid was dried under vacuum to give 4-({6-[5-(methylsulfonyl)-2,3-dihydro-1H-pyrrolo[2,3-b]pyridin-1-yl]pyrimidin-4-yl}oxy)piperidine-1-carboxylate (282 mg, 52% yield). ¹H NMR (400 MHz, deuterchloroform): delta 1.27 (d, J=6.2 Hz, 6H), 1.73-1.84 (m, 2H), 1.96-2.06 (m, 2H), 3.09 (s, 3H), 3.24 (t, J=8.7 Hz, 2H), 3.31-3.41 (m, 2H), 3.78-3.89 (m, 2H), 4.44 (dd, J=9.4, 8.1 Hz, 2H), 4.90-4.99 (m, 1H), 5.32 (tt, J=7.9, 3.8 Hz, 1H), 7.84 (dt, J=2.4, 1.3 Hz, 1H), 8.12 (d, J=1.0 Hz, 1H), 8.53 (d, J=1.0 Hz, 1 H), 8.70 (dt, J=2.1, 0.7 Hz, 1H). LCMS (ES+): 462 (M+1).

### Example 15

### tert-Butyl 3-fluoro-4-({6-[5-(methylsulfonyl)-2,3-dihydro-1H-indol-1-yl]pyrimidin-4-yl}oxy)piperidine-1-carboxylate

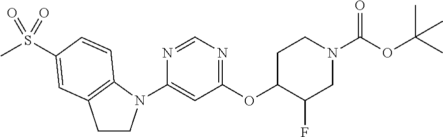

To a hot (105 degrees Celsius) solution of 1-(6-chloropyrimidin-4-yl)-5-(methylsulfonyl)indoline (33.8 mg, 0.109 mmol) and tert-butyl-3-fluoro-4-hydroxypiperidine-1-carboxylate (racemic mixture of cis and trans isomers) (20 mg, 0.091 mmol) in 1.5 mL of 1,4-dioxane, in a microwave vial, was added sodium bis(trimethylsilyl)amide (0.14 mL, 1 M in tetrahydrofuran). The stirred mixture was heated at 105 degrees Celsius under a nitrogen atmosphere for 4 hours before it was cooled to room temperature and diluted with water and ethyl acetate. The organic phase was removed and washed with saturated aqueous sodium bicarbonate. The combined aqueous phases were extracted with ethyl acetate. The combined organic layers were dried over magnesium sulphate, filtered, and the filtrate was concentrated in vacuo. The residue was dissolved in dimethyl sulfoxide (1 mL) and purified by reversed-phase HPLC (Column: Waters XBridge C₁₈19×100 mm, 5 micrometer; Mobile phase A: 0.03% ammonium hyroxide in water (v/v); Mobile phase B: 0.03% ammonium hydroxide in acetonitrile (v/v); Gradient: 80% water/20% acetonitrile linear to 0% water/100% acetonitrile in 8.5 minutes, hold at 0% water/100% acetonitrile to 10.0 minutes. Flow: 25 mL/min. Purification in this way provided 17 mg of tert-Butyl 3-fluoro-4-({645-(methylsulfonyl)-2,3-dihydro-1H-indol-1-yl]pyrimidin-4-yl}oxy)piperidine-1-carboxylate. LCMS (M+H)): 493.0

### Example 16

### tert-Butyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate (racemic)

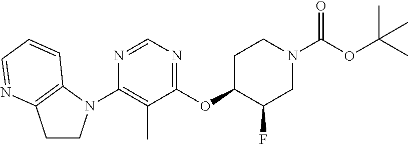

To a stirred solution of racemic (3R,4S)-tert-butyl 3-fluoro-4-hydroxypiperidine-1-carboxylate (31.3 mg, 0.081 mmol) in 3 mL anhydrous N,N-dimethylformamide, was added sodium hydride (6.5 mg, 0.16 mmol) at room temperature. The mixture was stirred under nitrogen at room temperature for 20 minutes. 1-(6-chloro-5-methylpyrimidin-4-yl)-2,3-dihydro-1H-pyrrolo[3,2-b]pyridine (20 mg, 0.081 mmol) was added, and the reaction was heated to 60 degrees Celsius under nitrogen for 16 hours. The mixture was cooled to room temperature, diluted with water and extracted with ethyl acetate. The combined extracts were washed with water, brine, dried over sodium sulfate, filtered and the filtrate was concentrated in vacuo. The residue was purified by flash chromatography eluting with an isocratic mixture of 20-80% ethyl acetate and heptane to give a yellow gum (12 mg). This gum was dissolved in dimethyl sulfoxide (1 mL) and purified by reversed-phase HPLC Column: Waters Sunfire C₁₈19×100 mm, 5 micrometer; Mobile phase A: 0.05% trifluoroacetic acid in water (v/v); Mobile phase B: 0.05% trifluoroacetic acid in acetonitrile (v/v); Gradient: 95% water/5% acetonitrile linear to 0% water/100% acetonitrile in 8.5 minutes, hold at 0% water/100% acetonitrile to 10.0 minutes. Flow: 25 mL/min. LCMS (M+H): 430.2.

This example was also prepared as follows:

To a 3-necked round bottom flask was added 1-(6-chloro-5-methylpyrimidin-4-yl)-2,3-dihydro-1H-pyrrolo[3,2-b]pyridine (1.0 g 4.05 mmol), (3R,4S)-tert-butyl 3-fluoro-4-hydroxypiperidine-1-carboxylate (0.98 g, 4.47 mmol), cesium carbonate (1.58 g, 4.85 mmol) and acetonitrile (5 mL). The mixture was heated at refluxed for approximately 48 hours. Water (3 volumes) was added and the mixture was then concentrated in vacuo to remove the acetonitrile. The residue was diluted with ethyl acetate (10 volumes) and the layers were separated. The aqueous layer was extracted with ethyl acetate (1 volume). The combined organic layers were dried over magnesium sulfate, filtered and the filtrate was concentrated to give tert-butyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate (racemic) as a dark brown oil, (1.6 g crude, 92%)

This example was also prepared as follows:

To a three neck round bottom flask was added 1-(6-chloro-5-methylpyrimidin-4-yl)-2,3-dihydro-1H-pyrrolo[3,2-b]pyridine (19 g, 77.02 mmol) and 2-methyltetrahydrofuran (95 mL). The flask was purged with nitrogen and the mixture was heated at reflux. In a separate flask (3R,4S)-tert-butyl 3-fluoro-4-hydroxypiperidine-1-carboxylate (chiral) (18.6 g, 84.83 mmol) and 2-methyltetrahydrofuran (19 mL) were combined to make a thick slurry. Sodium bis(trimethylsilyl)amide (92.4 mL, 92.40 mmol) was added. The solution was stirred for several minutes and over time became orange in color. This resulting orange solution was added slowly drop-wise over 15 minutes to the hot (reflux) solution of 1-(6-chloro-5-methylpyrimidin-4-yl)-2,3-dihydro-1H-pyrrolo[3,2-b]pyridine. The resulting solution was heated at reflux for approximately 1.5 hours. The reaction was then cooled to room temperature and diluted with water (76 mL). The mixture was stirred overnight at room temperature. The layers were separated.

The aqueous layer was extracted with 2-methyltetrahydrofuran (40 mL). The combined organic layers were washed with cold, 0 degrees Celsius, 1 N hydrochloric acid (60 mL). The layers were separated. The orange aqueous layer was immediately adjusted to pH 9-10 with 1 N sodium hydroxide. This layer was extracted with 2-methyltetrahydrofuran (110 mL). This final organic extract was dried over magnesium sulfate, filtered and the filtrate was concentrated in vacuo to give (3R,4S)-tert-butyl 4-(6-(2,3-dihydro-1H-pyrrolo[3,2-b]pyridin-1-yl)-5-methylpyrimidin-4-yloxy)-3-fluoropiperidine-1-carboxylate as an orange oil (39 g, crude, 117.9%). This material was used without further purification in subsequent steps.

### Example 17

### 1-Methylcyclopropyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate

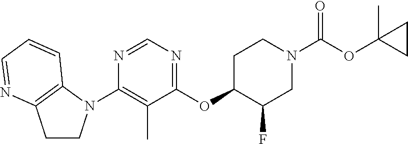

### Step A: 1-(64(3R,4S)-3-fluoropiperidin-4-yloxy)-5-methylpyrimidin-4-yl)-2,3-dihydro-1H-pyrrolo[3,2-b]pyridine

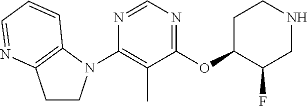

(3R,4S)-tert-butyl 4-(6-(2,3-dihydro-1H-pyrrolo[3,2-b]pyridin-1-yl)-5-methylpyrimidin-4-yloxy)-3-fluoropiperidine-1-carboxylate (31.7 g, 73.81 mmol) and p-toluenesulfonic acid monohydrate (56.16 g, 295.24 mmol) were combined in tetrahydrofuran (317 mL, 3.90 mole) and water (31 mL, 1.72 mole) The resulting solution was heated at reflux for 3 hours. The free-amine product was not isolated, and directly carried onto the next step.

### Step B: 1-methylcyclopropyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate

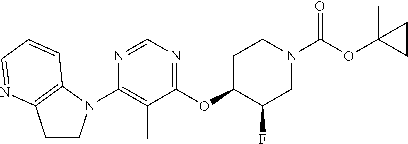

To the stirred mixture of 1-(6-((3R,4S)-3-fluoropiperidin-4-yloxy)-5-methylpyrimidin-4-yl)-2,3-dihydro-1H-pyrrolo[3,2-b]pyridine from Step A was added triethylamine (61.73 mL, 442.85 mmol) at room temperature. To this stirred solution (pH 9-10) was added 1-methylcyclopropyl 4-nitrophenyl carbonate (17.66 g, 73.81 mmol). The reaction mixture was stirred at 40 degrees Celsius for 3 hours. The mixture was then diluted with 1 N sodium hydroxide (3 volumes) and the solution was concentrated to remove tetrahydrofuran. The aqueous layer was extracted with 2-methyltetrahydrofuran (5 volumes). The combined organic extracts were washed twice with 1 N sodium hydroxide (2 volumes), saturated aqueous sodium carbonate (1 volume) and brine (1 volume), dried over magnesium sulfate, filtered and the filtrate was concentrated in vacuo to an oil. The oil was granulated with stirring in tert-butyl methyl ether for 16 hours. The yellow solids were collected by filtration. These solids were stirred as a suspension in 0.2N sodium hydroxide (2 volumes) for 2 hours. Filtration gave 19.8 g of 1-methylcyclopropyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate (63%). ¹H NMR (400 MHz, deuterochloroform) delta 0.56-0.68 (m, 2H) 0.83-0.93 (m, 2H) 1.54 (s, 3H) 1.83-2.22 (m, 5H) 3.04-3.49 (m, 4H) 3.96-4.29 (m, 4H) 4.74-4.99 (m, 1H) 5.30-5.46 (m, 1H) 6.98 (dd, J=8.10, 4.98 Hz, 1H) 7.03-7.11 (m, 1H) 8.00 (dd, J=4.88, 1.37 Hz, 1H) 8.33 (s, 1H).

### Example 18

### 1-Methylcyclopropyl (3S,4R)-4-{[6-(2,3-dihydro-1H-pyrrolo[3,2-b]pyridin-1-yl)-5-methylpyrimidin-4-yl]oxy}-3-fluoropiperidine-1-carboxylate

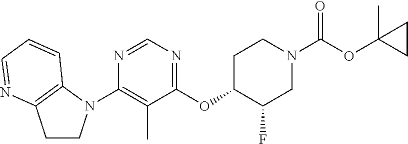

Example 18 was prepared in a manner analogous to Example 17 with appropriate starting materials.

¹H NMR (400 MHz, deuterochloroform) delta 0.56-0.68 (m, 2H) 0.83-0.93 (m, 2H) 1.54 (s, 3H) 1.83-2.22 (m, 5H) 3.04-3.49 (m, 4H) 3.96-4.29 (m, 4H) 4.74-4.99 (m, 1H) 5.30-5.46 (m, 1H) 6.98 (dd, J=8.10, 4.98 Hz, 1H) 7.03-7.11 (m, 1H) 8.00 (dd, J=4.88, 1.37 Hz, 1H) 8.33 (s, 1H)

### Example 19

### 1-Methylcyclopropyl (3R,4S)-4-{[6-(2,3-dihydro-1H-pyrrolo[3,2-b]pyridin-1-yl)-5-methylpyrimidin-4-yl]oxy}-3-fluoropiperidine-1-carboxylate (racemic)

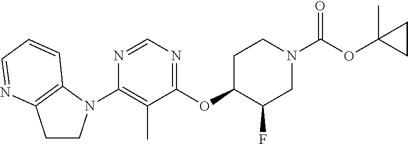

Example 19 was prepared in a manner analogous to Example 17 except using racemic (3R,4S)-tert-butyl 4-(6-(2,3-dihydro-1H-pyrrolo[3,2-b]pyridin-1-yl)-5-methylpyrimidin-4-yloxy)-3-fluoropiperidine-1-carboxylate. The crude material was dissolved in dimethyl sulfoxide (1 mL) and purified by reversed-phase HPLC (Column: Waters XBridge C₁₈19×100 mm, 5 micrometer; Mobile phase A: 0.03% ammonium hydroxide in water (v/v); Mobile phase B: 0.03% ammonium hydroxide in acetonitrile (v/v); Gradient: 90% water/10% acetonitrile linear to 0% water/100% acetonitrile in 8.5 min, hold at 0% water/100% acetonitrile to 10.0 minutes. Flow: 25 mL/min. LCMS (M+H): 428.2

### Example 20

### 1-Methylcyclopropyl 4-{[6-(5-carbamoyl-2,3-dihydro-1H-indol-1-yl)-5-methylpyrimidin-4-yl]oxy}-3-fluoropiperidine-1-carboxylate (racemic)

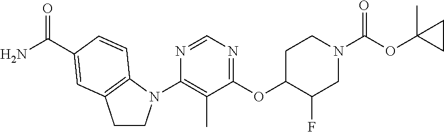

### Step A: 1-(6-(1-(tert-butoxycarbonyl)-3-fluoropiperidin-4-yloxy)-5-methylpyrimidin-4-yl)indoline-5-carboxylic acid

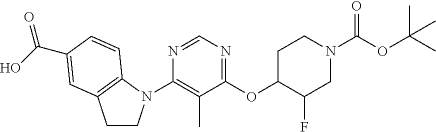

Sodium bis(trimethylsilyl)amide (0.24 mL, 1 M in tetrahydrofuran) was added drop-wise to a solution of tert-butyl 3-fluoro-4-hydroxypiperidine-1-carboxylate (48 mg, 0.22 mmol) in tetrahydrofuran (1 mL) at room temperature. The mixture was stirred for 5 minutes, before it was added drop-wise to a stirred solution of methyl 1-(6-chloro-5-methylpyrimidin-4-yl)indoline-5-carboxylate (60 mg, 0.2 mmol) in tetrahydrofuran (1 mL) at 60 degrees Celsius. The reaction mixture was stirred at 60 degrees Celsius for 2 hours. The reaction mixture was cooled to room temperature and diluted with water and ethyl acetate. The aqueous layer was extracted with ethyl acetate and the combined organic extracts were dried over magnesium sulfate, filtered and the filtrate was concentrated in vacuo. The crude 1-(6-(1-(tert-butoxycarbonyl)-3-fluoropiperidin-4-yloxy)-5-methylpyrimidin-4-yl)indoline-5-carboxylic acid was used in the next step without purification.

### Step B: tert-butyl 4-(6-(5-carbamoylindolin-1-yl)-5-methylpyrimidin-4-yloxy)-3-fluoropiperidine-1-carboxylate

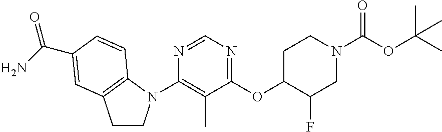

To a stirred solution of 1-(6-(1-(tert-butoxycarbonyl)-3-fluoropiperidin-4-yloxy)-5-methylpyrimidin-4-yl)indoline-5-carboxylic acid (30 mg, 0.063 mmol) in 1,4-dioxane (2 mL) was added di-teributyl carbonate (18.4 mg, 0.082 mmol) followed by pyridine (0.005 mL, 0.063 mmol). The reaction mixture was stirred at room temperature for 30 minutes before ammonium hydrogen carbonate (6.5 mg, 0.082 mmol) was added. The mixture was stirred at room temperature under nitrogen for 19 hours before it was diluted with water and ethyl acetate. The aqueous layer was extracted with ethyl acetate and the combined organic extracts were dried over magnesium sulfate, filtered and the filtrate was concentrated in vacuo. The crude tert-butyl 4-(6-(5-carbamoylindolin-1-yl)-5-methylpyrimidin-4-yloxy)-3-fluoropiperidine-1-carboxylate was used in the next step without purification.

### Step C: 1-methylcyclopropyl 4-{[6-(5-carbamoyl-2,3-dihydro-1H-indol-1-yl)-5-methylpyrimidin-4-yl]oxy}-3-fluoropiperidine-1-carboxylate (racemic)

To a stirred solution of tert-butyl 4-(6-(5-carbamoylindolin-1-yl)-5-methylpyrimidin-4-yloxy)-3-fluoropiperidine-1-carboxylate (30 mg, 0.064 mmol) in dichloromethane (0.5 mL) was added trifluoroacetic acid (0.5 mL). The mixture was stirred at room temperature for 2 hours before it was concentrated in vacuo. The residue was dissolved in tetrahydrofuran (1 mL) and triethylamine (0.05 mL) and 1-methylcyclopropyl 4-nitrophenyl carbonate (22 mg, 0.09 mmol) was added at room temperature. The reaction mixture was stirred at room temperature for 16 hours before it was diluted with water and ethyl acetate. The aqueous layer was extracted twice with ethyl acetate. The combined organic extracts were washed with saturated aqueous sodium bicarbonate, brine, dried over sodium sulfate, filtered and the filtrate was concentrated in vacuo. The residue was dissolved in dimethyl sulfoxide (1 mL) and purified by reversed-phase HPLC (Column: Waters XBridge C₁₈19×100 mm, 5 micrometer; Mobile phase A: 0.03% ammonium hydroxide in water (v/v); Mobile phase B: 0.03% ammonium hydroxide in acetonitrile (v/v); Gradient 80% water/20% acetonitrile linear to 0% water/100% acetonitrile in 10.5 minutes, hold at 0% water/100% acetonitrile to 12.0 minutes. Flow: 25 mL/min. LCMS (M+H): 470.1

### Example 21

### tert-Butyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

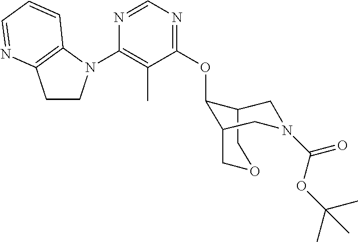

To a hot (100 degrees Celsius), stirred solution of 1-(6-chloro-5-methylpyrimidin-4-yl)-2,3-dihydro-1H-pyrrolo[3,2-b]pyridine (1.0 g, 4.05 mmol) and tert-butyl 9-anti-hydroxy-3-oxa-7-azabicyclo[3.3.1]nonane-7-carboxylate (1.08 g, 4.46 mmol) in 20 mL of 1,4-dioxane was added drop-wise a solution of sodium bis(trimethylsilyl)amide (4.86 mL, 4.86 mmol, 1.0 M in tetrahydrofuran) over 5 minutes under a nitrogen atmosphere. The mixture was stirred for 2 hours at 90 degrees Celsius before it was cooled to room temperature and diluted with water. The aqueous phase was extracted three times with ethyl acetate. The combined extracts were dried over sodium sulfate, filtered and the filtrate was concentrated in vacuo. The reside was purified by flash chromatography with 80 g silica gel column, eluting with a gradient mixture of 50% to 100% ethyl acetate to heptane to give tert-butyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate as a yellow solid (1.35 g). LCMS: 454.5 at 2.05 minutes. ¹H NMR (500 MHz, deuterochloroform) delta 1.50 (s, 9H) 1.98-2.10 (m, 2H) 2.11-2.18 (m, 3H) 3.31 (t, J=8.54 Hz, 2H) 3.38 (d, J=13.66 Hz, 1H) 3.50 (d, J=13.66 Hz, 1H) 3.88 (dd, J=14.39, 12.69 Hz, 2H) 4.08-4.26 (m, 5H) 4.33 (d, J=13.66 Hz, 1H) 5.45 (t, J=3.66 Hz, 1H) 6.96-7.05 (m, 1H) 7.05-7.12 (m, 1H) 8.04 (dd, J=4.88, 1.22 Hz, 1H) 8.38 (s, 1H).

### Example 22

### Isopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

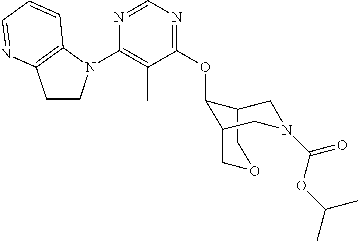

To a stirred solution of isopropyl-9-anti-hydroxy-3-oxa-7-azabicyclo[3.3.1]nonane-7-carboxylate (139 mg, 0.61 mmol) in 1 mL of tetrahydrofuran was added drop-wise sodium bis(trimethylsilyl)amide (0.81 mL, 0.81 mmol, 1.0 M in tetrahydrofuran) at room temperature. The mixture was stirred for 5 minutes before it was added to a stirred, hot (60 degrees Celsius), solution of 1-(6-chloro-5-methylpyrimidin-4-yl)-2,3-dihydro-1H-pyrrolo[3,2-b]pyridine (100 mg, 0.40 mmol) in 1 mL of tetrahydrofuran. The reaction mixture was stirred for 2 hours at 60 degrees Celsius before it was cooled to room temperature and diluted with water and ethyl acetate. The aqueous phase was extracted with ethyl acetate and the combined organic layers were dried over magnesium sulfate, filtered and the filtrate was concentrated in vacuo. The residue was purified by flash chromatography using a gradient mixture of 50% to 100% ethyl acetate to heptane to give isopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate as a white solid as (110 mg). LCMS: 440.3. ¹H NMR (500 MHz, deuterochloroform) delta 1.23-1.32 (m, 6H) 2.02-2.07 (m, 1H) 2.07-2.12 (m, 1H) 2.13 (s, 3H) 3.32 (t, J=8.42 Hz, 2H) 3.37-3.48 (m, 1H) 3.51 (d, J=13.91 Hz, 1H) 3.88 (t, J=11.59 Hz, 2H) 4.11-4.27 (m, 5H) 4.36 (d, J=13.42 Hz, 1H) 4.93-5.06 (m, 1H) 5.47 (t, J=3.66 Hz, 1H) 6.96-7.05 (m, 1H) 7.06-7.14 (m, 1H) 8.05 (dd, J=5.00, 1.34 Hz, 1H) 8.39 (s, 1H).

### Example 23

### Isopropyl (9-syn)-9-({5-methyl-6-[5-(methylsulfonyl)-2,3-dihydro-1H-indol-1-yl]pyrimidin-4-yl}oxy)-3-oxa-7-azabicyclo[3.3.1]nonane-7-carboxylate

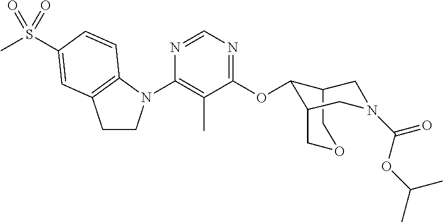

Example 23 was prepared in manner analogous to Example 1 with appropriate starting materials. The crude product was purified by silica gel chromatography, eluting with a gradient mixture of heptane and ethyl acetate from 30% to 100% ethyl acetate. This provided 200 mg of isopropyl (9-syn)-9-({5-methyl-6-[5-(methylsulfonyl)-2,3-dihydro-1H-indol-1-yl]pyrimidin-4-yl}oxy)-3-oxa-7-azabicyclo[3.3.1]nonane-7-carboxylate. ¹H NMR (400 MHz, deuterochloroform) delta 1.23-1.31 (m, 6H) 1.95-2.03 (m, 2H) 2.12-2.15 (m, 2H) 3.04 (s, 3H) 3.19-3.29 (m, 2H) 3.34 (d, J=13.66 Hz, 1H) 3.89 (d, J=14.45 Hz, 1H) 3.97 (d, J=12.30 Hz, 1H) 4.25 (br. s., 6H) 4.50 (d, J=13.86 Hz, 1H) 4.66 (d, J=12.30 Hz, 1H) 4.95-5.03 (m, 1H) 5.39-5.43 (m, 1H) 6.74 (d, J=8.39 Hz, 1 H) 7.70 (dd, J=8.49, 1.85 Hz, 1H) 7.73 (d, J=1.95 Hz, 1H) 8.44 (s, 1H).

### Example 24

### Isopropyl (9-anti)-9-({5-methyl-6-[5-(methylsulfonyl)-2,3-dihydro-1H-indol-1-yl]pyrimidin-4-yl}oxy)-3-oxa-7-azabicyclo[3.3.1]nonane-7-carboxylate

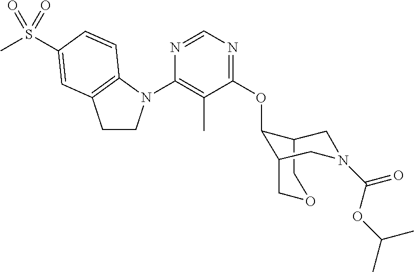

Example 24 was prepared in manner analogous to Example 1 with the appropriate starting materials. The crude product was purified by silica gel chromatography, eluting with a gradient mixture of heptane and ethyl acetate from 30% to 100% ethyl acetate. This provided 100 mg of isopropyl (9-anti)-9-({5-methyl-6-[5-(methylsulfonyl)-2,3-dihydro-1H-indol-1-yl]pyrimidin-4-yl}oxy)-3-oxa-7-azabicyclo[3.3.1]nonane-7-carboxylate. ¹H NMR (400 MHz, deuterochloroform) delta 1.23-1.31 (m, 6H) 1.95-2.03 (m, 2H) 2.12-2.15 (m, 2H) 3.04 (s, 3H) 3.19-3.29 (m, 2H) 3.34 (d, J=13.66 Hz, 1H) 3.40 (d, J=14.45 Hz, 1H) 3.50 (d, J=12.30 Hz, 1H) 3.90 (d, J=13.86 Hz, 2H) 4.18-4.38 (br. s., 6H) 4.95-5.03 (m, 1H) 5.39-5.43 (m, 1H) 6.74 (d, J=8.39 Hz, 1H) 7.70 (dd, J=8.49, 1.85 Hz, 1H) 7.73 (d, J=1.95 Hz, 1H) 8.44 (s, 1 H).

### Example 25

### 1-Methylcyclopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

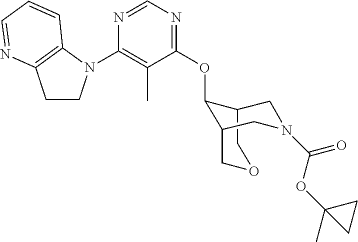

### Step A: (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane

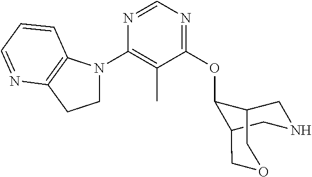

To stirred solution of tert-butyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate (1.30 g, 2.87 mmol) in 6 mL of dichloromethane was added 3 mL of trifluoroacetic acid (TFA) at room temperature. The resulting solution was stirred at room temperature for 2 hours and then concentrated in vacuo. The residue was used without purification in the next step.

### Step B: 1-Methylcyclopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

To a stirred solution of (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane (60 mg, 0.10 mmol) in 1 mL of tetrahydrofuran was added triethylamine (0.06 mL, 0.41 mmol) followed by 1-methylcyclopropyl 4-nitrophenyl carbonate (49 mg, 0.21 mmol) at room temperature. The reaction mixture was stirred under a nitrogen atmosphere for 16 hours before it was diluted with water and extracted with ethyl acetate. The combined organic layers were washed twice with saturated aqueous sodium bicarbonate, brine, dried over sodium sulfate, filtered, and the filtrate was concentrated in vacuo. The crude residue was purified by flash chromatography (4 g silica: 50% to 100% heptane to ethyl acetate) to afford 1-methylcyclopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate as a white solid (20 mg). LCMS m/z: 452.3. ¹H NMR (500 MHz, deuterochloroform) delta 0.58-0.70 (m, 2H) 0.84-1.01 (m, 2H) 1.59 (s, 3H) 2.01 (br. s., 1H) 2.04-2.10 (m, 1H) 2.10-2.17 (m, 3H) 3.31 (t, J=8.42 Hz, 2H) 3.37-3.54 (m, 2H) 3.86 (t, J=11.34 Hz, 2 H) 4.06-4.26 (m, 5H) 4.30-4.44 (m, 1H) 4.36 (d, J=13.66 Hz, 1H) 5.45 (t, J=3.66 Hz, 1H) 6.95-7.05 (m, 1H) 7.05-7.13 (m, 1H) 8.04 (dd, J=4.88, 0.98 Hz, 1H) 8.38 (s, 1 H).

### Example 26

### 1-Ethylcyclopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

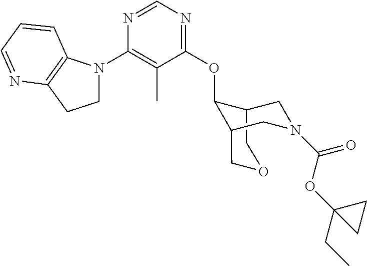

Example 26 was prepared in manner analogous to Example 25 using 1-ethylcyclopropyl 4-nitrophenyl carbonate. The crude material was dissolved in dimethyl sulfoxide (1 mL) and purified by reversed-phase HPLC (Column: Waters Sunfire C₁₈ 19×100 mm, 5 micrometer; Mobile phase A: 0.05% trifluoroacetic acid in water (v/v); Mobile phase B: 0.05% trifluoroacetic acid in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/min. Purification in this way proved 23 mg of 1-ethylcyclopropyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate. LCMS (M+H): 466.3

### Example 27

### 1-Methylcyclobutyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

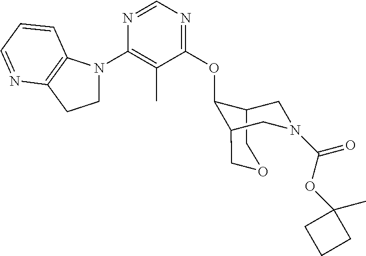

Example 27 was prepared in manner analogous to Example 25 using the 1-methylcyclobutyl 4-nitrophenyl carbonate. The crude residue was dissolved in dimethyl sulfoxide (1 mL) and purified by reversed-phase HPLC (Column: Waters Sunfire C₁₈ 19×100 mm, 5 micrometer; Mobile phase A: 0.05% trifluoroacetic acid in water (v/v); Mobile phase B: 0.05% trifluoroacetic acid in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/min. Purification in this way provided 18 mg of 1-methylcyclobutyl (9-anti)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate. LCMS (M+H): 466.3

### Example 28

### 1-Methylcyclopropyl (9-syn)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

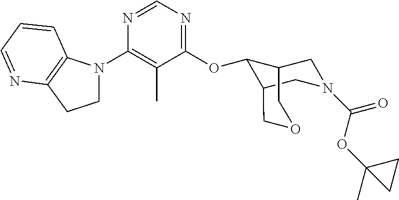

Example 28 was prepared in manner analogous to Example 25 using tert-butyl 9-syn-hydroxy-3-oxa-7-azabicyclo[3.3.1]nonane-7-carboxylate.

¹H NMR (400 MHz, deuterochloroform) delta 0.55-0.65 (m, 2H) 0.82-0.97 (m, 2H) 1.55 (s, 3H) 1.87-2.01 (m, 2H) 2.11 (s, 3H) 3.18 (d, J=13.68 Hz, 1H) 3.22-3.34 (m, 3H) 3.79 (d, J=11.53 Hz, 1H) 3.93 (d, J=11.33 Hz, 1H) 4.02-4.21 (m, 4H) 4.35 (d, J=13.88 Hz, 1H) 4.61 (d, J=13.68 Hz, 1H) 5.34 (t, J=3.32 Hz, 1H) 6.92-7.02 (m, 1H) 7.02-7.10 (m, 1H) 8.00 (dd, J=4.98, 1.47 Hz, 1H) 8.34 (s, 1H).

### Example 29

### Isopropyl (9-syn)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate

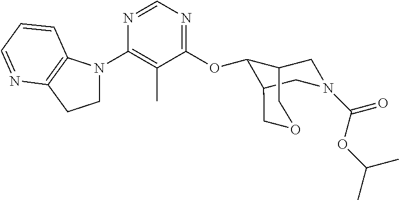

Example 29 was prepared in manner analogous to Example 28 using isopropyl-9-syn-hydroxy-3-oxa-7-azabicyclo[3.3.1]nonane-7-carboxylate. The crude residue was dissolved in dimethyl sulfoxide (1 mL) and purified by reversed-phase HPLC (Column: Waters Sunfire C₁₈19×100 mm, 5 micrometer); Mobile phase A: 0.05% trifluoroacetic acid in water (v/v); Mobile phase B: 0.05% trifluoroacetic acid in acetonitrile (v/v); Gradient 80% water/20% acetonitrile linear to 40% water/60% acetonitrile over 10.0 minutes to 0% water/100% acetonitrile in 10.5 minutes, hold at 0% water/100% acetonitrile to 12.0 minutes; Flow: 25 mL/minute. Purification in this way provided 14 mg of isopropyl (9-syn)-9-{[6-(2,3-dihydro-1H-pyrrolo[3,2-b]pyridin-1-yl)-5-methylpyrimidin-4-yl]oxy}-3-oxa-7-azabicyclo[3.3.1]nonane-7-carboxylate as the trifluoroacetic acid salt. LCMS (M+H): 440.3.

### Example 30

### tert-Butyl (3S,4R)-4-{[6-(6,7-dihydro-5H-pyrrolo[3,2-c]pyridazin-5-yl)-5-methylpyrimidin-4-yl]oxy}-3-fluoropiperidine-1-carboxylate

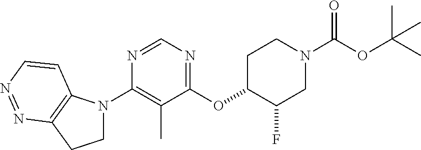

To a stirred solution of (3S,4R)-tert-butyl 3-fluoro-4-hydroxypiperidine-1-carboxylate (34 mg, 0.16 mmol) in tetrahydrofuran (0.5 mL) was added sodium tert-butoxide (0.17 mL, 0.17 mmol, 1 M in tetrahydrofuran) at room temperature. After 20 minutes this mixture was added to a stirred, cold (0 degrees Celsius), suspension of 5-(6-chloro-5-methylpyrimidin-4-yl)-6,7-dihydro-5H-pyrrolo[3,2-c]pyridazine (Preparation 32) (35 mg, 0.14 mmol) in tetrahydrofuran (0.5 mL). The resulting solution was stirred at 0 degrees Celsius for 80 minutes. The cold bath was removed and the reaction was allowed to warm to room temperature. After 5.5 hours at room temperature the reaction mixture was diluted with water and brine and extracted with ethyl acetate (3×15 mL). The combined organic extracts were washed with brine diluted with one volume of water, dried (sodium sulfate), filtered, and the filtrate was concentrated in vacuo to 60 mg of a light yellow residue. This material was purified by chromatography using 40 g of basic alumina, eluting with 20% methanol in dichloromethane. The resulting material dissolved in dimethyl sulfoxide (1 mL) and purified by reversed-phase HPLC (Column: Waters XBridge C18 19×100 mm, 5 micrometer); Mobile phase A: 0.03% ammonium hydroxide in water (v/v); Mobile phase B: 0.03% ammonium hydroxide in acetonitrile (v/v); Gradient: 85% water/15% acetonitrile linear to 0% water/100% acetonitrile in 8.5 min, hold at 0% water/100% acetonitrile to 10.0 minutes. Flow: 25 mL/min. LCMS (M+H): 431.28.

### Example 31

### 1-Methylcyclopropyl (3S,4R)-4-{[6-(6,7-dihydro-5H-pyrrolo[3,2-c]pyridazin-5-yl)-5-methylpyrimidin-4-yl]oxy}-3-fluoropiperidine-1-carboxylate

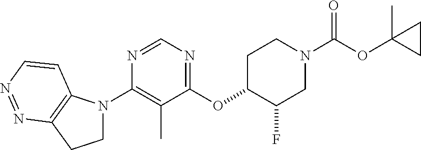

Example 31 was prepared in manner analogous to Example 17 starting with tert-butyl (3S,4R)-4-{[6-(6,7-dihydro-5H-pyrrolo[3,2-c]pyridazin-5-yl)-5-methylpyrimidin-4-yl]oxy}-3-fluoropiperidine-1-carboxylate. MS (M+H): 429.2. ¹H NMR (400 MHz, deuterochloroform) delta 0.66 (br. s., 2H) 0.91 (br. s., 2H) 1.58 (s, 3H) 1.93 (br. s., 1 H) 2.09 (s, 3H) 2.17 (d, J=11.53 Hz, 1H) 3.19 (br. s., 1H) 3.41 (br. s., 1H) 3.52 (1, J=8.40 Hz, 2H) 3.79-4.12 (m, 1H) 4.14-4.35 (m, 3H) 4.75-5.05 (m, 1H) 5.44 (d, J=14.07 Hz, 1H) 6.59 (d, J=5.86 Hz, 1H) 8.44 (s, 1H) 8.69 (d, J=5.67 Hz, 1H).

### Example 32

### 1-Methylcyclopropyl (3R,4S)-4-{[6-(5-carbamoyl-2,3-dihydro-1H-pyrrolo[3,2-b]pyridin-1-yl)pyrimidin-4-yl]oxy}-3-fluoropiperidine-1-carboxylate

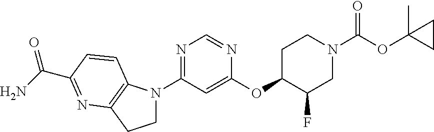

### Step A: Methyl 1-(6-((3R,4S)-3-fluoro-1-((1-methylcyclopropoxy)carbonyl)piperidin-4-yloxy)pyrimidin-4-yl)-2,3-dihydro-1H-pyrrolo[3,2-b]pyridine-5-carboxylate (racemic)

To a solution of methyl 2,3-dihydro-1H-pyrrolo[3,2-b]pyridine-5-carboxylate (35 mg, 0.20 mmol) and (3R,4S)-1-methylcyclopropyl 4-(6-chloropyrimidin-4-yloxy)-3-fluoropiperidine-1-carboxylate (racemic) (64.6 mg, 0.020 mmol) in tert-butanol (1 mL) and toluene (1 mL) was added cesium carbonate (163 mg). The mixture was degassed with a stream of nitrogen gas. Bis(triphenylphosphine)palladium(II) dichloride (14 mg) was added and the mixture was again degassed with nitrogen for several minutes. The resulting mixture was heated at reflux (115 degrees Celsius) for 18 hours. The mixture was cooled to room temperature, diluted with ethyl acetate and the mixture was filtered through diatomaceous earth. The filtrate was washed with water, dried over magnesium sulfate, filtered, and the filtrate was concentrated in vacuo. The residue was purified by silica gel chromatography, eluting with a gradient mixture of 50% to 90% ethyl acetate to heptane to give methyl 1-(6-((3R,4S)-3-fluoro-1-((1-methylcyclopropoxy)carbonyl)piperidin-4-yloxy)pyrimidin-4-yl)-2,3-dihydro-1H-pyrrolo[3,2-b]pyridine-5-carboxylate (racemic) as a pale yellow solid (80 mg). LCMS (M+H): 472.0.

### Step B: 1-(6-((3R,4S)-3-Fluoro-1-((1-methylcyclopropoxy)carbonyl)piperidin-4-yloxy)pyrimidin-4-yl)-2,3-dihydro-1H-pyrrolo[3,2-b]pyridine-5-carboxylic acid (racemic)

To a stirred solution of methyl 1-(6-((3R,4S)-3-fluoro-1-((1-methylcyclopropoxy)carbonyl)piperidin-4-yloxy)pyrimidin-4-yl)-2,3-dihydro-1H-pyrrolo[3,2-b]pyridine-5-carboxylate (racemic) (50 mg, 0.11 mmol) in a 3:1 solution of tetrahydrofuran and water (2 mL) was added lithium hydroxide monohydrate (10 mg, 0.22 mmol). The reaction mixture was stirred at room temperature for 18 hours before 1N aqueous hydrochloric acid was added until the solution was approxitemately pH 2. The precipitate was collected by filtration to provide 40 mg of 1-(6-((3R,4S)-3-fluoro-1-((1-methylcyclopropoxy)carbonyl)piperidin-4-yloxy)pyrimidin-4-yl)-2,3-dihydro-1H-pyrrolo[3,2-b]pyridine-5-carboxylic acid (racemic) as a white solid. This material was used in the subsequent step without purification.

### Step C: 1-Methylcyclopropyl (3R,4S)-4-{[6-(5-carbamoyl-2,3-dihydro-1H-pyrrolo[3,2-b]pyridin-1-yl)pyrimidin-4-yl]oxy}-3-fluoropiperidine-1-carboxylate (racemic)

To a stirred solution of 1-(6-((3R,4S)-3-fluoro-1-((1-methylcyclopropoxy)carbonyl)piperidin-4-yloxy)pyrimidin-4-yl)-2,3-dihydro-1H-pyrrolo[3,2-b]pyridine-5-carboxylic acid (racemic) (25 mg, 0.055 mmol) in 1,4-dioxane (2 mL) was added di-tert-butyl carbonate (25 mg, 0.11 mmol) and pyridine (8.9 microliters, 0.11 mmol). The mixture was stirred at room temperature for 30 minutes before ammonium hydrogen carbonate (8.7 mg, 0.11 mmol) was added. The mixture was stirred at room temperature under a nitrogen atmosphere for 19 hours. The solids from this reaction mixture were collected by filtration, rinsing with was to give 1-methylcyclopropyl (3R,4S)-4-{[6-(5-carbamoyl-2,3-dihydro-1H-pyrrolo[3,2-b]pyridin-1-yl)pyrimidin-4-yl]oxy}-3-fluoropiperidine-1-carboxylate (racemic) as a white solid (20 mg) after drying under vacuum. LCMS (M+H): 457.1. ¹H NMR (methanol-d4) delta 8.71 (d, J=8.4 Hz, 1H), 8.46 (d, J=0.8 Hz, 1H), 7.90 (d, J=8.6 Hz, 1H), 6.23 (d, J=0.8 Hz, 1H), 5.29-5.45 (m, 1H), 4.84-5.03 (m, 1H), 4.22 (br. S., 1H), 4.07-4.14 (m, 2H), 3.94 (br. S., 1H), 3.36 (t, J=8.6 Hz, 2H), 3.11 (br. S., 2H), 1.87-2.02 (m, 2H), 1.51 (s, 3H), 0.81-0.91 (m, 2H), 0.58-0.68 (m, 2H).

### Example 33

### 1-Methylcyclopropyl 4-({6-[5-(dimethylcarbamoyl)-2,3-dihydro-1H-pyrrolo[3,2-b]pyridin-1-yl]pyrimidin-4-yl}oxy)piperidine-1-carboxylate

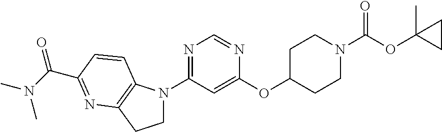

Example 33 was prepared from N,N-dimethyl-2,3-dihydro-1H-pyrrolo[3,2-b]pyridine-5-carboxamide and 1-methylcyclopropyl 4-(6-chloropyrimidin-4-yloxy)piperidine-1-carboxylate in a manner analogous to Example 32, Step A. The crude material was dissolved in dimethyl sulfoxide (1 mL) and purified by reversed-phase HPLC (Column: Waters XBridge C18 19×100 mm, 5 micrometer); Mobile phase A: 0.05% trifluoroacetic acid in water (v/v); Mobile phase B: 0.05% trifluoroacetic acid in acetonitrile (v/v); Gradient: 85% water/15% acetonitrile linear to 0% water/100% acetonitrile in 8.5 minutes, hold at 0% water/100% acetonitrile to 10.0 minutes. Flow: 25 mL/min. LCMS (M+H): 467.3.

Throughout this application, various publications are referenced. The disclosures of these publications in their entireties are hereby incorporated by reference into this application for all purposes.

It will be apparent to those skilled in the art that various modifications and variations can be made in the invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A compound of the formula (I): 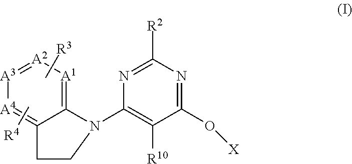
in which
X is 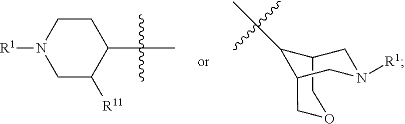
R¹ is —C(O)—O—R⁵ or 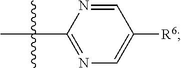
R² is hydrogen, cyano, or methyl;
R³ is hydrogen, OH, halogen, cyano, CF₃, OCF₃, C₁-C₅ alkoxy, or C₁-C₅ alkyl;
R⁴ is SO₂—R⁷ or —NH—(CH₂)₂—OH;
R⁵ is C₁-C₅ alkyl, C₃-C₆ cycloalkyl, or C₃-C₆ cycloalkyl in which one carbon atom of said cycloalkyl moiety is optionally substituted with methyl or ethyl;
R⁶ is CF₃, C₁-C₅ alkyl, halogen, cyano, or C₃-C₆ cycloalkyl;
R⁷ is C₃-C₆ cycloalkyl, C₁-C₅ alkyl, NH₂, or —(CH₂)₂—OH;
R⁸ is hydrogen or C₁-C₅ alkyl,
R⁹ is hydrogen, C₁-C₅ alkyl, C₃-C₆ cycloalkyl, —CH₂—CH₂—OH, —CH₂—CH₂—O—CH₃, —CH₂—CH₂—CH₂—O—CH₃, —CH₂—CH₂—CH₂—OH, 3-oxetanyl, or 3-hydroxycyclobutyl,
R¹⁰ is hydrogen, cyano, nitro, CF₃, OCF₃, C₃-C₆ cycloalkyl, C₁-C₅ alkoxy, or C₁-C₅ alkyl;
R¹¹ is hydrogen, C₁-C₅ alkyl, or halogen; and
A¹, A², A³, and A⁴, are each independently CH, N-oxide, or N;
with the proviso that:
a) no more than 2 of A¹, A², A³, and A⁴ are N;
b) no more than 1 of A¹, A², A³, and A⁴ are N-oxide; and
c) when A¹-A⁴ forms a phenyl ring, X is 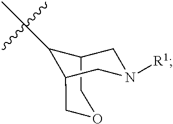
or a pharmaceutically acceptable salt thereof.

2. A compound according to claim 1 in which X is

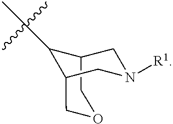

3. A compound according to claim 1 in which X is

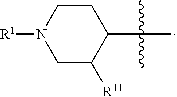

4. A compound according to claim 1 in which A¹-A⁴ forms a ring in which one or two of A¹, A², A³, and A⁴ are N.

5. A compound according to claim 1 in which A¹-A⁴ forms a pyridyl ring.

6. A compound according to claim 1 in which R⁴ is —SO₂R⁷.

7. A compound according to claim 1 in which R¹ is —C(O)—O—R⁵.

8. A compound according to claim 1 in which R³ is fluoro or hydrogen.

9. A compound according to claim 1 in which R² is hydrogen or cyano.

10. A compound selected from the group consisting of:
Isopropyl 9-anti-({6-[5-(methylsulfonyl)-2,3-dihydro-1H-indol-1-yl]pyrimidin-4-yl}oxy)-3-oxa-7-azabicyclo[3.3.1]nonane-7-carboxylate; and
Isopropyl 9-syn-({6-[5-(methylsulfonyl)-2,3-dihydro-1H-indol-1-yl]pyrimidin-4-yl}oxy)-3-oxa-7-azabicyclo[3.3.1]nonane-7-carboxylate;
or a pharmaceutically acceptable salt thereof.

11. A pharmaceutical composition comprising a compound according to claim 10 present in a therapeutically effective amount, in admixture with at least one pharmaceutically acceptable excipient.

12. The composition of claim 11 further comprising at least one additional pharmaceutical agent selected from the group consisting of an anti-obesity agent and an anti-diabetic agent.

13. The composition of claim 12 wherein said anti-obesity agent is selected from the group consisting of dirlotapide, mitratapide, implitapide, R56918 (CAS No. 403987), CAS No. 913541-47-6, lorcaserin, cetilistat, PYY₃₋₃₆, naltrexone, oleoyl-estrone, obinepitide, pramlintide, tesofensine, leptin, liraglutide, bromocriptine, orlistat, exenatide, AOD-9604 (CAS No. 221231-10-3) and sibutramine.

14. The composition of claim 12 wherein said anti-diabetic agent is selected from the group consisting of metformin, acetohexamide, chlorpropamide, diabinese, glibenclamide, glipizide, glyburide, glimepiride, gliclazide, glipentide, gliquidone, glisolamide, tolazamide, tolbutamide, tendamistat, trestatin, acarbose, adiposine, camiglibose, emiglitate, miglitol, voglibose, pradimicin-Q, salbostatin, balaglitazone, ciglitazone, darglitazone, englitazone, isaglitazone, pioglitazone, rosiglitazone, troglitazone, exendin-3, exendin-4, trodusquemine, reservatrol, hyrtiosal extract, sitagliptin, vildagliptin, alogliptin and saxagliptin.

15. A method for the treatment of diabetes comprising the administration of an effective amount of compound according to claim 10 to a patient in need thereof.

16. A method for treating a metabolic or metabolic-related disease, condition or disorder comprising the step of administering to a patient a therapeutically effective amount of a compound of claim 10.

17. A method for treating a condition selected from the group consisting of hyperlipidemia, type I diabetes, type II diabetes mellitus, idiopathic type I diabetes (Type Ib), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance, hyper apo B lipoproteinemia, Alzheimer's, schizophrenia, impaired cognition, inflammatory bowel disease, ulcerative colitis, Crohn's disease, and irritable bowel syndrome, comprising the administration of an effective amount of a compound according to claim 10.

18. A method for treating a metabolic or metabolic-related disease, condition or disorder comprising the step of administering to a patient in need of such treatment two separate pharmaceutical compositions comprising
(i) a first composition according to claim 13; and
(ii) a second composition comprising at least one additional pharmaceutical agent selected from the group consisting of an anti-obesity agent and an anti-diabetic agent, and at least one pharmaceutically acceptable excipient.

19. The method of claim 18 wherein said first composition and said second composition are administered simultaneously.

20. The method of claim 18 wherein said first composition and said second composition are administered sequentially and in any order.

21. (canceled)

22. (canceled)
